(19) United States
(12) Patent Application Publication
(10) Pub. No.: US 2025/0082651 A1
(43) Pub. Date: Mar. 13, 2025

(54) GANAXOLONE FOR USE IN TREATING GENETIC EPILEPTIC DISORDERS

(71) Applicant: Marinus Pharmaceuticals, Inc., Radnor, PA (US)

(72) Inventors: Lorianne Masuoka, Chestnut Hill, MA (US); Jaakko Lappalainen, Wilmington, DE (US)

(21) Appl. No.: 18/612,277

(22) Filed: Mar. 21, 2024

Related U.S. Application Data
(63) Continuation of application No. 18/365,280, filed on Aug. 4, 2023, now abandoned.
Application No. 18/365,280 is a continuation of application No. 17/703,331, filed on Mar. 24, 2022, now abandoned.
Application No. 17/703,331 is a continuation of application No. 17/681,001, filed on Feb. 25, 2022, now abandoned.
Application No. 17/681,001 is a continuation of application No. 17/368,602, filed on Jul. 6, 2021, now abandoned.
Application No. 17/368,602 is a continuation of application No. 17/065,174, filed on Oct. 7, 2020, now abandoned.
Application No. 17/065,174 is a continuation of application No. 16/185,677, filed on Nov. 9, 2018, now abandoned.
(60) Provisional application No. 62/584,403, filed on Nov. 10, 2017.

(51) Int. Cl. A61K 31/565; A61K 9/00; A61K 9/08; A61K 9/48; A61K 47/12; A61K 47/24; A61K 47/26; A61K 47/34; A61K 47/38; A61P 25/08

(52) U.S. Cl. CPC A61K 31/565; A61K 9/0019; A61K 9/0095; A61K 9/08; A61K 9/4858; A61K 9/4866; A61K 47/12; A61K 47/24; A61K 47/26; A61K 47/34; A61P 25/08; A61K 9/485; A61K 47/38

(57) ABSTRACT

The disclosure provides a method of treating a mammal having a genetic epileptic disorder, comprising chronically administering a pharmaceutically acceptable pregnenolone neurosteroid to a mammal having a genetic epileptic disorder in an amount effective to reduce the seizure frequency in the mammal. In certain preferred embodiments, the mammal is a human patient who has a CDKL5 genetic mutation. In certain preferred embodiments, the patient has a low endogenous level of a neurosteroid(s). In certain preferred embodiments, the pregnenolone neurosteroid is ganaxolone.

### CROSS-REFERENCE TO RELATED APPLICATION

This application claims priority from U.S. Provisional Application No. 62/584,403, filed on Nov. 10, 2017, the disclosure of which is hereby incorporated by reference in its entirety for all purposes.

### BACKGROUND OF THE INVENTION

Infantile epileptic encephalopathies and rare pediatric epilepsies are conditions of significant unmet medical need. These conditions include PCDH19-related epilepsy, CDKL5 Deficiency Disorder (CDD), Dravet Syndrome, Lennox-Gastaut syndrome (LGS), Continuous Sleep Wave in Sleep (CSWS), Epileptic Status Epilepticus in Sleep (ESES), and other intractable and refractory genetic epilepsy conditions that clinically resemble PCDH19-related epilepsy, CDKL5 Deficiency Disorder, Dravet Syndrome, LGS, CSWS, and ESES.

PCDH19-related epilepsy is a serious and rare epileptic syndrome that predominantly affects females. The condition is caused by an inherited mutation of the protocadherin 19 (PCDH19) gene, located on the X chromosome, and is characterized by early-onset and highly variable cluster seizures, cognitive and sensory impairment, and behavioral disturbances. Currently, there are no approved therapies for PCDH19-related epilepsy, nor is there any effective standard of care therapy.

CDKL5 is a rare X linked genetic disorder that results in early onset, difficult to control seizures, and severe neuro-developmental impairment. The most common feature of CDKL5 deficiency disorder is early drug-resistant epilepsy, usually starting in the first months of life. Seizures are generally highly polymorphic. Complex partial seizures, infantile spasms, myoclonic, generalized tonic-clonic, and tonic seizures have all been reported. Many different seizure types can also occur in the same patient, changing with time very often. Patients treated with antiepileptic drugs (“AEDs”) experience a brief seizure-free honeymoon period, which, unfortunately, is followed by relapses (Kilstrup-Nielsen et al, 2012). CDKL5 deficiency disorder is among the epileptic encephalopathies that are most refractory to treatment.

The lack of meaningful treatment benefit from AEDs or any other intervention in CDKL5 is well summarized by the patient advocacy group, CDKL5UK: “At the moment, we are not aware of any particular medication that is beneficial for people with CDKL5 Deficiency Disorder. Some have implanted vagus nerve stimulators; this was beneficial for some people. Some people find that their child will not respond to any anti-epileptic medication and their consultant makes the difficult decision to decide to stop all anti-epileptic medication. Many parents have noticed that their child's seizures are much better when they are fasting, though the ketogenic diet has not worked for most people. We hope that an improved understanding of the CDKL5 gene and its function will lead on to new and more effective treatments.”

No therapeutic agent has been found to be uniformly effective in the treatment of epileptic encephalopathies and rare pediatric epilepsies, and often multiple therapeutic agents (e.g., anticonvulsants) are used together to treat PCDH19-related epilepsy, Dravet Syndrome, Lennox-Gastaut syndrome (LGS), Continuous Sleep Wave in Sleep (CSWS), Epileptic Status Epilepticus in Sleep (ESES), and other intractable epilepsy conditions and refractory genetic epilepsy conditions that clinically resemble PCDH19-related epilepsy, CDKL5 Deficiency Disorder, Dravet Syndrome, LGS, CSWS, and ESES.

There are also no approved or licensed therapies in the United States for the treatment of patients with CDKL5 deficiency disorder. There is also no accepted standard of care, nor are there guidelines from authoritative scientific bodies regarding the treatment of these patients. However, most antiepileptic drugs (“AEDs”), including steroids/adrenocorticotropic hormone (ACTH), ketogenic diet, vagal nerve stimulation, and corpus callosotomy (to disrupt inter-hemispheric connections for reduction of secondarily generalized seizures) have been tried to treat this condition.

Efficacy of multiple AEDs and ketogenic diet in patients with the CDKL5 mutation is very low. Newer drugs tend to be less sedating, have fewer adverse effects on memory and learning, and are less likely to cause allergic reactions and serious side effects. However, some of the most commonly used AEDs to treat CDKL5 deficiency disorder-associated seizures are associated with the following severe adverse effects:
Cognitive side effects are a considerable concern with topiramate.
Felbamate can cause aplastic anemia or liver failure.
Vigabatrin can permanently reduce a child's field of vision.
Stevens-Johnson syndrome, a severe allergic drug reaction, remains a concern with lamotrigine.

Compared to the pre-1990 drugs, many of the newer drugs have a broader range of action, making them more likely to work for generalized seizures. However, some, like gabapentin, pregabalin, oxcarbazepine, and tiagabine, seem to work only for seizures that have a focal onset.

Seizures in PCDH19-related epilepsy, CDKL5 Deficiency Disorder, Dravet Syndrome, Lennox-Gastaut syndrome (LGS), Continuous Sleep Wave in Sleep (CSWS), Epileptic Status Epilepticus in Sleep (ESES), and other intractable and refractory genetic epilepsy conditions that share common seizure types and clinically resemble PCDH19-related epilepsy, CDKL5 Deficiency Disorder, Dravet Syndrome, LGS, CSWS, and ESES at times become treatment resistant to conventional antiepileptic and anticonvulsant agents.

More effective therapies, particularly those with minimal side effects compared to existing therapies, are needed for these children with refractory epileptic encephalopathies and rare pediatric epilepsies.

The present invention fulfills this need by providing oral liquid neurosteroid formulations, oral solid neurosteroid formulations and injectable neurosteroid formulations for treatment of PCDH19-related epilepsy, CDKL5 Deficiency Disorder, Dravet Syndrome, LGS, CSWS, and ESES, and like conditions; and methods of diagnosis and treatment of PCDH19-related epilepsy, CDKL5 Deficiency Disorder, Dravet Syndrome, LGS, CSWS, and ESES, and like conditions

### OBJECTS AND SUMMARY OF THE INVENTION

It is an object of the invention to provide a treatment for early infantile epileptic encephalopathy.

It is another object of the invention to utilize ganaxolone's gamma-aminobutyric acid (GABA)-ergic mechanism of action to provide a therapeutic benefit for seizures, neuropsychological disorders, and sleep disturbances associated with PCDH19-related epilepsy, CDKL5 epileptic encephalopathy, Dravet Syndrome, Lennox-Gastaut syndrome (LGS), Continuous Sleep Wave in Sleep (CSWS), Epileptic Status Epilepticus in Sleep (ESES), and other intractable and refractory genetic epilepsy conditions that share common seizure types and clinically resemble PCDH19-related epilepsy, CDKL5 Deficiency Disorder, Dravet Syndrome, LGS, CSWS, and ESES.

In furtherance of the above objects and others, the present invention is directed in part to oral immediate release formulations comprising particles comprising (i) a pregnenolone neurosteroid (e.g., ganaxolone) and (ii) one or more pharmaceutically acceptable excipient(s) (e.g., oral suspensions, tablets or capsules), wherein the particles have a particle size that ensures an absence of agglomeration following dispersal in simulated gastrointestinal fluids (SGF and/or SIF) and does not change upon storage of the formulation at 25° C./60% RH for 1 month. In the preferred embodiments, the formulation releases not less than about 70% or about 80% of the pregnelone neurosteroid at 45 minutes of placing the formulation into 500 ml of a dissolution medium (e.g., 5% SLS in SGF (Simulated Gastric Fluid) and/or 5% SLS in SIF (Simulated Intestinal Fluid)) at 37° C.±0.5° C. in USP Apparatus 1 (Basket) at 100 rpm, and, after a single dose and/or multiple dose administrations, provides a plasma level of the pregnenolone neurosteroid of from about 55 ng/mL, about 60 ng/ml or about 65 ng/ml to a plasma level of the pregnenolone neurosteroid of from about 240 ng/ml to 400 ng/ml (e.g., 262 ng/mL) for a time period of at least about 6 hours, about 7 hours, about 8 hours, about 9 hours, about 10 hours, or about 12 hours. In some of these embodiments, the volume weighted median diameter of the particles is from about 250 nm to about 450 nm (e.g., about 332 nm). In some of the embodiments, the particles have a D(10) particle size of from about 200 nm to about 220 nm, a D(50) particle size of from about 250 nm to about 450 nm and a D(90) particle size of from about 480 nm to about 700 nm, and the formulation is free from cyclodextrins, including sulfoalkyl ether cyclodextrins and modified forms thereof, and is for treating a disorder selected from the group comprising or consisting of from PCDH19-related epilepsy, CDKL5 epileptic encephalopathy, Dravet Syndrome, Lennox-Gastaut syndrome (LGS), Continuous Sleep Wave in Sleep (CSWS), Epileptic Status Epilepticus in Sleep (ESES), and other intractable and refractory genetic epilepsy conditions that clinically resemble PCDH19-related epilepsy, CDKL5 Deficiency Disorder, Dravet Syndrome, LGS, CSWS, and ESES, in a human.

The present invention is also directed in part to an oral immediate release formulation comprising particles comprising (i) ganaxolone and (ii) one or more pharmaceutically acceptable excipient(s) (e.g., oral suspensions, tablets or capsules), wherein the particles have a mean particle size of about 0.3 micron (i.e., volume weighted median diameter (D50) of about 0.3 micron); the particle size does not change upon storage of the formulation at 25° C./60% RH for 1 month; the formulation releases not less than about 70% or about 80% of ganaxolone at 45 minutes of placing the formulation into 500 ml of a dissolution medium (e.g., 5% SLS in SGF (Simulated Gastric Fluid) and/or 5% SLS in SIF (Simulated Intestinal Fluid)) at 37° C.±0.5° C. in USP Apparatus 1 (Basket) at 100 rpm; the formulation provides, after a single dose and/or multiple doses, a plasma level of ganaxolone of from about 55 ng/mL, about 60 ng/ml or about 65 ng/ml to a plasma level of from about 240 ng/ml to 400 ng/ml (e.g., 262 ng/mL) for at least 6 hours to 12 hours after administration, and is for treatment of a disorder selected from the group comprising or consisting from PCDH19-related epilepsy, CDKL5 epileptic encephalopathy, Dravet Syndrome, Lennox-Gastaut syndrome (LGS), Continuous Sleep Wave in Sleep (CSWS), Epileptic Status Epilepticus in Sleep (ESES), and other intractable and refractory genetic epilepsy conditions that clinically resemble PCDH19-related epilepsy, CDKL5 Deficiency Disorder, Dravet Syndrome, LGS, CSWS, and ESES, in a human. The plasma level of ganaxolone of from about 55 ng/mL, about 60 ng/ml or about 65 ng/ml to a plasma level of from about 240 ng/ml to 400 ng/ml (e.g., 262 ng/mL) may be provided after a fasting and/or fed administration of the formulation. In some of these embodiments, the mean particle size of about 0.3 micron is critical for providing the dissolution of not less than about 70% or about 80% of the pregnelone neurosteroid at 45 minutes of placing the formulation into a simulated gastrointestinal fluid (SGF and/or SIF) and the plasma level of the pregnenolone neurosteroid of from about 55 ng/mL, about 60 ng/ml or about 65 ng/ml to a plasma level of the pregnenolone neurosteroid of from about 240 ng/ml to 400 ng/ml (e.g., 262 ng/mL) for the time period of at least about 6 hours, about 7 hours, about 8 hours, about 9 hours, about 10 hours, or about 12 hours.

The present invention is also directed in part to an immediate release formulations comprising particles comprising (i) ganaxolone and (ii) one or more pharmaceutically acceptable excipient(s) (e.g., oral suspensions, tablets or capsules), wherein the particles have a mean particle size of about 0.3 micron; the particle size does not change upon storage of the formulation at 25° C./60% RH for 2 months and/or 3 months and/or 4 months; the formulation releases not less than 80% of ganaxolone at 45 minutes of placing the formulation into 500 ml of a dissolution medium (e.g., 5% SLS in SGF (Simulated Gastric Fluid) and/or 5% SLS in SIF (Simulated Intestinal Fluid)) at 37° C.±0.5° C. in USP Apparatus 1 (Basket) at 100 rpm); the formulation provides a plasma level of ganaxolone of from about 55 ng/mL, about 60 ng/ml or about 65 ng/ml to a plasma level of from about 240 ng/ml to 400 ng/ml (e.g., 262 ng/mL) for at least 6 hours to 12 hours after administration is for treatment of a disorder selected from the group comprising or consisting from PCDH19-related epilepsy, CDKL5 epileptic encephalopathy, Dravet Syndrome, Lennox-Gastaut syndrome (LGS), Continuous Sleep Wave in Sleep (CSWS), Epileptic Status Epilepticus in Sleep (ESES), and other intractable and refractory genetic epilepsy conditions that clinically resemble PCDH19-related epilepsy, CDKL5 Deficiency Disorder, Dravet Syndrome, LGS, CSWS, and ESES, in a human.

The invention is further directed to a method of treating a mammal having a genetic epileptic disorder, comprising chronically administering a pharmaceutically acceptable pregnenolone neurosteroid (e.g., ganaxolone) to a mammal having a genetic epileptic disorder in an amount effective to reduce the seizure frequency in the mammal. In certain preferred embodiments, the mammal is human; and the epilepsy disorder is a genetic epileptic disorder, e.g., an early infantile epileptic encephalopathy. In certain preferred embodiments, the disorder is selected from, e.g., cyclin-dependent kinase like 5 (“CDKL5”) deficiency disorder, protocadherin19 (“PCDH19”) epilepsy, Lennox Gastaut Syndrome (“LGS”), Rett syndrome, and Fragile X Syndrome, Ohtahara syndrome, early myoclonic epileptic encephalopathy, West syndrome, Dravet syndrome, Angelman Syndrome, Continuous Spike Wave in Sleep (CSWS) epileptic syndrome and other diseases, e.g., X-linked myoclonic seizures, spasticity and intellectual disability syndrome, idiopathic infantile epileptic-dyskinetic encephalopathy, epilepsy and mental retardation limited to females, and severe infantile multifocal epilepsy. In some of these embodiments, the human has a low level of an endogenous neurosteroid(s) (e.g., allopregnanolone-sulfate (Allo-S)).

The invention is also directed to a method of treating a mammal with an epileptic encephalopathy, the method comprising orally administering to a mammal a solid oral immediate release formulation comprising a pharmaceutically acceptable pregnenolone neurosteroid (e.g., ganaxolone) on a twice-a-day basis (e.g., every 10-13 hours), wherein the neurosteroid has a half-life of from about 18 hours to about 24 hours, the formulation releases not less than about 70% or about 80% of ganaxolone at 45 minutes of placing the formulation into a simulated gastrointestinal fluid (SGF and/or SIF), and the administration results in at least about a 35%, about a 40%, about a 45%, or about a 50% decrease in seizure frequency per 28 days, as compared to the seizure frequency during a time period of 28 days before the first administration.

The invention is further directed to a method of treating a mammal with an epileptic encephalopathy, the method comprising orally administering to a mammal a liquid oral immediate release formulation comprising a pharmaceutically acceptable pregnenolone neurosteroid (e.g., ganaxolone) three times a day (e.g., every 6 to 8 hours), wherein the neurosteroid has a half-life of from about 18 hours to about 24 hours, the formulation releases not less than about 70% or about 80% of ganaxolone at 45 minutes of placing the formulation into a simulated gastrointestinal fluid (SGF and/or SIF) and the administration results in at least about a 35%, about a 40%, about a 45%, or about a 50% decrease in seizure frequency per 28 days, as compared to the seizure frequency during a time period of 28 days before the first administration.

The invention is also directed to a method for treating a patient with a pregnenolone neurosteroid, wherein the human is suffering from an encephalopathy, the method comprising the steps of:
determining whether the human has a low level of endogenous neurosteroid by:
   obtaining or having obtained a biological sample from the human; and
   performing or having performed an assay on the biological sample to determine the level of an endogenous neurosteroid(s),
wherein a level of the endogenous neurosteroid of 2500 pg mL⁻¹ or less, 2000 pg mL⁻¹ or less, 1500 pg mL⁻¹ or less, 1000 pg mL⁻¹ or less, 900 pg mL⁻¹ or less, 800 pg mL⁻¹ or less, 700 pg mL⁻¹ or less, 600 pg mL⁻¹ or less, 500 pg mL⁻¹ or less, 400 pg mL⁻¹ or less, 300 pg mL⁻¹ or less, 200 pg mL⁻¹ or less, 100 pg mL⁻¹ or less, 75 pg mL⁻¹ or less, 50 pg mL⁻¹ or less, or 25 pg mL⁻¹ or less indicates that the human has the low level of endogenous steroid,
and if the human has the low level of endogenous steroid orally administering a pregnenolone neurosteroid (e.g., ganaxolone) to the patient at a dose of from 1 mg/kg/day to about 63 mg/kg/day, from about 2 mg/kg/day to about 63 mg/kg/day, from about 3 mg/kg/day to about 63 mg/kg/day, from about 4 mg/kg/day to about 63 mg/kg/day, from about 5 mg/kg/day to about 63 mg/kg/day, from about 6 mg/kg/day to about 63 mg/kg/day, or from about 7 mg/kg/day to about 63 mg/kg/day for at least one day in two or three divided doses. In some of these embodiments, the level of endogenous neurosteroid of 2500 pg mL⁻¹ or less, 2000 pg mL⁻¹ or less, 1500 pg mL⁻¹ or less, 1000 pg mL⁻¹ or less, 900 pg mL⁻¹ or less, 800 pg mL⁻¹ or less, 700 pg mL⁻¹ or less, 600 pg mL⁻¹ or less, 500 pg mL⁻¹ or less, 400 pg mL⁻¹ or less, 300 pg mL⁻¹ or less, 200 pg mL⁻¹ or less, 100 pg mL⁻¹ or less, 75 pg mL⁻¹ or less, 50 pg mL⁻¹ or less, or 25 pg mL⁻¹ or less indicates that the administration of said ganaxolone is likely to reduce a seizure frequency in the patient, e.g., by 35%, or higher; about 40%, or higher; about 45%, or higher; or about 50%, or higher; after administration for 28 days, as compared to the seizure frequency during a time period of 28 days before the first administration. The endogenous neurosteroid may be selected from the group comprising or consisting of pregnanolone, pregnanolone-sulfate, 5-alphaDHP, allopregnanolone, allopregnanolone-S, pregnanolone, pregnanolone-S, DHEA, and combinations thereof, and the pregnenolone neurosteroid may, e.g., be selected from the group comprising or consisting of allopregnanolone, ganaxolone, alphaxalone, alphadolone, hydroxydione, minaxolone, pregnanolone, acebrochol, or tetrahydrocorticosterone, and pharmaceutically acceptable salts thereof. In some of these embodiments, the method further comprises communicating the results of the assay to the patient or a medical provider before or after the administration of the pregnenolone neurosteroid.

The invention is also directed to a method for treating a human with ganaxolone, wherein the human is suffering from an encephalopathy, the method comprising the steps of:
determining whether the human has a level of allopregnanolone-sulfate of 2500 pg mL⁻¹ or less,
and if the human has a level of allopregnanolone-sulfate of 2500 pg mL⁻¹ or less, then orally administering ganaxolone to the human at a dose of from 1 mg/kg/day to about 63 mg/kg/day, from about 2 mg/kg/day to about 63 mg/kg/day, from about 3 mg/kg/day to about 63 mg/kg/day, from about 4 mg/kg/day to about 63 mg/kg/day, from about 5 mg/kg/day to about 63 mg/kg/day, from about 6 mg/kg/day to about 63 mg/kg/day, or from about 7 mg/kg/day to about 63 mg/kg/day for at least one day in two or three divided doses. In some of these embodiments, the level of allopregnanolone-sulfate of 2500 pg mL⁻¹ or below indicates that the administration of said ganaxolone is likely to reduce a seizure frequency in the human, e.g., by at least about 35%, about 40%, about 45%, or about 50% after administration for 28 days, as compared to the seizure frequency during a time period of 28 days before the first administration.

The invention is further directed to a method for treating a human with ganaxolone, wherein the human is suffering from an encephalopathy, the method comprising the steps of:
determining whether the human has a level of allopregnanolone-sulfate of 2500 pg mL⁻¹ or less,
and if the human has a level of allopregnanolone-sulfate of 2500 pg mL⁻¹ or less, then orally administering an endogenous neurosteroid (e.g., allopregnanolone, pregnanolone, etc.) or a synthetic neurosteroid (e.g., Co26749/WAY-141839, Co134444, Co177843, Sage-217 (3α-Hydroxy-3β-methyl-21-(4-cyano-1H-pyrazol-1′-yl)-19-nor-50-pregnan-20-one), ganaxolone, etc.) to the human at a dose of from 1 mg/kg/day to about 63 mg/kg/day, from about 2 mg/kg/day to about 63 mg/kg/day, from about 3 mg/kg/day to about 63 mg/kg/day, from about 4 mg/kg/day to about 63 mg/kg/day, from about 5 mg/kg/day to about 63 mg/kg/day, from about 6 mg/kg/day to about 63 mg/kg/day, or from about 7 mg/kg/day to about 63 mg/kg/day for at least one day in two or three divided doses, and
if the human has a level of allopregnanolone-sulfate above 2500 pg mL⁻¹, refraining from administering the endogenous or synthetic neurosteroid to the human and/or administering a different anti-convulsant agent. A different anti-convulsant agent may, e.g., be selected from the group consisting of benzodiazepines (e.g., clobazam, diazepam, clonazepam, midazolam, etc.), clorazepic acid, levetiracetam, felbamate, lamotrigine, a fatty acid derivative (e.g., valproic acid), a carboxamide derivative (rufinamide, carbamazepine, oxcarbazepine, etc.), an amino acid derivative (e.g., levocarnitine), a barbiturate (e.g., phenobarbital), or a combination of two or more of the foregoing agents.

The invention is also directed to a method for treating a human with ganaxolone, wherein the human is suffering from an encephalopathy, the method comprising the steps of:
determining whether the human has a level of allopregnanolone-sulfate of 2500 pg mL⁻¹ or less,
and if the human has a level of allopregnanolone-sulfate of 2500 pg mL⁻¹ or less, then orally administering ganaxolone to the human at a dose of from 1 mg/kg/day to about 63 mg/kg/day, from about 2 mg/kg/day to about 63 mg/kg/day, from about 3 mg/kg/day to about 63 mg/kg/day, from about 4 mg/kg/day to about 63 mg/kg/day, from about 5 mg/kg/day to about 63 mg/kg/day, from about 6 mg/kg/day to about 63 mg/kg/day, or from about 7 mg/kg/day to about 63 mg/kg/day for at least one day in two or three divided doses. In some of these embodiments, the level of allopregnanolone-sulfate of 2500 pg mL⁻¹ or below indicates that the administration of said ganaxolone is likely to reduce a seizure frequency in the human, e.g., by at least about a 35%, about a 40%, about a 45%, or about a 50% after administration for 28 days, as compared to the seizure frequency during a time period of 28 days before the first administration.

The invention is further directed to a method for treating a human with ganaxolone, wherein the human is suffering from an encephalopathy, the method comprising the steps of:
determining whether the human has a level of allopregnanolone of 200 pg mL⁻¹ or less,
and if the human has a level of allopregnanolone of 200 pg mL⁻¹ or less, then orally administering ganaxolone to the human at a dose of from 1 mg/kg/day to about 63 mg/kg/day, from about 2 mg/kg/day to about 63 mg/kg/day, from about 3 mg/kg/day to about 63 mg/kg/day, from about 4 mg/kg/day to about 63 mg/kg/day, from about 5 mg/kg/day to about 63 mg/kg/day, from about 6 mg/kg/day to about 63 mg/kg/day, or from about 7 mg/kg/day to about 63 mg/kg/day for at least one day in two or three divided doses, and
if the human has a level of allopregnanolone above 200 pg mL⁻¹, refraining from administering ganaxolone to the human. In some of these embodiments, the level of allopregnanolone of 200 pg mL⁻¹ or below indicates that the administration of said ganaxolone is likely to reduce a seizure frequency in the human, e.g., by at least about a 35%, about a 40%, about a 45%, or about a 50% after administration for 28 days, as compared to the seizure frequency during a time period of 28 days before the first administration.

The invention is further directed to a method for treating a human with ganaxolone, wherein the human is suffering from an encephalopathy, the method comprising the steps of:
determining whether the human has a level of allopregnanolone of 200 pg mL⁻¹ or less, and
if the human has a level of allopregnanolone of 200 pg mL⁻¹ or less, then orally administering ganaxolone to the human at a dose of from 1 mg/kg/day to about 63 mg/kg/day, from about 2 mg/kg/day to about 63 mg/kg/day, from about 3 mg/kg/day to about 63 mg/kg/day, from about 4 mg/kg/day to about 63 mg/kg/day, from about 5 mg/kg/day to about 63 mg/kg/day, from about 6 mg/kg/day to about 63 mg/kg/day, or from about 7 mg/kg/day to about 63 mg/kg/day for at least one day in two or three divided doses, and
if the human has a level of allopregnanolone above 200 pg mL⁻¹, refraining from administering ganaxolone to the human.

The present invention is also directed to a method of treating an encephalopathy in a human comprising administering a pharmaceutically acceptable pregnenolone neurosteroid (e.g., ganaxolone) to the human at a dose of about 1800 mg, or less, per day, for at least 1 day, wherein the human has a genetic mutation in gene selected from the group consisting of ALDH7A1, KCNQ2, KCNQ3, TBC1D24, PRRT2, SCN2A, SCN8A, ST3GAL5, CACNA1A, GABRA1, GABRB3, KCNT1, AARS, ARV1, DOCK7, FRRS1L, GUF1, ITPA, NECAP1, PLCB1, SLC12A5, SLC13A5, SLC25A12, SLC25A22, ST3GAL3, SZT2, WWOX, CDKL5, ARHGEF9, ALG13, PCDH19, DNM1, EEF1A2, FGF12, GABRB1, GNAO1, GRIN2B, GRIN2D, HCN1, KCNA2, KCNB1, SIK1, SLC1A2, SPTAN1, STXBP1, UBA5, SCN1A, SCN9Ab, GPR98, SCN9A, CPA6, GABRD, GABRG2, SCN1B, STX1B, KCNMA1, SLC6A1, CHD2, GRIN2A, CACNA1H, CLCN2a, EFHC1, CACNB4, SLC2A1, CASR, ADRA2B, CNTN2, GAL, GI1, KCNC1, CERS1, CSTB, EPM2A, GOSR2, KCTD7, LMNB2, NHLRC1, PRDM8, PRICKLE1, SCARB2, CHRNA2, CHRNA4, CHRNB2, DEPDC5, UBE3A, MeCP2, TSC1, TSC2, FOXG1, TPP1, ZEB2, ARX, CHRNA7, TCF4, POLG, SLC9A6, MEF2C, MBD5, CLN3, CLN5, CLN6, ATP1A2, LG11, KANSL1, GAMT, CNTNAP2, KCNJ10, PNKP, PPT1, ADSL, MFSD8, SYN1, CLN8, ATP6AP2, CTSD, DNAJC5, FOLR1, GATM, GOSR2, LIAS, MAG12, NRXN1, SRPX2, and combinations of two or more of any of the foregoing, and at least one of the symptoms experienced by the human is selected from the group consisting of (i) uncontrolled cluster seizures (3 or more seizures over the course of 12 hours) during a time period of from 4 to 8 weeks (e.g., 6 weeks), (ii) bouts of status epilepticus on intermittent basis, the method, (iii) uncontrolled non-clustered seizures (focal dyscognitive, focal convulsive, atypical absences, hemiclonic seizures, spasms, or tonic-spasm seizures) with a frequency≥4 seizures during a time period of from 4 to 8 weeks (e.g., 4 weeks), (iv) ≥4 generalized convulsive (tonic-clonic, tonic, clonic, atonic seizures) seizures during a time period of 4 to 8 weeks (e.g., 4 weeks), and (v) a combinations of any two or more of the foregoing. In some of these embodiments, the pharmaceutically acceptable pregnenolone neurosteroid is ganaxolone and is administered orally in the amount of from about 200 mg/day to about 1800 mg/day, from about 300 mg/day to about 1800 mg/day, from about 400 mg/day to about 1800 mg/day, from about 450 mg/day to about 1800 mg/day, from about 675 mg/day to about 1800 mg/day, from about 900 mg/day to about 1800 mg/day, from about 1125 mg/day to about 1800 mg/day, from about 1350 mg/day to about 1800 mg/day, from about 1575 mg/day to about 1800 mg/day, or about 1800 mg/day, in two or three divided doses. In some embodiments, administration of the pharmaceutically acceptable pregnenolone neurosteroid results in a 35%, or better (e.g., about a 40%, about 45%, about 50%, about 55%) reduction in mean seizure frequency per 28 days, as compared to the seizure frequency during a time period of 28 days before the first administration. In some embodiments, the improvement is 50% or more.

The present invention is also directed to the treatment of human patients who have experienced an early onset infantile epileptic encephalopathy. Examples of such early onset infantile epileptic encephalopathies include but are not limited to Ohtahara syndrome, early myoclonic epileptic encephalopathy, West syndrome, Dravet syndrome, PCDH19 (protocadherin 19) epilepsy, CDKL5 (cyclin-dependent kinase-like 5) epilepsy, Lennox-Gastaut Syndrome (LGS), Continuous Spike and Wave During Sleep (CSWS) and other diseases, e.g., X-linked myoclonic seizures, spasticity and intellectual disability syndrome, idiopathic infantile epileptic-dyskinetic encephalopathy, epilepsy and mental retardation limited to females, and severe infantile multifocal epilepsy. The method comprises administering a pharmaceutically acceptable pregnenolone neurosteroid (e.g., ganaxolone) to the mammal at a dose of from about at a dose of from 1 mg/kg/day to about 63 mg/kg/day, provided that the total amount of administered ganaxolone does not exceed 1800 mg/day.

The invention is further directed to a method of treating a mammal (e.g., a human) having a history of (i) uncontrolled cluster seizures (3 or more seizures over the course of 12 hours) during a time period of from 4 to 8 weeks (e.g., 6 weeks) and/or (ii) bouts of status epilepticus on intermittent basis, the method and/or (iii) uncontrolled non-clustered seizures (focal dyscognitive, focal convulsive, atypical absences, hemiclonic seizures, spasms, or tonic-spasm seizures) with a frequency≥4 seizures during a time period of from 4 to 8 weeks (e.g., 4 weeks) and/or (iv) ≥4 generalized convulsive (tonic-clonic, tonic, clonic, atonic seizures) seizures during a time period of 4 to 8 weeks (e.g., 4 weeks), the method comprising administering a pharmaceutically acceptable pregnenolone neurosteroid (e.g., ganaxolone) to the mammal at a dose of from about at a dose of from 1 mg/kg/day to about 63 mg/kg/day, from about 2 mg/kg/day to about 63 mg/kg/day, from about 3 mg/kg/day to about 63 mg/kg/day, from about 4 mg/kg/day to about 63 mg/kg/day, from about 5 mg/kg/day to about 63 mg/kg/day, from about 6 mg/kg/day to about 63 mg/kg/day, or from about 7 mg/kg/day to about 63 mg/kg/day, provided that the total amount of administered ganaxolone does not exceed 1800 mg/day.

In an additional aspect, the invention is directed to a method of treating a mammal (e.g., human) having subclinical CSWS syndrome with or without clinical events on EEG, the method comprising administering a pharmaceutically acceptable pregnenolone neurosteroid (e.g., ganaxolone) to the mammal at a dose of from about at a dose of from 1 mg/kg/day to about 63 mg/kg/day, from about 2 mg/kg/day to about 63 mg/kg/day, from about 3 mg/kg/day to about 63 mg/kg/day, from about 4 mg/kg/day to about 63 mg/kg/day, from about 5 mg/kg/day to about 63 mg/kg/day, from about 6 mg/kg/day to about 63 mg/kg/day, or from about 7 mg/kg/day to about 63 mg/kg/day, provided that the total amount of administered ganaxolone does not exceed 1800 mg/day.

The present invention is directed in part to the use of pregnenolone neurosteroids such as ganaxolone in the treatment of gene-related early onset infantile epileptic encephalopathies such as PCDH19 female predominant epilepsy and CDKL5 deficiency disorder. Administration of the pregnenolone neurosteroid(s) in accordance with the present invention may help to compensate for the effects of allopregnanolone deficiency.

The invention is also directed to a method of treating a mammal (e.g., a human) with PCDH19 disorder, the method comprising administering a pharmaceutically acceptable pregnenolone neurosteroid (e.g., ganaxolone) to the mammal at a dose of from about at a dose of from 1 mg/kg/day to about 63 mg/kg/day, from about 2 mg/kg/day to about 63 mg/kg/day, from about 3 mg/kg/day to about 63 mg/kg/day, from about 4 mg/kg/day to about 63 mg/kg/day, from about 5 mg/kg/day to about 63 mg/kg/day, from about 6 mg/kg/day to about 63 mg/kg/day, or from about 7 mg/kg/day to about 63 mg/kg/day, provided that the total amount of administered ganaxolone does not exceed 1800 mg/day.

The invention is also directed to a method of treating a mammal (e.g., a human) with Dravet Syndrome, the method comprising administering a pharmaceutically acceptable pregnenolone neurosteroid (e.g., ganaxolone) to the mammal at a dose of from about at a dose of from 1 mg/kg/day to about 63 mg/kg/day, from about 2 mg/kg/day to about 63 mg/kg/day, from about 3 mg/kg/day to about 63 mg/kg/day, from about 4 mg/kg/day to about 63 mg/kg/day, from about 5 mg/kg/day to about 63 mg/kg/day, from about 6 mg/kg/day to about 63 mg/kg/day, or from about 7 mg/kg/day to about 63 mg/kg/day, provided that the total amount of administered ganaxolone does not exceed 1800 mg/day.

The invention is also directed to a method of treating a mammal with LGS, the method comprising administering a pharmaceutically acceptable pregnenolone neurosteroid (e.g., ganaxolone) to the mammal at a dose of from about at a dose of from 1 mg/kg/day to about 63 mg/kg/day, from about 2 mg/kg/day to about 63 mg/kg/day, from about 3 mg/kg/day to about 63 mg/kg/day, from about 4 mg/kg/day to about 63 mg/kg/day, from about 5 mg/kg/day to about 63 mg/kg/day, from about 6 mg/kg/day to about 63 mg/kg/day, or from about 7 mg/kg/day to about 63 mg/kg/day, provided that the total amount of administered ganaxolone does not exceed 1800 mg/day.

The invention is also directed to a method of treating a mammal with CSWS, the method comprising administering a pharmaceutically acceptable pregnenolone neurosteroid (e.g., ganaxolone) to the mammal at a dose of from about at a dose of from 1 mg/kg/day to about 63 mg/kg/day, from about 2 mg/kg/day to about 63 mg/kg/day, from about 3 mg/kg/day to about 63 mg/kg/day, from about 4 mg/kg/day to about 63 mg/kg/day, from about 5 mg/kg/day to about 63 mg/kg/day, from about 6 mg/kg/day to about 63 mg/kg/day, or from about 7 mg/kg/day to about 63 mg/kg/day, provided that the total amount of administered ganaxolone does not exceed 1800 mg/day.

In certain embodiments, the method of the invention further comprises periodic measurements of plasma levels of the administered a pharmaceutically acceptable pregnenolone neurosteroid and/or concomitant AED medication(s), if any, and/or allopregnanolone (3α-hydroxy-5α-pregnan-20-one) and/or related endogenous CNS-active steroids. In some embodiments, the plasma levels of liver enzymes (AST, ALT and ALK Phos) are also measured before, during or after initiation of treatment with the pharmaceutically acceptable pregnenolone neurosteroid. The plasma levels may, e.g., be measured weekly, every 2 weeks, every 3 weeks, every 4 weeks, every 5 weeks, every 6 weeks, every 7 weeks, every 8 weeks, every 9 weeks, every 10 weeks, every 11 week, or every 12 weeks.

In certain embodiments, the low endogenous level of neurosteroid can be measured in the human as a plasma allopregnanolone-sulfate of about 2500 pg/ml or less. Thus, the low endogenous level of neurosteroid in the human may, e.g., be 2400 pg/ml or less, 2300 pg/ml or less, 2200 pg/ml or less, 2100 pg/ml or less, 2000 pg/ml or less, 1900 pg/ml or less, 1800 pg/ml or less, 1700 pg/ml or less, 1600 pg/ml or less, 1500 pg/ml or less, 1400 pg/ml or less, 1300 pg/ml or less, 1200 pg/ml or less, 1100 pg/ml or less, 1000 pg/ml or less, 900 pg/ml or less, 850 pg/ml or less, 800 pg/ml or less, 750 pg/ml or less, 700 pg/ml or less, 650 pg/ml or less, 600 pg/ml or less, 550 pg/ml or less, 500 pg/ml or less, 450 pg/ml or less, 400 pg/ml or less, 350 pg/ml or less, 300 pg/ml or less, 250 pg/ml or less, 200 pg/ml or less, 1500 pg/ml or less, 100 pg/ml or less, 50 pg/ml or less, 25 pg/ml or less, 10 pg/ml or less, or 5 pg/ml or less.

In certain embodiments, the low endogenous level of neurosteroid can be measured in the human as a plasma allopregnanolone level of about 200 pg/ml or less. Thus, the low endogenous level of neurosteroid in the human may, e.g., be 200 pg/ml or less, 199 pg/ml or less, 198 pg/ml or less, 197 pg/ml or less, 196 pg/ml or less, 195 pg/ml or less, 194 pg/ml or less, 193 pg/ml or less, 192 pg/ml or less, 191 pg/ml or less, 190 pg/ml or less, 189 pg/ml or less, 188 pg/ml or less, 187 pg/ml or less, 186 pg/ml or less, 185 pg/ml or less, 184 pg/ml or less, 183 pg/ml or less, 182 pg/ml or less, 181 pg/ml or less, 180 pg/ml or less, 179 pg/ml or less, 178 pg/ml or less, 177 pg/ml or less, 176 pg/ml or less, 175 pg/ml or less, 174 pg/ml or less, 172 pg/ml or less, 171 pg/ml or less, 170 pg/ml or less, 169 pg/ml or less, 168 pg/ml or less, 167 pg/ml or less, 166 pg/ml or less, 165 pg/ml or less, 164 pg/ml or less, 163 pg/ml or less, 162 pg/ml or less, 161 pg/ml or less, 160 pg/ml or less, 159 pg/ml or less, 158 pg/ml or less, 157 pg/ml or less, 156 pg/ml or less, 155 pg/ml or less, 154 pg/ml or less, 153 pg/ml or less, 152 pg/ml or less, 151 pg/ml or less, 150 pg/ml or less, 149 pg/ml or less, 148 pg/ml or less, 147 pg/ml or less, 146 pg/ml or less, 145 pg/ml or less, 144 pg/ml or less, 143 pg/ml or less, 142 pg/ml or less, 141 pg/ml or less, 140 pg/ml or less, 139 pg/ml or less, 138 pg/ml or less, 137 pg/ml or less, 136 pg/ml or less, 135 pg/ml or less, 134 pg/ml or less, 133 pg/ml or less, 132 pg/ml or less, 131 pg/ml or less, 130 pg/ml or less, 129 pg/ml or less, 128 pg/ml or less, 127 pg/ml or less, 126 pg/ml or less, 125 pg/ml or less, 124 pg/ml or less, 123 pg/ml or less, 122 pg/ml or less, 121 pg/ml or less, 120 pg/ml or less, 119 pg/ml or less, 118 pg/ml or less, 117 pg/ml or less, 116 pg/ml or less, 115 pg/ml or less, 114 pg/ml or less, 113 pg/ml or less, 112 pg/ml or less, 111 pg/ml or less, 110 pg/ml or less, 109 pg/ml or less, 108 pg/ml or less, 107 pg/ml or less, 106 pg/ml or less, 105 pg/ml or less, 104 pg/ml or less, 103 pg/ml or less, 102 pg/ml or less, 101 pg/ml or less, 100 pg/ml or less, 99 pg/ml or less, 98 pg/ml or less, 97 pg/ml or less, 96 pg/ml or less, 95 pg/ml or less, 94 pg/ml or less, 93 pg/ml or less, 92 pg/ml or less, 91 pg/ml or less, 90 pg/ml or less, 89 pg/ml or less, 88 pg/ml or less, 87 pg/ml or less, 86 pg/ml or less, 85 pg/ml or less, 84 pg/ml or less, 83 pg/ml or less, 82 pg/ml or less, 81 pg/ml or less, 80 pg/ml or less, 79 pg/ml or less, 78 pg/ml or less, 77 pg/ml or less, 76 pg/ml or less, 75 pg/ml or less, 74 pg/ml or less, 73 pg/ml or less, 72 pg/ml or less, 71 pg/ml or less, 70 pg/ml or less, 69 pg/ml or less, 68 pg/ml or less, 67 pg/ml or less, 66 pg/ml or less, 65 pg/ml or less, 64 pg/ml or less, 63 pg/ml or less, 62 pg/ml or less, 61 pg/ml or less, 60 pg/ml or less, 59 pg/ml or less, 58 pg/ml or less, 57 pg/ml or less, 56 pg/ml or less, 55 pg/ml or less, 54 pg/ml or less, 53 pg/ml or less, 52 pg/ml or less, 51 pg/ml or less, 50 pg/ml or less, 49 pg/ml or less, 48 pg/ml or less, 47 pg/ml or less, 46 pg/ml or less, 45 pg/ml or less, 44 pg/ml or less, 43 pg/ml or less, 42 pg/ml or less, 41 pg/ml or less, 40 pg/ml or less, 39 pg/ml or less, 38 pg/ml or less, 37 pg/ml or less, 36 pg/ml or less, 35 pg/ml or less, 34 pg/ml or less, 33 pg/ml or less, 32 pg/ml or less, 31 pg/ml or less, 30 pg/ml or less, 29 pg/ml or less, 28 pg/ml or less, 27 pg/ml or less, 26 pg/ml or less, 25 pg/ml or less, 24 pg/ml or less, 23 pg/ml or less, 22 pg/ml or less, 21 pg/ml or less, 20 pg/ml or less, 19 pg/ml or less, 18 pg/ml or less, 17 pg/ml or less, 16 pg/ml or less, 15 pg/ml or less, 14 pg/ml or less, 13 pg/ml or less, 12 pg/ml or less, 11 pg/ml or less, 10 pg/ml or less, 9 pg/ml or less, 8 pg/ml or less, 7 pg/ml or less, 6 pg/ml or less, 5 pg/ml or less, 4 pg/ml or less, 3 pg/ml or less, 2 pg/ml or less, 1 pg/ml or less, or 0 pg/ml.

The pregnenolone neurosteroid may preferably be administered orally or parenterally. In certain preferred embodiments, the pregnenolone neurosteroid is ganaxolone and is administered as an oral suspension or an oral solid dosage form (e.g., oral capsule) at a dose of up to a total of 63 mg/kg/day, and ganaxolone is preferably administered up to a maximum amount of 1800 mg/day. Preferably, ganaxolone is administered chronically, e.g., for as long as the patient receives a therapeutic benefit from the treatment without untoward side effects requiring discontinuation of treatment. In certain embodiments, ganaxolone is administered for at least one day, at least 2 days, at least 3 days, 2 weeks, at least 3 weeks, at least 4 weeks, at least 5 weeks, at least 6 weeks, at least 7 weeks, at least 8 weeks, at least 9 weeks, at least 10 weeks, at least 11 weeks or at least 12 weeks.

When the pregnenolone neurosteroid is administered in an oral suspension, it may be administered, e.g., anywhere from one to about three times per day. In certain preferred embodiments, when the pregnenolone neurosteroid (e.g., ganaxolone) is orally administered, it may be administered with food (for better absorption) or without food. When the pregnenolone neurosteroid is administered in an oral tablet or capsule, it may be administered, e.g., anywhere from one to about four times per day. When the pregnenolone neurosteroid is administered parenterally, it may be administered, e.g., anywhere from one to about three times per day.

The invention is further directed to a method of treating a gene-related early onset infantile epileptic encephalopathy, comprising identifying a human patient suffering from a gene-related early-onset infantile epileptic encephalopathy, determining if that human patient has a low endogenous level of a neurosteroid(s), and administering the human patient a dosage regimen of a pharmaceutically acceptable pregnenolone neurosteroid (e.g., ganaxolone) in an amount effective to reduce the frequency of seizures in the human patient. The low level of an endogeneous neurosteroid may, e.g., be a level of allopregnanolone-sulfate of 2500 pg mL⁻¹ or below, and/or a level of allopregnanolone of 200 mg mL⁻¹ or below. In certain embodiments, the gene-related early-onset infantile epileptic encephalopathy is selected from, e.g., CDKL5 deficiency disorder, PCDH19 epilepsy, Lennox Gastaut Syndrome, Rett syndrome, Fragile X Syndrome, Ohtahara syndrome, early myoclonic epileptic encephalopathy, West syndrome, Dravet syndrome, and other diseases, e.g., X-linked myoclonic seizures, spasticity and intellectual disability syndrome, idiopathic infantile epileptic-dyskinetic encephalopathy, epilepsy and mental retardation limited to females, and severe infantile multifocal epilepsy. In certain embodiments, the gene-related early onset infantile epileptic encephalopathy is CDKL5, and the patients have a CDKL5 genetic mutation.

The invention is also directed to a method of treating a genetic epileptic encephalopathy condition or syndrome comprising testing whether a subject has a PCDH19 genetic mutation and/or CDKL5 genetic mutation and/or a SCN1A mutation, and, if the subject has the PCDH19 genetic mutation and/or the CDKL5 genetic mutation and/or the SCN1A mutation, administering a therapeutically effective amount of pregnenolone neurosteroid (e.g., ganaxolone) to the subject on a chronic basis. The method encompasses a step of communicating the results of the genetic testing to the subject and/or a medical provider after said testing and/or before said administration.

The invention is also directed to a method of treating a genetic epileptic encephalopathy condition or syndrome comprising ascertaining whether the subject has more than one type of generalized seizures, including, e.g., drop seizures (atonic, tonic, or myoclonic) for at least 6 months and an EEG pattern reporting diagnostic criteria for LGS at some point in their history (abnormal background activity accompanied by slow, spike, and wave pattern<2.5 Hz), and, if the subject does, administering a therapeutically effective amount of pregnenolone neurosteroid (e.g., ganaxolone) to the subject on a chronic basis. The method encompasses a step of communicating the results of the genetic testing to the subject and/or a medical provider after said testing and before said administration.

The invention is also directed to a method of treating a genetic epileptic encephalopathy condition or syndrome comprising ascertaining whether the subject has a current or historical EEG during sleep consistent with diagnosis of CSWS (e.g., continuous [85% to 100%] mainly bisynchronous 1.5 to 2 Hz [and 3 to 4 Hz] spikes and waves during non-REM sleep, and, if the subject does, subsequently administering a therapeutically effective amount of pregnenolone neurosteroid (e.g., ganaxolone) to the subject on a chronic basis. The method encompasses a step of communicating the results of the genetic testing to the subject and/or medical provider after said testing and before said administration.

The invention is also directed to a method of treating a genetic epileptic encephalopathy condition or syndrome comprising ascertaining whether the subject have had a prior positive response to response to administration of a steroid or ACTH, and, if the subject does, subsequently administering a therapeutically effective amount of pregnenolone neurosteroid (e.g., ganaxolone) to the subject on a chronic basis. The method encompasses a step of communicating the results of the genetic testing to the subject and/or medical provider after said testing and before said administration.

### Definitions

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely for illustration and does not pose a limitation on the scope of the invention unless otherwise claimed. No language in the specification should be construed as indicating any non-claimed element as essential to the practice of the invention.

The terms “a” and “an” do not denote a limitation of quantity, but rather denote the presence of at least one of the referenced item.

The term “about” is used synonymously with the term “approximately.” As one of ordinary skill in the art would understand, the exact boundary of “about” will depend on the component of the composition. Illustratively, the use of the term “about” indicates that values slightly outside the cited values, i.e., plus or minus 0.1% to 10%, which are also effective and safe. Thus compositions slightly outside the cited ranges are also encompassed by the scope of the present claims.

An “active agent” is any compound, element, or mixture that when administered to a patient alone or in combination with another agent confers, directly or indirectly, a physiological effect on the patient. When the active agent is a compound, salts, solvates (including hydrates) of the free compound or salt, crystalline and non-crystalline forms, as well as various polymorphs of the compound are included. Compounds may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers in pure form and mixtures thereof are encompassed. In addition, compounds with carbon-carbon double bonds may occur in Z- and E-forms, with all isomeric forms of the compounds being included in the present invention. In these situations, the single enantiomers, i.e. optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

The term “endogenous neurosteroid” means a steroid produced within the brain and capable of modulating neuronal excitability by interaction with neuronal membrane receptors and ion channels, principally GABA-A receptors, and includes, e.g., pregnane neurosteroids (e.g., allopregnanolone, allotetrahydrodeoxycorticosterone, etc.), androstane neurosteroids (e.g., androstanediol, etiocholanone, etc.), and sulfated neurosteroids (e.g., pregnanolone sulfate, dehydroepiandrosterone sulfate (DHEAS)).

The term “pregnenolone neurosteroid” means an endogenous or exogenous steroid capable of modulating neuronal excitability by interaction with neuronal membrane receptors and ion channels, principally GABA-A receptors, and encompasses, e.g., endogenous neurosteroids and synthetic neurosteroids synthesized or derived from pregnenolone in vitro and in vivo.

The term “biomarker” means a serum or plasma level of a neurosteroid that differentiates a drug responder from a non-responder.

The terms “serum” and “plasma” as disclosed herein may be used interchangeably.

The terms “comprising,” “including,” and “containing” are non-limiting. Other non-recited elements may be present in embodiments claimed by these transitional phrases. Where “comprising,” “containing,” or “including” are used as transitional phrases other elements may be included and still form an embodiment within the scope of the claim. The open-ended transitional phrase “comprising” encompasses the intermediate transitional phrase “consisting essentially of” and the close-ended phrase “consisting of.”

A “bolus dose” is a relatively large dose of medication administered in a short period, for example within 1 to 30 minutes.

“Cₘₐₓ” is the concentration of an active agent in the plasma at the point of maximum concentration.

“Ganaxolone” is also known as 3α-hydroxy-5α-pregnan-20-one, and is alternatively referred to as “GNX” in this document.

“Infusion” administration is a non-oral administration, typically intravenous though other non-oral routes such as epidural administration are included in some embodiments. Infusion administration occurs over a longer period than a bolus administration, for example over a period of at least 15 minutes, at least 30 minutes, at least 1 hour, at least 2 hours, at least 3 hours, or at least 4 hours.

A “patient” is a human or non-human animal in need of medical treatment. Medical treatment includes treatment of an existing condition, such as a disorder or injury. In certain embodiments treatment also includes prophylactic or preventative treatment, or diagnostic treatment.

A “child” means a human from 1 day to 18 years old (e.g., from 1 day to 15 years old), including 18 years old.

An “adult” means a human that is older than 18 years old.

“Pharmaceutical compositions” are compositions comprising at least one active agent, such as a compound or salt, solvate, or hydrate of Formula (I), and at least one other substance, such as a carrier. Pharmaceutical compositions optionally contain one or more additional active agents. When specified, pharmaceutical compositions meet the U.S. FDA's GMP (good manufacturing practice) standards for human or non-human drugs. “Pharmaceutical combinations” are combinations of at least two active agents which may be combined in a single dosage form or provided together in separate dosage forms with instructions that the active agents are to be used together to treat a disorder, such as a seizure disorder.

“Povidone” also known as polyvidone and polyvinylpyrrolidone (PVP) is a water soluble polymer made from the monomer, N-vinylpyrrolidone. Plasdone C-12 and C-17 are pharmaceutical grade homopolymers of N-vinylpyrrolidone. Plasdone C-12 has a K value of 10-2-13.8 and nominal molecular weight of 4000 d. Plasdone C-17 has a K-value of 15.5-17.5 and nominal molecular weight of 10,000 d.

“Sterilize” means to inactivate substantially all biological contaminates in a sample, formulation, or product. A 1-million fold reduction in the bioburden is also considered “sterilized” for most pharmaceutical applications.

The term “reduce” seizure or seizure activity refer to the detectable decrease in the frequency, severity and/or duration of seizures. A reduction in the frequency, severity and/or duration of seizures can be measured by self-assessment (e.g., by reporting of the patient) or by a trained clinical observer. Determination of a reduction of the frequency, severity and/or duration of seizures can be made by comparing patient status before and after treatment.

A “therapeutically effective amount” or “effective amount” is that amount of a pharmaceutical agent to achieve a pharmacological effect. The term “therapeutically effective amount” includes, for example, a prophylactically effective amount. An “effective amount” of neurosteroid is an amount needed to achieve a desired pharmacologic effect or therapeutic improvement without undue adverse side effects. The effective amount of neurosteroid will be selected by those skilled in the art depending on the particular patient and the disease. It is understood that “an effective amount” or “a therapeutically effective amount” can vary from subject to subject, due to variation in metabolism of neurosteroid, age, weight, general condition of the subject, the condition being treated, the severity of the condition being treated, and the judgment of the prescribing physician.

“Treat” or “treatment” refers to any treatment of a disorder or disease, such as inhibiting the disorder or disease, e.g., arresting the development of the disorder or disease, relieving the disorder or disease, causing regression of the disorder or disease, relieving a condition caused by the disease or disorder, or reducing the symptoms of the disease or disorder.

“Alkyl” is a branched or straight chain saturated aliphatic hydrocarbon group, having the specified number of carbon atoms, generally from 1 to about 8 carbon atoms. The term C₁-C₆-alkyl as used herein indicates an alkyl group having from 1, 2, 3, 4, 5, or 6 carbon atoms. Other embodiments include alkyl groups having from 1 to 6 carbon atoms, 1 to 4 carbon atoms or 1 or 2 carbon atoms, e.g. C₁-C₈-alkyl, C₁-C₄-alkyl, and C₁-C₂-alkyl. Examples of alkyl include, but are not limited to, methyl, ethyl, n-propyl, isopropyl, n-butyl, 3-methylbutyl, t-butyl, n-pentyl, and sec-pentyl.

“Aryl” indicates aromatic groups containing only carbon in the aromatic ring or rings. Typical aryl groups contain 1 to 3 separate, fused, or pendant rings and from 6 to about 18 ring atoms, without heteroatoms as ring members. When indicated, such aryl groups may be further substituted with carbon or non-carbon atoms or groups. Aryl groups include, for example, phenyl, naphthyl, including 1-naphthyl, 2-naphthyl, and bi-phenyl. An “arylalkyl” substituent group is an aryl group as defined herein, attached to the group it substitutes via an alkylene linker. The alkylene is an alkyl group as described herein except that it is bivalent.

“Cycloalkyl” is a saturated hydrocarbon ring group, having the specified number of carbon atoms. Monocyclic cycloalkyl groups typically have from 3 to about 8 carbon ring atoms or from 3 to 6 (3, 4, 5, or 6) carbon ring atoms. Cycloalkyl substituents may be pendant from a substituted nitrogen, oxygen, or carbon atom, or a substituted carbon atom that may have two substituents may have a cycloalkyl group, which is attached as a spiro group. Examples of cycloalkyl groups include cyclopropyl, cyclobutyl, cyclopentyl, and cyclohexyl.

A “heteroalkyl” group is an alkyl group as described with at least one carbon replaced by a heteroatom, e.g. N, O, or S.

The term “substituted” as used herein, means that any one or more hydrogens on the designated atom or group is replaced with a selection from the indicated group, provided that the designated atom's normal valence is not exceeded. When the substituent is oxo (i.e., ═O) then 2 hydrogens on the atom are replaced. When an oxo group substitutes a heteroaromatic moiety, the resulting molecule can sometimes adopt tautomeric forms. For example a pyridyl group substituted by oxo at the 2- or 4-position can sometimes be written as a pyridine or hydroxypyridine. Combinations of substituents and/or variables are permissible only if such combinations result in stable compounds or useful synthetic intermediates. A stable compound or stable structure is meant to imply a compound that is sufficiently robust to survive isolation from a reaction mixture and subsequent formulation into an effective therapeutic agent. Unless otherwise specified, substituents are named into the core structure. For example, it is to be understood that aminoalkyl means the point of attachment of this substituent to the core structure is in the alkyl portion and alkylamino means the point of attachment is a bond to the nitrogen of the amino group.

Suitable groups that may be present on a “substituted” or “optionally substituted” position include, but are not limited to, e.g., halogen; cyano; —OH; oxo; —NH₂; nitro; azido; alkanoyl (such as a C₂-C₆ alkanoyl group); C(O)NH₂; alkyl groups (including cycloalkyl and (cycloalkyl)alkyl groups) having 1 to about 8 carbon atoms, or 1 to about 6 carbon atoms; alkenyl and alkynyl groups including groups having one or more unsaturated linkages and from 2 to about 8, or 2 to about 6 carbon atoms; alkoxy groups having one or more oxygen linkages and from 1 to about 8, or from 1 to about 6 carbon atoms; aryloxy such as phenoxy; alkylthio groups including those having one or more thioether linkages and from 1 to about 8 carbon atoms, or from 1 to about 6 carbon atoms; alkylsulfinyl groups including those having one or more sulfinyl linkages and from 1 to about 8 carbon atoms, or from 1 to about 6 carbon atoms; alkylsulfonyl groups including those having one or more sulfonyl linkages and from 1 to about 8 carbon atoms, or from 1 to about 6 carbon atoms; aminoalkyl groups including groups having one or more N atoms and from 1 to about 8, or from 1 to about 6 carbon atoms; mono- or dialkylamino groups including groups having alkyl groups from 1 to about 6 carbon atoms; mono- or dialkylaminocarbonyl groups (i.e. alkylNHCO— or (alkyl1)(alkyl2)NCO—) having alkyl groups from about 1 to about 6 carbon atoms; aryl having 6 or more carbons.

“AARS” means alanyl-tRNA synthetase.

“ADRA2B” means alpha-2B-adrenergic receptor.

“ALDH7A1” means aldehyde dehydrogenase 7 family, member A1.

“ALG13” means asparagine-linked glycosylation 13, S. cerevisiae, homolog of.

“ARHGEF9” means RHO guanine nucleotide exchange factor 9.

“ARV1” means ARV1, S. cerevisiae, homolog of.

“CACNA1A” means calcium channel, voltage-dependent, P/Q type, alpha-1A subunit.

“CACNA1H” means calcium channel, voltage-dependent, T type, alpha-1H subunit.

“CACNB4” means calcium channel, voltage-dependent, beta-4 subunit.

“CASR” means calcium-sensing receptor.

“CDKL5” means cyclin-dependent kinase-like 5.

“CERS1” means ceramide synthase 1.

“CHD2” means chromodomain helicase DNA-binding protein 2.

“CHRNA2” means cholinergic receptor, neuronal nicotinic, alpha polypeptide 2.

“CHRNA4” means cholinergic receptor, neuronal nicotinic, alpha polypeptide 4.

“CHRNB2” means cholinergic receptor, neuronal nicotinic, beta polypeptide 2.

“CLCN2” means chloride channel 2; CNTN2, contactin 2.

“CPA6” means carboxypeptidase A6; CSTB, cystatin B.

“DEPDC5” means DEP domain-containing protein 5.

“DNM1” means dynamin 1.

“DOCK7” means dedicator of cytokinesis 7.

“EEF1A2” means eukaryotic translation elongation factor 1, alpha-2.

“EFHC1” means EF-hand domain (C-terminal)-containing protein 1.

“EPM2A” means EPM2A gene, encodes laforin.

“FGF12” means fibroblast growth factor 12.

“FRRS1L” means ferric chelate reductase 1-like.

“GABRA1” means gamma-aminobutyric acid receptor, alpha-1.

“GABRB1” means gamma-aminobutyric acid receptor, beta-1.

“GABRB3” means gamma-aminobutyric acid receptor, beta-3.

“GABRD” means gamma-aminobutyric acid receptor, delta.

“GABRG2” means gamma-aminobutyric acid receptor, gamma-2.

“GAL” means galanin; GNAO1, guanine nucleotide-binding protein, alpha-activating activity polypeptide O.

“GOSR2” means golgi snap receptor complex member 2.

“GPR98” means G protein-coupled receptor 98.

“GRIN2A” means glutamate receptor, ionotropic, N-methyl-D-aspartate, subunit 2A.

“GRIN2B” means glutamate receptor, ionotropic, N-methyl-D-aspartate, subunit 2B.

“GRIN2D” means glutamate receptor, ionotropic, N-methyl-D-aspartate, subunit 2D.

“GUF1” means GUF1 GTPase, S. cerevisiae, homolog of.

“HCN1” means hyperpolarization-activated cyclic nucleotide-gated potassium channel 1.

“ITPA” means inosine triphosphatase.

“KCNA2” means potassium channel, voltage-gated, shaker-related subfamily, member 2.

“KCNB1” means potassium channel, voltage-gated, shab-related subfamily, member 1.

“KCNC1” means potassium channel, voltage-gated, shaw-related subfamily, member 1.

“KCNMA1” means potassium channel, calcium-activated, large conductance, subfamily M, alpha member 1.

“KCNQ2” means potassium channel, voltage-gated, KQT-like subfamily, member 2.

“KCNQ3” means potassium channel, voltage-gated, KQT-like subfamily, member 3.

“KCNT1” means potassium channel, subfamily T, member 1.

“KCTD7” means potassium channel tetramerization domain-containing protein 7.

“LGI1” means leucine-rich gene, glioma-inactivated, 1.

“LMNB2” means lamin B2.

“NECAP1” means NECAP endocytosis-associated protein 1.

“NHLRC1” means NHL repeat-containing 1 gene.

“PCDH19” means protocadherin 19.

“PLCB1” means phospholipase C, beta-1.

“PNPO” means pyridoxamine 5-prime-phosphate oxidase.

“PRDM8” means PR domain-containing protein 8.

“PRICKLE1” means prickle, drosophila, homolog of, 1.

“PRRT2” means proline-rich transmembrane protein 2.

“SCARB2” means scavenger receptor class B, member 2.

“SCN1A” sodium channel, neuronal type I, alpha subunit.

“SCN1B” means sodium channel, voltage-gated, type I, beta subunit.

“SCN2A” means sodium channel, voltage-gated, type II, alpha subunit.

“SCN8A” means sodium channel, voltage-gated, type VIII, alpha subunit.

“SCN9A” means sodium channel, voltage-gated, type IX, alpha subunit.

“SIK1” means salt-inducible kinase 1.

“SLC1A2” means solute carrier family 1 (glial high affinity glutamate transporter), member 2.

“SLC12A5” means solute carrier family 12 (potassium/chloride transporter), member 5.

“SLC13A5” means solute carrier family 13 (sodium-dependent citrate transporter), member 5.

“SLC25A12” means solute carrier family 25 (mitochondrial carrier, aralar), member 12.

“SLC25A22” means solute carrier family 25 (mitochondrial carrier, glutamate), member 22.

“SLC2A1” means solute carrier family 2 (facilitated glucose transporter), member 1.

“SLC6A1” means solute carrier family 6 (neurotransmitter transporter, gaba), member 1.

“SPTAN1” means spectrin, alpha, nonerythrocytic 1.

“ST3GAL3” means ST3 beta-galactoside alpha-2,3-sialyltransferase 3.

“ST3GAL5” means ST3 beta-galactoside alpha-2,3-sialyltransferase 5.

“STX1B” means syntaxin 1B.

“STXBP1” means syntaxin-binding protein 1.

“SZT2” means seizure threshold 2, mouse, homolog of.

“TBC1D24” means Tre2-Bub2-Cdc16/TBC1 domain family, member 24.

“UBA5” means ubiquitin-like modifier activating enzyme 5.

“WWOX” means WW domain-containing oxidoreductase.

### BRIEF DESCRIPTION OF THE DRAWINGS

FIG. 1 is a diagram depicting effectiveness of AEDs after 12 months of use in PCDH19 patients. Abbreviations can be found in Lotte et al, 2016, herein incorporated by reference.

FIG. 2 is a graphical representation of the particle size data from the manufacture of ganaxolone nanomilled dispersion, bulk IR beads and encapsulated IR Beads. A typical decrease in particle size during milling, followed by particle size growth following addition of stabilizers during the curing period and a plateau achieved at approximately 300 nm.

FIG. 3A provides a summary of the key steps in manufacturing processes for manufacturing 50 mg/ml suspension and 225 mg capsules comprising IR release ganaxolone particles. As shown, both products utilize a common stabilized dispersion intermediate.

FIG. 3B is a summary of the key steps in the suspension manufacturing process that apply to the 50 mg/ml ganaxolone suspension 50 mg/ml of Example 1.

FIG. 3C is a summary of the key steps in the manufacturing process that apply to the 225 mg ganaxolone capsules of Example 2.

FIG. 3D is a graph of particle size stability of ganaxolone nanomilled suspension and encapsulated IR beads.

FIG. 3E is a graph of curing curve of ganaxolone particles containing parabens. The stabilized 300 nm nanoparticles exhibit good stability against particle growth in pediatric suspension drug product and encapsulated drug product formats. The stabilization process is controlled by accurate addition and dissolution of parabens, which are water soluble stabilization agents. The curing process is controlled by regulation of hold time and temperature of the stabilized dispersion prior to suspension dilution (in the case of 50 mg/ml ganaxolone suspension) or fluid bed bead coating (in the case of 225 mg ganaxolone capsule).

FIG. 4 presents the cumulative responder curve in terms of the 28-day seizure frequency for the sum of individual seizures and clusters of Example 4.

FIG. 5 is mean ganaxolone plasma concentration profile following a single oral dose of ganaxolone 0.3 micron capsules of Example 2 in healthy volunteers after a high fat meal (Example 5).

FIG. 6 is ganaxolone mean plasma concentration-time profiles following single and multiple BID oral doses of 0.3 micron ganaxolone capsules of Example 2 with a standard meal or snack in healthy volunteers (Example 5).

FIG. 7 is ganaxolone mean plasma concentration-time profiles following single and multiple BID oral doses of 0.3 micron ganaxolone capsules with a standard meal or snack in healthy volunteers.

FIG. 8 is ganaxolone mean plasma concentration-time profiles following multiple BID oral doses of 0.3 micron ganaxolone capsules with a standard meal or snack in healthy volunteers.

FIG. 9 is ganaxolone mean plasma trough levels following multiple BID oral doses of 0.3 micron ganaxolone capsules with a standard meal or snack—semilogarithmic axes. Subjects received 600 mg ganaxolone BID on Days 4-6; 800 mg ganaxolone BID on Days 7-9; and 1000 mg ganaxolone BID on Days 10-12. Values at Day 6.5, 9.5 and 12.5 are from evening samples collected 12 hrs after the last dose on PK sampling days.

FIG. 10 is plasma Allo-S concentration (pg mL⁻¹) in responders and non-responders of Example 11.

FIG. 11 is stratification of PCDH19 subjects by allopregnanolone sulfate (Allo-S) levels and the associated seizure-frequency response to ganaxolone in Example 11. “−100 change” means complete seizure freedom, patient not experiencing any seizures during that 26 week period. Anywhere between “0” and “−100%” is showing efficacy. The circles indicate “Responders” (≥25% reduction in seizure-frequency), and the squires indicate “Non-responders” (<25%) reduction in seizure-frequency.

FIG. 12 is stratification of CDKL5 subjects by allopregnanolone (Allo) level and associated seizure frequency response to ganaxolone in Example 11. Each closed circle represents a unique subject in the trial.

FIG. 13 shows relationship between dose and exposure (AUC) of ganaxolone in 0.3 micron capsule formulation showing saturation of exposure as doses approach 2000 mg/day.

### DETAILED DESCRIPTION

### CDKL5

CDKL5 Deficiency Disorder or CDKL5 stands for cyclin dependent kinase like 5.

CDKL5 gene is located on the X chromosome and was previously called STK9.

Most of the children affected by CDKL5 present with irritability in the perinatal period, early epilepsy, hand stereotypies, severely impaired psychomotor development and severe hypotonia. In contrast to classical Rett syndrome they also may have absence of a classic regression period, poor eye contact, generally normal head circumference and other growth parameters and relative absence of autonomic dysfunction.

Other symptoms of a CDKL5 Deficiency Disorder often include: low muscle tone, hand wringing movements or mouthing of the hands, marked developmental delay, limited or absent speech, lack of eye contact or poor eye contact, gastroesophageal reflux, constipation, small, cold feet, breathing irregularities such as hyperventilation, grinding of the teeth, episodes of laughing or crying for no reason, low/Poor muscle tone, very limited hand skills, some autistic-like tendencies, scoliosis, Cortical Visual Impairment (CVI), aka “cortical blindness”, apraxia, eating/drinking challenges, sleep difficulties and characteristics such as a sideways glance, and habit of crossing leg.

CDKL5 deficiency disorder is among the genetic epilepsies with encephalopathy that are virtually always refractory to treatment.

Seizures begin within the first days to months of life and become progressively more difficult to treat in most patients. The best initial response to therapeutic agents other than neurosteroid is to valproic acid, but at 12 months the responder rate is only 9% (Mueller et al). Commonly used AEDs for treatment of CDKL5 deficiency disorder include vigabatrin, felbamate and valproic acid. All 3 of these AEDs are associated with significant side effects. In addition to the risk of visual field loss with vigabatrin and aplastic anemia with felbamate, the tolerability of these 3 drugs is relatively low, particularly for long-term treatment. Patients may also be treated with high dose pulse steroids or ACTH, neither of which can be given long term due to frequent and severe side effects. Vagal nerve stimulation and corpus callosotomy are tried in very hopeless cases, both of which are invasive and are not generally effective. Corpus callosotomy is particularly invasive and only provides temporary relief from generalized seizures, and only in some cases. Unlike ganaxolone, which may improve cognitive and motor function, many available AEDs have side effects including cognitive dulling, ataxia, hepatotoxicity, and serious weight management problems—none of which have been associated with use of ganaxolone. Frequent monitoring of blood levels of ganaxolone are also not required, in contrast to narrow therapeutic index drugs such as the sodium channel blockers, phenytoin and carbamazepine.

CDKL5 was identified through an exon-trapping method designed to screen candidate genes in Xp22, a chromosome X region where several other genetic disorders have been mapped (Montini et al, 1998). CDKL5 is a member of a proline-directed kinase subfamily that has homology to both cell-cycle dependent kinases known as the CDKL kinases and microtubule-associated proteins (MAP) (Lin et al, 2005; Guerrini and Parrini, 2012).

The human CDKL5 gene occupies approximately 240 kb of the Xp22 region and is composed of 24 exons of which the first 3 (exons 1, 1a, 1b) are untranslated, whereas the coding sequences are contained within exons 2-21. Two splice variants with distinct 5′ untranslated region (5′ UTR) (also known as a Leader Sequence or Leader RNA) have been found: isoform I, containing exon 1, is transcribed in a wide range of tissues, whereas the expression of isoform II, including exons 1a and 1b, is limited to testis and fetal brain. Alternative splicing events lead to at least 3 distinct human protein isoforms. The original CDKL5 transcript generates a protein of 1030 amino acids (CDKL5-115; 115 kDa). While CDKL5-115 is expressed mainly in the testis, recently identified transcripts are likely to be relevant for CDKL5 brain functions characterized by an altered C-terminal region. Such differential enrichment of the CDKL5 splice variants by organ suggest that the alternative splicing is involved in regulating the protein functions. CDKL5 is a ubiquitous protein but is expressed mainly in the brain (cerebral cortex, hippocampus, cerebellum, striatum, and brainstem), thymus, and testes (Lin et al, 2005).

CDKL5 is a protein whose gene is located on the X chromosome. The CDKL5 gene provides instructions for making a protein that is essential in forming the connections for normal brain development, with mutations causing a deficiency in the protein level. CDKL5 deficiency disorder syndrome is characterized by early-onset intractable seizures, severely impaired gross motor skills and global developmental delay with sleep disturbances, abnormal muscle tone, bruxism, scoliosis, and gastrointestinal issues (Mangatt M, Wong K, Anderson B, Epstein A, Hodgetts S, Leonard H. Downs J. Prevalence and onset of comorbidities in the CDKL5 Deficiency Disorder differ from Rett syndrome. Orphanet Journal of Rare Diseases. 2016; 11:39).

Kalscheuer et al. (2003) reported on 2 non-related girls who presented with infantile spasms (diagnosed at that time as West Syndrome) and profound developmental delay. In both patients, the CDKL5 gene was disrupted by a breakpoint on the X chromosome due to a balanced translocation. The overlapping clinical similarities between these first patients and atypical Rett syndrome raised the possibility of the CDKL5 gene mutations as a possible underlying genetic etiology for patients diagnosed with classical or atypical variants of Rett syndrome who presented with early seizures and were negative for the methyl-CpG-binding protein-2 (MECP2) gene mutation typically associated with Rett syndrome (Tao et al, 2004; Weaving et al, 2004; Mari et al, 2005; Scala et al, 2005; Bahi-Buisson et al, 2008a). This underlying genetic mutation would be the underpinning of a new clinical disease entity, later to be known as CDKL5 deficiency disorder.

The clinical characteristics commonly associated with a CDKL5 mutation include early-onset seizures, severe intellectual/gross motor impairment, and specific dysmorphic features. Epilepsy presents early in just about all patients afflicted with a CDKL5 gene deletion mutation. The typical seizures are either infantile spasms (i.e., West syndrome) or multifocal myoclonic seizures (Archer et al, 2006; Bahi-Buisson et al, 2008b; Mei et al, 2010). Early severe epileptic seizure disorder is accompanied by very limited developmental progress and marked hypotonia. Patients with CDKL5 gene abnormalities are reported to be normal in the first days of life to subsequently exhibit early signs of poor developmental skills, including poor sucking and poor eye contact, even before seizure onset. Reduced fetal movements have been reported retrospectively by expecting mothers (Archer et al, 2006). Subsequently, absent purposeful hand use, severe developmental delay, and absent language skills become apparent (Archer et al, 2006; Bahi-Buisson et al, 2008b; Elia et al, 2008; Nemos et al, 2009; Mei et al, 2010; Neul et al. 2010; Melani et al, 2011). About one third of patients will eventually be able to walk (Bahi-Buisson et al, 2008b). Males are at the more severe end of the phenotypic spectrum, with virtually no motor acquisitions (Van Esch et al, 2007; Sartori et al, 2009; Melani et al, 2011), while a rare female patient may attain some small level of independence with an attainment of better-than-expected language and motor milestones (Archer et al, 2006). Prior to the identification of the association between the CDKL5 gene and Rett syndrome, a great number of CDKL5 patients were classified as atypical Rett syndrome with early seizures (Hanefeld variant), as the severe hypotonia, impaired psychomotor development, and stereotypic hand movements noted are within the clinical manifestations of typical Rett syndrome (Artuso R et al, 2010; Stalpers X L et al 2012; Nemos C et al, 2009). However, unlike Rett syndrome, the CDKL5 epileptic encephalopathy patient does not typically regress in later years. Patients with CDKL5 epileptic encephalopathy manifest similar sleep and breathing symptoms as patients with Rett syndrome: disturbed sleep characterized by difficulty falling asleep, frequent awakenings, low sleep efficiency, decrease in rapid eye movement (REM) sleep, bruxism, daytime somnolence, and apneas (central or obstructive). While the disturbance of sleep is likely related to the underlying neurological disorder, gastric reflux, seizures, and AEDs are likely contributing to some degree (Hagebeuck et al, 2012; Mangatt et al, 2016). Gastrointestinal symptoms are quite common in CDKL5 epileptic encephalopathy patients, with about 90% reporting to have experienced constipation, gastroesophageal reflux, and/or air-swallowing. The odds of experiencing constipation and reflux increase with age, particularly after the age of 10 years. Dysmorphic features in CDKL5 epileptic encephalopathy are reported to be subtle, with the exception of the acquired microcephaly (slowing of head growth in relation to height and weight gains). The spectrum of features is similar overall in females and males. Frequently observed facial features include: a prominent and/or broad forehead; high hairline; relative mid-face hypoplasia; deep-set but ‘large’ appearing eyes, and infraorbital shadowing. There are no approved or licensed therapies in the United States for the treatment of patients with CDKL5 deficiency disorder.

The clinical characteristics commonly associated with a CDKL5 mutation include early-onset medication-refractory seizures, severe intellectual and gross motor impairment and severe sleep disturbances. The clinical manifestations of CDKL5 deficiency disorder for which ganaxolone may demonstrate some degree of therapeutic benefit are summarized as:

### Refractory Epilepsy

Epilepsy presents early in just about all patients afflicted with a CDKL5 gene deletion mutation. The typical seizures are either infantile spasms (i.e., West syndrome) or multifocal myoclonic seizures (Archer et al, 2006; Bahi-Buisson et al, 2008b; Mei et al, 2010). Some patients show a peculiar seizure pattern with “prolonged” generalized tonic-clonic events, lasting 2 to 4 minutes, consisting of a tonic-vibratory contraction, followed by a clonic phase with series of spasms, gradually translating into repetitive distal myoclonic jerks. It has also been noted that seizures are generally highly polymorphic and many different seizure types can occur in the same patient, evolving with time.

From a cohort of 86 patients (77 females, 9 males) derived from an international Rett syndrome patient registry and database (InterRett; Fehr et al, 2013) reported that seizures occurred in all except 1 female. Seizures occurred by 3 months of age in about 90% of patients, with a mean age of presentation in females of 7.3 weeks (range 0.3 to 34.8 weeks), and slightly earlier in males at 6.4 weeks (range 2.1 to 13 weeks). The overall control of seizures was poor, with 52 of 72 (72%) females and 8 of 9 (89%) males having daily seizures (Fehr et al. 2013).

Data obtained primarily from the International CDKL5 Deficiency Disorder Database (ICDD, where “CDD” stands for CDKL5 Deficiency Disorder) reported a similar lack of seizure control (Mangatt et al, 2016). Information on seizure frequency was available for 137/145 patients of the cohort survey. Ninety-five individuals (69.3%, 95/137) were experiencing seizures daily, with average frequency of daily seizures ranging from 1 to 21 seizures. Of those who provided information on the number of daily seizures (n=82), approximately one third were experiencing at least 5 seizures every day.

### Severe Development Delay

Early severe epileptic seizure disorder is accompanied by very limited developmental progress and marked hypotonia. Patients with CDKL5 gene abnormalities are reported to be normal in the first days of life but to subsequently exhibit early signs of poor developmental skills, including poor sucking and poor eye contact, even before seizure onset. Reduced fetal movements have been reported retrospectively by expecting mothers (Archer et al, 2006). Subsequently, absent purposeful hand use, severe developmental delay, and absent language skills become apparent (Archer et al, 2006; Bahi-Buisson et al, 2008b; Elia et al, 2008: Nemos et al, 2009, Mei et al, 2010; Neul et al. 2010; Melani et al, 2011). About one third of patients will eventually be able to walk (Bahi-Buisson et al, 2008b). Males are at the more severe end of the phenotypic spectrum, with virtually no motor acquisitions (Van Esch et al, 2007; Sartori et al, 2009; Melani et al, 2011), while a rare female patient may attain some small level of independence with an attainment of better-than-expected language and motor milestones (Archer et al, 2006). Most children have severely impaired social interaction and lack gaze fixation (Guerrini, R and Parrini, E, 2012).

### Disturbed Sleep

Nearly all patients with CDKL5 deficiency disorder manifest disturbed sleep characterized by difficulty falling asleep, frequent awakenings, low sleep efficiency, decrease in rapid eye movement (REM) sleep, bruxism, daytime somnolence, and apneas (central or obstructive). While the disturbance of sleep is likely related to the underlying neurological disorder, gastric reflux, seizures, and AEDs are likely contributing to some degree (Hagebeuck et al, 2012; Mangatt et al, 2016).

Night waking is the most persistently occurring sleep problem, experienced by more than half of the patients. Night waking is particularly worrisome and disruptive to parents, as it is often accompanied by inconsolable screaming or loud laughing spells (Bahi-Buisson et al 2008b, Mangatt et al 2016). In a study by Mori, et al, impacts of caring for a child with the CDKL5 Deficiency Disorder on parental wellbeing and family quality of life were evaluated. Data were sourced from the International CDKL5 Deficiency Disorder Database to which 192 families with a child with a pathogenic CDKL5 mutation had provided data by January 2016. Emotional wellbeing was considerably impaired in this caregiver population, and was particularly associated with increased severity of child sleep problems (Mori et al, 2017).

### Severely Impaired Gross Motor Function

The ICDD has collected data from parents and has been able to provide statistics with respect to gross motor function. The sample size is relatively small, and it is important to note that these are parent-led data. Based on a sample size of 116 children (102 females and 14 males) from 17 different countries, and ages ranging from 4 months to 29 years (median age 6 years) for females and 2 years to 22 years 8 months (median age 9 years 2 months) for males, gross motor function findings were:
Rolling over: approximately 84% of girls versus 35% of boys
Sitting independently: 55% of girls versus 23% of boys
Crawling: nearly 21% of the girls versus 10% of boys
Standing independently: almost 20% of girls
Walking independently: almost 18.8% of girls
Run independently: 8% of girls

Most boys needed maximal support to sit, stand, transition, and walk, but in this study, 3 boys learned to stand with support, 2 of whom also learned to walk with support. Because of the number of boys that are affected by CDKL5 Deficiency Disorder, the sample size was very small. However, in the last 2 years, the International Foundation for CDKL5 Research (IFCR) has become aware of boys that are mildly affected in comparison to most boys and there have been reports that some are able to walk, run, and climb.

### Reduced Life Expectancy is Likely

Due to the rarity of CDKL5 Deficiency Disorder, very little is known about long term prognosis and life expectancy. Most of those patients who have been identified are under 18 years of age and it is often difficult to identify older children and adults due to the frequent lack of complete infant and childhood developmental records and genetic testing in this older population. However, there are a few adults identified living with this disorder in their 20's, 30's, and even 40's. There are identical twins living in Europe that are believed to be in their 50's. However, it is important to note that like any condition that affects multiple organ systems as CDKL5 Deficiency Disorder does, there is a higher possibility of loss of life due to the epilepsy syndrome and other factors that contribute to severe respiratory infections and gastrointestinal problems/failure.

Information from various social media sources in which the CDKL5 UK patient advocacy group participates, indicates that a number of younger children have died in the past few years predominantly due to either respiratory failure due to pneumonia or complications associated with gastrointestinal problems. A number of children have died unexpectedly, most likely to due to Sudden Unexpected Death in Epilepsy (SUDEP). Patients with CDKL5 deficiency disorder are at increased risk for SUDEP due to frequent generalized tonic-clonic seizures.

According to the American Academy of Neurology (Practice Guideline Summary: Sudden Unexpected Death in Epilepsy Incidence Rates and Risk Factors April, 2017):
It is likely that generalized tonic-clonic seizure (GTCS) occurrence (versus no GTCS occurrence) increases SUDEP risk, based on moderate confidence in the evidence from 2 Class II studies.
It is highly likely that GTCS frequency is associated with an increased SUDEP risk (based on 2 Class II studies upgraded to high from moderate because of magnitude of the effect). SUDEP risk increases 3-fold at a GTCS frequency of >3/year, compared with a GTCS frequency of 1-2/year.
It is likely that having a seizure within the past year increases SUDEP risk (moderate confidence in the evidence based on 2 Class II studies), as does having a seizure in the previous 5 years (moderate confidence in the evidence based on 1 Class I study) compared with being seizure free.

There are an estimated 1200 patients who have at one time been identified as having one of the CDKL5 mutations. It is unknown how many of those patients have a pathological mutation and only about 400 patients are currently included in various registries around the world. It is likely that many patients in these registries are deceased based upon social media reports, and others that are not within the 2 to 17-year-old age range. Any study of patients with this disorder is severely hampered by the difficulty of enrolling a sufficient number of subjects to execute an adequately powered, randomized, controlled study in which seizure count reduction or proportion of subjects who respond (classically defined as at least 50% reduction from baseline seizure count) is the primary efficacy endpoint. These studies typically enroll 200 to 400 subjects, which would be essentially the entire population of eligible subjects worldwide.

In future studies, a primary endpoint that measures the overall treatment effect in this specific population such as CGI-I, with secondary endpoints that capture the most clinically meaningful endpoints, in addition to seizure frequency-related endpoints will be constructed.

### PCDH19

The PCDH19 gene encodes a protein, protocadherin 19, which is part of a family of molecules supporting the communication between cells in the central nervous system. As a result of mutation, protocadherin 19 may be malformed, reduced in its functions or not produced at all.

The abnormal expression of protocadherin 19 is associated with highly variable and refractory seizures, cognitive impairment and behavioral or social disorders with autistic traits.

PCDH19 female predominant pediatric epilepsy affects approximately 15,000-30,000 females in the United States. This genetic disorder is associated with seizures beginning in the early years of life, mostly focal clustered seizures that can last for weeks.

The mutation of the PCDH19 gene has been associated with low levels of allopregnanolone.

Protocadherin 19 (PCDH19)-related epilepsy is a serious epileptic syndrome characterised by early-onset cluster seizures, cognitive and sensory impairment of varying degrees, and psychiatric and behavioural disturbances (Depienne et al, 2012a). PCDH19-related epilepsy is characterised as a rare disorder by the National Institutes of Health Office of Rare Diseases Research (NIH-pcdh19-related-female-limited-epilepsy). This disorder is caused by a mutation of the PCDH19 gene, the gene that encodes for protocadherin 19 on the X chromosome (Dibbens et al, 2008; Depienne and LeGuern, 2012b; Depienne et al, 2009). The mechanism by which this mutation contributes to the development of epilepsy and intellectual impairment is poorly understood, however protocadherin 19 is a transmembrane protein of calcium-dependent cell-cell adhesion molecules that is strongly expressed in neural tissue (e.g., hippocampus, cerebral cortex, thalamus, amygdale), and which appears to be related to synaptic transmission and formation of synaptic connections during brain development) (Depienne et al, 2014). PCDH19-related epilepsy has an unusual X-linked mode of genetic transmission, with the condition predominantly limited to females (Depienne and LeGuern, 2012b).

Those affected by this gene mutation were found to have decreased endogenous allopregnanolone levels compared to age-matched controls.

The clinical features of PCDH19-related epilepsy have been well characterised (Depienne and LeGuern, 2012b; Higurashi et al, 2013). The hallmark characteristics of PCDH19-related epilepsy are clusters of brief seizures, which start in infancy or early childhood (range 4-60 months; average age of onset=12.9 months), and varying degrees of cognitive impairment (Depienne and LeGuern, 2012b; Higurashi et al, 2013; www.pcdh19info.org; Specchio et al, 2011). The onset of the first cluster of seizures usually coincides with a fever (i.e., febrile seizures) or immunization, and subsequent seizures may be febrile or afebrile, however fevers may worsen the seizures (Depienne and LeGuern, 2012b; Higurashi et al, 2013; Marini et al, 2010). Patients with PCDH19-FPE may experience individual seizures in addition to clusters and multiple seizure types. In some patients, seizures improve as patients reach puberty, possibly due to increased endogenous levels of progesterone and allopregnanolone.

The seizure clusters are characterized by brief seizures lasting 1-5 minutes, often preceded by fearful screaming (Depienne and LeGuern, 2012b; Higurashi et al, 2013; Marini et al, 2010). These clusters can occur more than 10 times a day over several days, with varying amounts of time between seizure clusters (Depienne and LeGuern, 2012b). Patients with PCDH19-related epilepsy may experience one or several types of seizures over the course of the disorder, with generalized tonic-clonic, tonic, clonic, and/or focal seizures seen most commonly. Absence seizures, atonic seizures, and myoclonus may also occur, albeit less frequently (Depienne and LeGuern, 2012b; Marini et al, 2010; Scheffer et al, 2008). Status epilepticus can occur early in the course of the disorder; moreover, seizures are often refractory to treatment, especially in infancy and childhood. Of note, seizure frequency and resistance to treatment tends to decrease over time, with some patients becoming seizure-free in adolescence or maintained on monotherapy in adulthood (Depienne et al, 2012a; Specchio et al, 2011; Scheffer et al, 2008; Camacho et al, 2012).

PCDH19-related epilepsy is usually, but not always, associated with cognitive impairment. It is estimated that up to 75% of patients with PCDH19-related epilepsy have cognitive deficits, ranging from borderline to severe (Depienne et al, 2009; www.pcdh19info.org; Specchio et al, 2011; Scheffer et al, 2008). Development of the child usually follows one of three courses: normal development with regression following seizures, normal development with no regression, and delays from birth that continue through adulthood (www.pcdh19info.org). Cognitive impairment does not appear to be related to frequency severity of seizures (Depienne et al, 2012a; Specchio et al, 2011).

PCDH19-related epilepsy may also be associated with a variety of psychiatric disorders most notably autism or autistic features (up to 60% of patients), attention deficit hyperactivity disorder (ADHD), behavioural disorders, obsessive-compulsive disorder or motor stereotypies, aggression, and anxiety. (Depienne et al, 2013; Marini et al, 2010; www.pcdh19info.org; Scheffer et al, 2008). In addition, other neurological abnormalities may be present including sleep disturbances, ictal apnea, motor deficits, hypotonia, language delay, sensory integration problems, and dysautonomia (www.pcdh19info.org; Smith et al, 2018).

Mutations of the PCDH19 gene were first identified in 2008 in seven large families with epilepsy and mental retardation limited to females (EFMR), and subsequently in individuals originally diagnosed with Dravet Syndrome (DS) who did not show the characteristic genetic mutations (SCN1A) associated with DS (Dibbens et al, 2008; Depienne LeGuern et al, 2012b). Although the disorder shares clinical features with other early-onset epileptic encephalopathies such as DS, it is a unique disorder with a distinct evolution of symptoms, and specific genetic mutations of the PCDH19 gene. Since the discovery PCDH19-related epilepsy, a significant number of patients with this disorder have been diagnosed and the mutation associated with PCDH19 has become the second most relevant gene in the epilepsy field (Depienne LeGuern et al, 2012b; Higurashi et al, 2013; Marini et al, 2010).

Prior to discovery of the role of PCDH19 in paediatric epilepsy, many patients were diagnosed with DS. There are also several differences in the two disorders. Males are over-represented in the DS population (2:1 ratio males to females); conversely, females with PCDH19 mutations are severely affected and males with the mutation are usually phenotypically normal with regard to seizures and cognition (Depienne et al, 2009). Additional differences in the clinical manifestations of the two disorders also exist including differences in types of seizures (e.g., less myoclonous and absence seizures in PCDH19-related epilepsy patients).

Also, PCDH19 patients exhibit older mean age of seizure onset, increased incidence of seizure clusters, and lack photosensitivity when compared to those with DS (Trivisano et al, 2016 and Steel 2017).

Protocadherin19 (PCDH19) is an adhesion molecule within the cadherin superfamily and highly expressed in the central nervous system (CNS), particularly the brain. The mechanism by which mutation of this gene contributes to the development of epilepsy and intellectual impairment is poorly understood, however protocadherin 19 is a transmembrane protein of calcium-dependent cell-cell adhesion molecules that is strongly expressed in neural tissue (e.g., hippocampus, cerebral cortex, thalamus, amygdale), and which appears to be related to synaptic transmission and formation of synaptic connections during brain development (Depienne et al, 2009). PCDH19-related epilepsy has an unusual X-linked mode of genetic transmission, with the phenotype predominantly limited to females and carrier males are generally unaffected (Depienne, LeGuern et al, 2012b). The role of this gene in paediatric epilepsies was only discovered in 2008 (Dibbens et al, 2008). A large systematic review and meta-analysis of 271 PCDH19-variant individuals that have been reported on in the literature was recently published and provides a comprehensive review of the disorder as well as typical phenotypic outcomes due to this mutation (Kolc et al, 2018).

The prevalence of PCDH19-related epilepsy is largely unknown due to the recent discovery of the gene and its contributions to early-onset childhood epilepsy. A top-down population-based approach estimates approximately 5,755 children with PCDH19-related epilepsy in the U.S. This number was derived from 470,000 children (<18 years old) living in the U.S. with active epilepsy (Zack and Kobau 2017) of which approximately 24.5% of those children are believed to have epilepsies with genetic aetiologies (unweighted average of Trump et al, 2016, Berg et al, 2017 and Lindy et al, 2018). Of the approximately 112,800 children living with genetic epilepsies in the U.S., approximately 5% are believed to be related to pathogenic PCDH19 gene mutations (unweighted average of Trump et al, 2016 and Lindy et al, 2018). Despite this methodological approach, the number of children formally diagnosed with PCDH19-related epilepsy is believed to be considerably less than the estimate above.

The PCDH19 Alliance, the leading patient advocacy organization based in the United States, estimates that the number of formally diagnosed individuals with PCDH19-related epilepsy throughout the world is approximately 1,000. It is hypothesised that many individuals are misdiagnosed due to limited awareness of PCDH19 or undiagnosed due to lack of genetic testing access or reimbursement.

### Clinical Manifestations of the PCDH19 Gene Mutations

There is a large phenotypic spectrum in those affected by mutations of the PCDH19 gene with no genotype-phenotype correlation established to date. PCDH19 is largely characterised by early onset (˜10 months of age) seizures typically occurring in clusters. Seizures are typically initiated by a febrile illness trigger. There appears to be an offset of seizures at an age period that correlates with puberty although this observation varies (van Harssel et al, 2013 and Scheffer et al, 2008). In addition to seizure burden, affected individuals with PCDH19 mutations also experience significant intellectual disability (Depienne et al, 2009 and Marini et al, 2010) and behavioural dysregulation (Depienne et al, 2011 and Dibbens et al, 2008). There is some phenotypic overlap with PCDH19-related epilepsy and Dravet Syndrome (DS) although there have been many reports describing the unique clinical manifestations of each genetic epilepsy. Prior to discovery of the PCDH19 gene many patients were diagnosed with DS. In fact, it is believed that ˜25% of SCN1A negative patients diagnosed with DS are likely PCDH19 positive (Jonghe 2011). This figure will likely change as awareness of PCDH19-related epilepsy increases.

### Refractory Epilepsy

Seizures are of significant clinical burden, particularly early in life, to those with PCDH19-related epilepsy. Seizure onset occurs at approximately 8-12 months of age (Marini et al, 2010; Smith et al, 2018). Both generalised and focal seizures have been reported in this condition (Smith et al, 2018; Marini et al, 2010; Specchio et al, 2011). Absence seizures, atonic seizures, and myoclonus may also occur, albeit less frequently (Depienne and LeGuern 2012b; Marini et al, 2010; Scheffer et al, 2008). A hallmark characteristic of PCDH19 seizures are that they typically occur in clusters and are characterized by brief seizures lasting 1-5 minutes, often preceded by fearful screaming (Depienne and LeGuern 2012b; Higurashi et al, 2013; Marini et al, 2010). These clusters can occur more than 10 times a day over several days, with varying amounts of time between seizure clusters (Depienne and LeGuern 2012b). Patients with PCDH19-related epilepsy may experience one or several types of seizures over the course of the disorder. Status epilepticus can occur early in the course of the disorder; moreover, seizures are often refractory to treatment, especially in infancy and childhood.

### Intellectual Disability

Prior to the PCDH19 gene discovery, young girls with this condition were diagnosed as “epilepsy in females with mental retardation” (EFMR). Autism spectrum disorder (ASD) and intellectual disability (ID) are exhibited in 75-80% in individuals with PCDH19 mutations (Breuillard et al, 2016; Smith et al, 2018). Cognitive outcomes are very heterogeneous with a range of mild to severe impairment. ID has been diagnosed by low scores in all cognitive domains but with more significant impairment in theory of mind. There has been no correlation between the severity of epilepsy and level of ID (Specchio et al, 2011; Depienne et al, 2011).

### Behavioural Dysregulation

Behavioral and psychiatric comorbidities are well-described in affected individuals with PCDH19 gene mutation. These problems include aggressiveness, depressed mod, and psychotic traits. A large meta-analysis of 271 individuals with PCDH19 variants reported that 60% of females, 80% of affected mosaic males, and nine hemizygous males developed psychiatric characteristics commonly including hyperactivity, autistic features, and obsessive-compulsive behaviours (Kolc et al, 2018). Further, it is common for behavioral and psychiatric disorders to be a primary area of patient and caregiver concern. Whereas seizure burden typically decreases with age, behavioural and psychiatric comorbidities remain relatively unchanged throughout life.

### Disturbed Sleep

Sleep dysregulation has also been reported as a common attribute of PCDH19-related epilepsy and one of significant concern for families. These disturbances have been described as trouble falling and/or staying asleep. Sleep disturbances were reported in 53% (20/38) probands mainly described as sleep maintenance insomnia with many children waking up too early and struggling to return to sleep (Smith et al, 2018). It is unknown how seizure activity may correlate with sleep dysfunction and vice versa.

### The PCDH19 Gene and Protein

The PCDH19 gene is located on the long (q) arm of the X chromosome at position 22.1 and its coding sequence consists of six exons. This gene encodes a 1148 amino acid protein, protocadherin 19, which is a member of the protocadherin family and plays a critical role in cell-cell interactions. Protocadherins, including PCDH19, play an important role in axon guidance/sorting, neurite self-avoidance, and synaptogenesis (Garret and Weiner 2009; Lefebvre et al, 2012).

The majority of PCDH19-related epilepsy gene mutations were observed in the extracellular domain of the protein encoded by exon 1. Missense variants are most common (˜45%), following by frameshift (27%), and nonsense (20%) mutations (Kolc et al, 2018).

PCDH19-related epilepsy is an X-linked disorder in which, paradoxically, females with point mutations of the PCDH19 gene are severely impacted, whereas transmitting males are not. Usually, in most X-linked dominant disorders, males are more severely affected than females, and often die in utero. In a large series of cases in which inheritance was determined, half of the PCDH19 mutations occurred de novo, and half were inherited from fathers who were healthy, and who had no evidence of seizures or cognitive disorders (Depienne et al, 2012a; Depienne et al, 2009). The expression of PCDH19 mutations is highly variable, with some individuals being hardly affected, and others showing severe disease. Even monozygotic twins with the mutation may have variations in seizure frequency and degree of cognitive impairment (Higurashi et al, 2013).

There are several hypothesized mechanisms for this unusual mode of transmission including the presence of a compensatory protocadherin gene on the Y chromosome or cellular interference (Depienne et al, 2012a; Depienne et al, 2009). With regard to the latter, in case of mutation, two cellular populations may arise, one with mutated PCDH19 and one with the normal gene. This natural mosaic may be harmful to normal brain functioning. Males, since they have only one X chromosome and one copy of the PCDH19 gene, will have a single uniform cellular population in the event of mutation, which does not appear to harm brain cells. The fact that non-mosaic hemizygous males do show the phenotype of PCDH19-related epilepsy suggests that PCDH19 protein may be non-essential in humans.

### Unmet Therapeutic Need

There remains a clear and significant unmet medical need for individuals affected by PCDH19-related epilepsy. To date there are no approved drugs or therapies indicated for this specific patient population. Individuals are currently being treated with various anti-epileptic drugs (AEDs) without any established standard of care. Further, some of the anti-seizure medication has significant negative side-effects and exacerbates other outcomes such as behaviour. Therefore, there is a need for a safe, durable drug that can effectively control seizures while also potentially assisting with the other neuropsychiatric disorders.

### Need for Improved Seizure Control

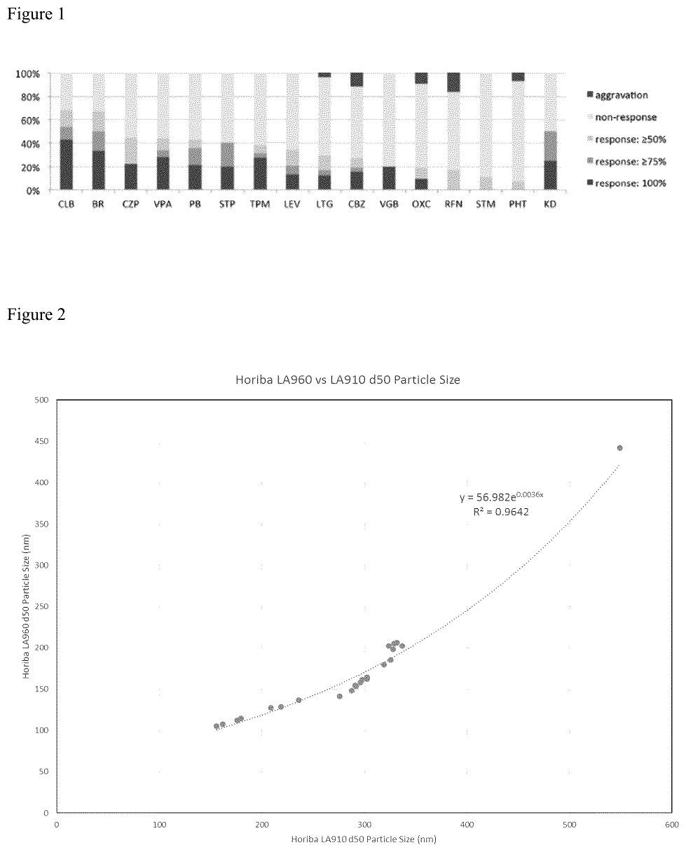

Despite the availability of many AEDs, their therapeutic efficacy is limited and highly variable in this patient population. Lotte et al retrospectively reviewed the efficacy of AED's in 58 females with PCDH19-related epilepsy. The findings are depicted in FIG. 1. Despite reported moderate efficacy with clobazam, many individuals continue to experience seizures and remain not adequately treated.

In addition, multiple other reports have described the majority of PCDH19-related epilepsy patients experience uncontrolled refractory seizures. Fifty-eight (58%) of the probands in a cohort of 38 individuals remained refractory to 3 or more seizure medications (Smith et al, 2018). Further, recent research only described 17 probands with ‘controlled’ seizures out of 271 probands (Kolc et al, 2018).

Currently there are no anti-epileptic drugs (AEDs) approved for PCDH19-related epilepsy so there remains a significant unmet need in this patient population.

During the first several years of PCDH19-related epilepsy, seizure clusters are frequent and severe, and may persist despite appropriate treatment ultimately becoming treatment-refractory (Higurashi et al, 2013). Despite the many available AEDs, there are none currently available that provide consistent control of seizures in PCDH19-related epilepsy patients. Higurashi and associates explored the efficacy of AEDs in patients with PCDH19-related epilepsy (Higurashi et al, 2013). The authors noted that carbamazepine had very low efficacy, especially in children that experience strong cluster seizures. After dose reduction or discontinuation of midazolam (which can control seizures in these patients) seizure recurrence and sometimes seizure aggravation have been observed. Other AEDs, such as phenytoin/fosphenytoin or phenobarbital showed only transient efficacy. In addition, Smith et al. reported on a cohort of 38 patients with PCDH19-related epilepsy captured in a patient registry. Of these patients, 30 (79%) still demonstrate uncontrolled seizures despite many of them being on greater than or equal to 3 AEDs (Smith et al, 2018). For these reasons, there is a need for new AEDs with novel mechanisms of action and improved side effect profiles that can maintain seizure control for people with PCDH19-related epilepsy.

Thus, there is unmet medical need for PCDH19-related epilepsy, a distinct generic epilepsy. The formulations and methods disclosed herein may fulfil this need.

In addition to the methods disclosed herein, there is a potential for ganaxolone to also have positive effects on the neuropsychiatric, behavioural, and sleep disorders associated with PCDH19-related epilepsy. A potential drug treatment that can provide multi-modal action related to the various symptoms these individuals face would be a therapeutic improvement to current standard of care. Such a treatment would be within the scope of the present invention.

### Reduced Steroidogenesis in Patients with PCDH19-Related Epilepsy

Endogenous neurosteroids play a critical role in maintaining homeostasis of brain activity. Two recent reports have provided compelling evidence that endogenous neurosteroid productive is decreased in those affected by PCDH19 gene mutation.

Tan et al. were the first to report this phenomenon. They performed gene expression analysis on primary skin fibroblasts of those affected by PCDH19-related epilepsy as well as age-matched controls. They reported that the AKR1C1-3 genes were significantly dysregulated when compared to controls. These genes are known to be critical in producing steroid hormone-metabolizing enzymes responsible for generating allopregnanolone. This gene expression result was further confirmed by analytical assessment of allopregnanolone in the blood (Tan et al, 2015).

The finding of Tan et al. was further supported when Trivisano et al, reported on blood levels of various neurosteroids, including allopregnanolone, in 12 PCDH19 patients and compared the levels to age-matched controls. In general they found reduced steroidogenesis in those affected by the gene mutation (Trivisano et al, 2017).

Therefore, administration of the pregnenolone neurosteroid may help to minimize the effects of allopregnanolone deficiency.

### Dravet Syndrome

Dravet syndrome is a rare genetic epileptic encephalopathy described in 1978. It begins in the first year of life in an otherwise healthy infant. Prior to 1989, this syndrome was known as epilepsy with polymorphic seizures, polymorphic epilepsy in infancy (PMEI) or severe myoclonic epilepsy in infancy (SMEI). The disease begins in infancy but is lifelong.

About 80% of people with this syndrome have a gene mutation (SCN1A is the most frequent) that causes problems in the way that ion channels in the brain work. Approximately 95% of patients with Dravet syndrome have de novo heterozygous mutations, which explains the unaffected status of many siblings and parents.

The first seizure is often associated with a fever and may be a tonic clonic seizure or a seizure involving clonic movements on 1 side of the body. The seizures are refractory in most cases. Most children develop some level of developmental disability and have other conditions that are associated with the syndrome. Infants have normal development at the time the seizures begin, magnetic resonance imaging (MRI) and electroencephalogram (EEG) tests are also normal in infancy.

Seizures early in life are often prolonged (lasting more than 2 minutes) or repetitive and can result in status epilepticus. Children with Dravet syndrome can develop many different seizure types: myoclonic seizures, tonic clonic seizures, absence or atypical absence seizures, atonic seizures, partial seizures, non-convulsive status epilepticus. Myoclonic seizures appear between 1 and 5 years in 85% of children with Dravet syndrome.

Seizures occur without a fever. However, these children are very sensitive to infections and frequently have seizures when they are ill or have a fever. Seizures can also be triggered by slight changes in body temperature that are not caused by infection for example a warm or hot bath water or hot weather. Many children have photosensitive seizures. Emotional stress or excitement can also trigger seizures in some children.

Children usually develop normally in the early years. After age 2, they may lose developmental milestones or do not progress as quickly as they get older and have more seizures. There seems to be a correlation between frequency of seizures, how often status epilepticus occurs, and the degree of developmental delay in children. Around 6 years of age, cognitive problems in some children may stabilize or may start improving. However, most children with Dravet syndrome have some degree of developmental disability that persists.

Other problems that may be seen include: low motor tone—can lead to painful foot problems, unsteady walking, some may develop a crouched gait, chronic infections, low humoral immunity, growth and nutrition problems, problems with the autonomic nervous system and behavioral or developmental problems such as autism spectrum disorder.

### LGS

Lennox-Gastaut syndrome (LGS) is a severe form of epilepsy. Seizures usually begin before 4 years of age. Seizure types, which vary among patients, include tonic, atonic, atypical absence, and myoclonic. There may be periods of frequent seizures mixed with brief, relatively seizure-free periods.

Most children with LGS experience some degree of impaired intellectual functioning or information processing, along with developmental delays, and behavioral disturbances. Lennox-Gastaut syndrome can be caused by brain malformations, perinatal asphyxia, severe head injury, central nervous system infection and inherited degenerative or metabolic conditions. In 30 to 35 percent of cases, no cause can be found. Many cases of LGS have had genetic mutations associated with the diagnosis clinically. These can include known encephalopathic epilepsy genes in Rett Syndrome, CNTNAP1, XP22.33, SCN2A, GABR3, Shank2, Shank3, and other genetic conditions associated with LGS-type clinical epilepsy.

Patients with LSG and other genetically based conditions with intractable epilepsy clinically resembling LGS conditions have been, at times, treated with and responsive to classes of corticosteroids like prednisone or adrenocorticotropic hormone (ACTH).

Non-degenerative genetic types of LGS or idiopathic refractive cases may respond to the neurosteroid treatment as described herein.

### CSWS

Continuous spike wave in sleep (CSWS) starts with seizures between 2 to 12 years; peaks at 4 to 5 years with EEG continuous spikes and waves during slow-wave sleep, usually 1 to 2 years from seizure onset22. Males (62%) show preponderance and up to ⅓ of the patients have abnormal mental state. The clinical manifestations include 3 stages of evolution:

First stage before CSWS: infrequent nocturnal motor focal seizures, often hemiclonic status epilepticus, absences, atonic, complex focal seizures, and generalized tonic-clonic seizures occur.

Second stage with CSWS: seizures more frequent and complicated with typical or more frequent atypical absences, myoclonic absences, absence status epilepticus, rarely atonic or clonic seizures, and focal simple or partial complex dyscognitive seizures, usually nocturnally during CSWS condition on EEG and some secondary or primary generalized tonic-clonic seizures. Tonic seizures do not occur. Eminent psychomotor decline and behavioral abnormalities, and a Wernicke's type or global language regression occurs with localization of perisylvian cortex on EEG and magnetoencephalography (MEG) studies.

Third stage (after months to usually 2 to 10 years) with remission of CSWS and seizures and general improvement, normalization of CSWS pattern, and residual language or other learning difficulties.

New genetic overlap to autism genetics and epilepsy genetics have been noted, mainly Grin2A or Grin2B among others. Many may be idiopathic to testing.

### Early Infantile Epileptic Encephalopathy

Early infantile epileptic encephalopathy is a genetic disease that affects newborns. It is characterized by seizures. Infants have primarily tonic seizures (which cause stiffening of muscles of the body, generally those in the back, legs, and arms), but may also experience partial seizures, and rarely, myoclonic seizures (which cause jerks or twitches of the upper body, arms, or legs). Episodes may occur more than a hundred times per day.

### Status Epilepticus (SE)

Status epilepticus (SE) is a serious seizure disorder in which the epileptic patient experiences a seizure lasting more than five minutes, or more than one seizure in a five minute period without recovering between seizures. In certain instances convulsive seizures may last days or even weeks. Status epilepticus is treated in the emergency room with conventional anticonvulsants. GABA_{A} receptor modulators such as benzodiazepines (BZs) are a first line treatment. Patients who fail to respond to BZs alone are usually treated with anesthetics or barbiturates in combination with BZs. About 23-43% of status epilepticus patients who are treated with a benzodiazepine and at least one additional antiepileptic drug fail to respond to treatment and are considered refractory (Rossetti, A. O. and Lowenstein, D. H., Lancet Neurol. (2011) 10(10): 922-930.) There are currently no good treatment options for these patients. The mortality rate for refractory status epilepticus (RSE) patients is high and most RSE patients do not return to their pre-RSE clinical condition. About 15% of patients admitted to hospital with SE are in a subgroup of RSE patients said to be super-refractory SE (SRSE), in which the patients have continued or recurrent seizures 24 hours or more after the onset of anesthetic therapy. SRSE is associated with high rates of mortality and morbidity. (Shorvon, S., and Ferlisi, M., Brain, (2011) 134(10): 2802-2818.)

### Early Severe Epileptic Seizure

Early severe epileptic seizure disorder is accompanied by very limited developmental progress and marked hypotonia.

### Fragile X Syndrome (FXS)

Fragile X is a genetic condition that is characterized by range of developmental problems including learning disabilities and cognitive impairment.

### Neurosteroid

Endogenous neurosteroids play a critical role in maintaining homeostasis of brain activity. Neurosteroids have the ability to enact brain changes rapidly in response to changes in the brain environment. Neurosteroids are devoid of interactions with classical steroid hormone receptors that regulate gene transcription; they modulate brain excitability primarily by interaction with neuronal membrane receptors and ion channels.

Neurosteroids can be positive or negative regulators of the GABA_{A} receptor function, depending on the chemical structure of the steroid molecule (Pinna and Rasmussen, 2014, Reddy, 2003). The GABA_{A} receptor mediates the lion's share of synaptic inhibition in the CNS. Structurally, GABA_{A} receptors are hetero-pentamers of 5 protein subunits to form the chloride ion channels. There are 7 different classes of subunits, some of which have multiple homologous variants (α1-6, β1-3, γ1-3, σ1-3, δ, ε, θ); most GABA_{A} receptors are composed of α, θ and γ or δ subunits. The neurotransmitter GABA activates the opening of chloride ion channels, permitting chloride ion influx and ensuing hyperpolarisation. GABA_{A} receptors prevent action potential generation by swerving the depolarisation produced by excitatory neurotransmission. There are 2 types of inhibitory neurotransmission mediated via GABA_{A} receptors: synaptic (phasic) and extrasynaptic (tonic) inhibition. Neurosteroids modulate both synaptic and extrasynaptic GABA_{A} receptors, and thereby potentiate both phasic and tonic currents. Phasic inhibition results from the activation of γ2-containing receptors at the synapse by intermittent release of millimolar concentrations of GABA from presynaptic GABA-ergic inter-neurons' axon terminals. Tonic inhibition, in contrast, is mediated by the continuous activation of δ-containing extra-synaptic receptors outside of the synaptic cleft by low levels of ambient GABA which escaped reuptake by GABA transporters. Tonic inhibition plays a unique role in controlling hippocampus excitability by setting a baseline of excitability (Reddy 2010).

Neurosteroids such as ganaxolone are potent positive allosteric modulators of GABA_{A} receptors (Akk et al, 2009). The first observation that neurosteroids enhance GABA-evoked responses that are mediated by GABA_{A} receptors was reported in 1984 with alphaxolone (Harrison and Simmonds, 1984). This modulating effect of neurosteroids occurs by binding to discrete sites on the GABA_{A} receptor that are located within the transmembrane domains of the α- and β-subunits (Hosier et al, 2007; Hosier et al, 2009). The binding sites for neurosteroids are distinct from that of the GABA, benzodiazepine, and barbiturate. Although the exact locations of neurosteroid binding sites are currently unknown, it has been shown that a highly conserved glutamine at position 241 in the M1 domain of the α-subunit plays a key role in neurosteroid modulation (Hosie et al, 2009). In addition to the binding sites, there are also differences between neurosteroids and benzodiazepines in their respective interactions with GABA_{A} receptors. While neurosteroids modulate most GABA_{A} receptor isoforms, benzodiazepines only act on GABA_{A} receptors that contain γ2-subunits and do not contain α4- or α6-subunits (Lambert et al, 2003; Reddy, 2010). The specific α-subunit may influence neurosteroid efficacy, whereas the γ-subunit type may affect both the efficacy and potency for neurosteroid modulation of GABA_{A} receptors (Lambert et al, 2003).

Recent studies have indicated the existence of at least 3 neurosteroid binding sites on the GABA_{A} receptor: 1 for allosteric enhancement of GABA-evoked currents by allopregnanolone, 1 for direct activation by allopregnanolone, and 1 for antagonist action of sulfated neurosteroids such as pregnanolone sulfate, at low (nM) concentrations (Lambert et al, 2003; Hosie et al, 2007). Neurosteroid enhancement of GABA_{A} receptor chloride currents occurs through increases in both the channel open frequency and channel open duration (Reddy, 2010). Thus, neurosteroids greatly enhance the probability of GABA_{A} receptor chloride channel opening that allows a massive chloride ion influx, thereby promoting augmentation of inhibitory GABA-ergic transmission. These effects occur at physiological concentrations of neurosteroids. Thus, endogenous neurosteroid levels continuously modulate the function of GABA_{A} receptors (Reddy, 2010).

The extra-synaptic δ-subunit containing GABA_{A} receptors exhibit increased sensitivity to neurosteroids, suggesting a key modulatory role in tonic inhibition (Wohlfarth et al., 2002). GABA_{A} receptors that contain the δ subunit are more sensitive to neurosteroid-induced potentiation of GABA responses (Stell et al, 2003). Mice lacking δ subunit show drastically reduced sensitivity to neurosteroids (Mihalek et al, 1999). The δ-subunit does not contribute to the neurosteroid binding site, but appears to confer enhanced transduction of neurosteroid action after the neurosteroid has bound to the receptor. GABA_{A} receptors containing the δ-subunit have a low degree of desensitisation, facilitating the mediating tonic GABA_{A} receptor currents that are activated by ambient concentrations of GABA in the extracellular space. Tonic GABA_{A} receptor current causes a steady inhibition of neurons and reduces their excitability. GABA is a relatively low efficacy agonist of δ-containing GABA_{A} receptors even though it binds with high affinity (Glykys and Mody, 2007). Thus, neurosteroids can markedly enhance the current generated by δ-containing GABA_{A} receptors even in the presence of saturating GABA concentrations. During neuronal activity, there is expected to be substantial release of GABA from active GABA-ergic interneurons that can interact with perisynaptic and extrasynaptic δ-subunit containing GABA_{A} receptors. Overall, the robust effect of neurosteroids is likely to be due to their action on both synaptic and perisynaptic/extrasynaptic GABA_{A} receptors (Reddy, 2010).

Pregnane neurosteroids and pregnenolone neurosteroid are a class of compounds useful as anesthetics, sedatives, hypnotics, anxiolytics, anti-depressants, anti-tremor, a treatment for autistic behavior, and anticonvulsants. These compounds are marked by very low aqueous solubility, which limits their formulation options. The present invention provides nanoparticulate formulations of pregnane and pregnenolone neurosteroids that are bioavailable orally and parenterally.

Injectable formulations of pregnane neurosteroids and pregnenolone neurosteroid are particularly desirable as these compounds are used for clinical indications for which oral administration is precluded, such as anesthesia and particularly for the emergency treatment of active seizures.

The disclosure includes injectable nanoparticle neurosteroid formulations.

The pregnane neurosteroid and pregnenolone neurosteroid of the present invention may each be a compound of Formula IA:

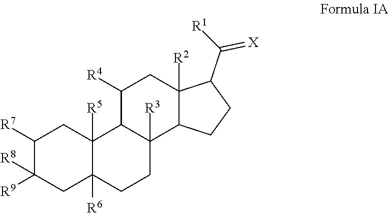

or a pharmaceutically acceptable salt thereof, wherein:
X is O, S, or NR¹⁰;
R¹ is hydrogen, hydroxyl, —CH₂A, optionally substituted alkyl, optionally substituted heteroalkyl, optionally substituted aryl, or optionally substituted arylalkyl;
A is hydroxyl, O, S, NR¹¹, or optionally substituted nitrogen-containing five-membered heteroaryl or optionally substituted nitrogen-containing bicyclic heteroaryl or bicyclic heterocyclyl,
R⁴ is hydrogen, hydroxyl, oxo, optionally substituted alkyl, or optionally substituted heteroalkyl,
R², R³, R⁵, R⁶, and R⁷ are each independently absent, hydrogen, hydroxyl, halogen, optionally substituted a C₁-C₆ alkyl, optionally substituted a C₁-C₆alkoxyl (e.g., methoxyl) or optionally substituted heteroalkyl;
R⁸ and R⁹ are each independently selected from a group consisting of hydrogen, a C₁-C₆ alkyl (e.g., methyl), a halogenated C₁-C₆ alkyl (e.g., trifluoromethyl) or C₁-C₆alkoxyl (e.g., methoxyl), or R⁸ and R⁹ form an oxo group;
R¹⁰ is hydrogen, hydroxyl, optionally substituted alkyl, optionally substituted heteroalkyl, optionally substituted aryl, or optionally substituted arylalkyl where each alkyl is a C₁-C₁₀alkyl, C₃-C₆cycloalkyl, (C₃-C₆cycloalkyl)C₁-C₄alkyl, and optionally contains a single bond replaced by a double or triple bond;
each heteroalkyl group is an alkyl group in which one or more methyl group is replaced by an independently chosen —O—, —S—, —N(R¹⁰)—, —S(═O)— or —S(═O)₂—, where R¹⁰ is hydrogen, alkyl, or alkyl in which one or more methylene group is replaced by —O—, —S—, —NH, or —N-alkyl;
R¹¹ is —H₂ or —HR¹²;
R¹² is C₁-C₆ alkyl or C₁-C₆ alkoxy.

The pregnane neurosteroid and pregnenolone neurosteroid of the present invention may each be a compound of Formula IA, wherein
X is O;
R¹ is hydrogen, —CH₃, —CH₂OH, 1H-imidazol-1-yl, 1-oxidoquinolin-6-yloxyl and 4-cyano-1H-pyrazol-1′-yl.
R⁴ is hydrogen, hydroxyl, oxo, optionally substituted alkyl, or optionally substituted heteroalkyl,
R², R³, R⁵, R⁶, and R⁷ are each independently absent, hydrogen, hydroxyl, halogen, optionally substituted a C₁-C₆ alkyl, optionally substituted a C₁-C₆alkoxyl (e.g., methoxyl) or optionally substituted heteroalkyl;
R⁸ and R⁹ are each independently selected from a group consisting of hydrogen, a C₁-C₆ alkyl (e.g., methyl), a halogenated C₁-C₆ alkyl (e.g., trifluoromethyl) or C₁-C₆alkoxyl (e.g., methoxyl), or R⁸ and R⁹ form an oxo group;
R¹⁰ is hydrogen, hydroxyl, optionally substituted alkyl, optionally substituted heteroalkyl, optionally substituted aryl, or optionally substituted arylalkyl where each alkyl is a C₁-C₁₀alkyl, C₃-C₆cycloalkyl, (C₃-C₆cycloalkyl)C₁-C₄alkyl, and optionally contains a single bond replaced by a double or triple bond;
   each heteroalkyl group is an alkyl group in which one or more methyl group is replaced by an independently chosen —O—, —S—, —N(R¹⁰)—, —S(═O)— or —S(═O)₂—, where R¹⁰ is hydrogen, alkyl, or alkyl in which one or more methylene group is replaced by —O—, —S—, —NH, or —N-alkyl.

The pregnane neurosteroid and pregnenolone neurosteroid of the present invention may each be a compound of Formula IB

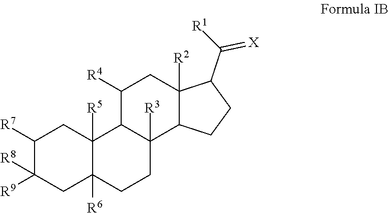

or a pharmaceutically acceptable salt thereof, wherein:
X is O, S, or NR¹⁰;
R¹ is hydrogen, hydroxyl, optionally substituted alkyl, optionally substituted heteroalkyl, optionally substituted aryl, or optionally substituted arylalkyl;
R⁴ is hydrogen, hydroxyl, oxo, optionally substituted alkyl, or optionally substituted heteroalkyl,
R², R³, R⁵, R⁶, and R⁷ are each independently hydrogen, hydroxyl, halogen, optionally substituted alkyl, or optionally substituted heteroalkyl;
R⁸ is hydrogen or alkyl and R⁹ is hydroxyl; or
R⁸ and R⁹ are taken together to form an oxo group;
R¹⁰ is hydrogen, hydroxyl, optionally substituted alkyl, optionally substituted heteroalkyl, optionally substituted aryl, or optionally substituted arylalkyl where each alkyl is a C₁-C₁₀alkyl, C₃-C₆cycloalkyl, (C₃-C₆cycloalkyl)C₁-C₄alkyl, and optionally contains a single bond replaced by a double or triple bond;
   each heteroalkyl group is an alkyl group in which one or more methyl group is replaced by an independently chosen —O—, —S—, —N(R¹⁰)—, —S(═O)— or —S(═O)₂—, where R¹⁰ is hydrogen, alkyl, or alkyl in which one or more methylene group is replaced by —O—, —S—, —NH, or —N-alkyl.

Compounds of Formula IA and IB include, e.g., allopregnanolone, ganaxolone, alphaxalone, alphadolone, hydroxydione, minaxolone, pregnanolone, acebrochol, or tetrahydrocorticosterone, and pharmaceutically acceptable salts thereof.

The pregnane neurosteroid and pregnenolone neurosteroid of the present invention may also each be a compound of Formula II:

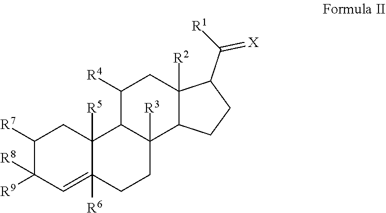

or a pharmaceutically acceptable salt thereof, wherein:
X is O, S, or NR¹⁰;
R¹ is hydrogen, hydroxyl, —CH₂A, optionally substituted alkyl, optionally substituted heteroalkyl, optionally substituted aryl, or optionally substituted arylalkyl;
A is hydroxyl, O, S, NR¹¹ or optionally substituted nitrogen-containing bicyclic heteroaryl or bicyclic heterocyclyl,
R⁴ is hydrogen, hydroxyl, oxo, optionally substituted alkyl, or optionally substituted heteroalkyl,
R², R³, R⁵, R⁶, and R⁷ are each independently absent, hydrogen, hydroxyl, halogen, optionally substituted a C₁-C₆ alkyl, optionally substituted a C₁-C₆alkoxyl (e.g., methoxyl) or optionally substituted heteroalkyl;
R⁸ and R⁹ are each independently selected from a group consisting of hydrogen, a C₁-C₆ alkyl (e.g., methyl), a halogenated C₁-C₆ alkyl (e.g., trifluoromethyl) or C₁-C₆alkoxyl (e.g., methoxyl), or R⁸ and R⁹ form an oxo group;
R¹⁰ is hydrogen, hydroxyl, optionally substituted alkyl, optionally substituted heteroalkyl, optionally substituted aryl, or optionally substituted arylalkyl where each alkyl is a C₁-C₁₀alkyl, C₃-C₆cycloalkyl, (C₃-C₆cycloalkyl)C₁-C₄alkyl, and optionally contains a single bond replaced by a double or triple bond;
each heteroalkyl group is an alkyl group in which one or more methyl group is replaced by an independently chosen —O—, —S—, —N(R¹⁰)—, —S(═O)— or —S(═O)₂—, where R¹⁰ is hydrogen, alkyl, or alkyl in which one or more methylene group is replaced by —O—, —S—, —NH, or —N-alkyl;
R¹¹ is —H₂ or —HR¹²; R¹² is C₁-C₆ alkyl or C₁-C₆ alkoxy.

The pregnane neurosteroid and pregnenolone neurosteroid of the present invention may also each be a compound of Formula III:

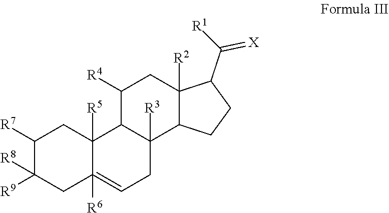

or a pharmaceutically acceptable salt thereof, wherein:
X is O, S, or NR¹⁰;
R¹ is hydrogen, hydroxyl, —CH₂A, optionally substituted alkyl, optionally substituted heteroalkyl, optionally substituted aryl, or optionally substituted arylalkyl;
A is hydroxyl, O, S, NR¹¹ or optionally substituted nitrogen-containing bicyclic heteroaryl or bicyclic heterocyclyl,
R⁴ is hydrogen, hydroxyl, oxo, optionally substituted alkyl, or optionally substituted heteroalkyl,
R², R³, R⁵, R⁶, and R⁷ are each independently absent, hydrogen, hydroxyl, halogen, optionally substituted a C₁-C₆ alkyl, optionally substituted a C₁-C₆alkoxyl (e.g., methoxyl) or optionally substituted heteroalkyl;
R⁸ and R⁹ are each independently selected from a group consisting of hydrogen, a C₁-C₆ alkyl (e.g., methyl), a halogenated C₁-C₆ alkyl (e.g., trifluoromethyl) or C₁-C₆alkoxyl (e.g., methoxyl), or R⁸ and R⁹ form an oxo group;
R¹⁰ is hydrogen, hydroxyl, optionally substituted alkyl, optionally substituted heteroalkyl, optionally substituted aryl, or optionally substituted arylalkyl where each alkyl is a C₁-C₁₀alkyl, C₃-C₆cycloalkyl, (C₃-C₆cycloalkyl)C₁-C₄alkyl, and optionally contains a single bond replaced by a double or triple bond;
each heteroalkyl group is an alkyl group in which one or more methyl group is replaced by an independently chosen —O—, —S—, —N(R¹⁰)—, —S(═O)— or —S(═O)₂—, where R¹⁰ is hydrogen, alkyl, or alkyl in which one or more methylene group is replaced by —O—, —S—, —NH, or —N-alkyl;
R¹¹ is —H₂ or —HR¹²
R¹² is C₁-C₆ alkyl or C₁-C₆ alkoxy.

### Ganaxolone

Ganaxolone (CAS Reg. No. 38398-32-2, 3α-hydroxy-3β-methyl-5α-pregnan-20-one) (GNX) is a new chemical entity under investigation as an antiepileptic drug (AED) for use in rare pediatric seizure disorders, e.g., protocadherin (PCDH)19 female predominant epilepsy, also known as PCDH19 female-limited epilepsy, epilepsy associated with cyclin-dependent kinase-like 5 (CDKL5) mutation (DCKL5 deficiency disorder), and Lennox-Gastaut syndrome, with additional potential utility in Dravet Syndrome, Angelman Syndrome, status epilepticus and neuropsychiatric disorders and behaviors such as fragile X syndrome (FXS), postpartum depression, premenstrual dysphoric disorder, and other mood or movement disorders.

The structural formula of ganaxolone is:

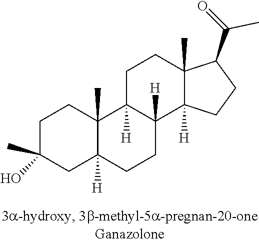

Ganaxolone is the 3β-methylated synthetic analog of the endogenous neurosteroid allopregnanolone, an endogenous allosteric modulator of γ-aminobutyric acid type A (GABA_{A}) receptors in the central nervous system (CNS). Ganaxolone has the same core chemical structure as allopregnanolone, but with the addition of a 33 methyl group designed to prevent conversion back to an entity that is active at nuclear hormone receptors, thereby eliminating the opportunity for unwanted hormonal effects while enhancing the bioavailability of the neurosteroid and preserving its desired CNS activity.

Like allopregnanolone, ganaxolone, (a neuroactive steroid), exhibits potent antiepileptic, anxiolytic, sedative and hypnotic activities in animals by allosterically modulating γ-aminobutyric acid type A (GABA_{A}) receptors in the central nervous system (CNS). Ganaxolone has potency and efficacy comparable to allopregnanolone in activating synaptic and extrasynaptic GABA_{A} receptors at a site distinct from the benzodiazepine site.

Ganaxolone works by interacting with both synaptic and extrasynaptic GABA_{A} receptors at binding sites which are unique to the class. Outside of the synapse, ganaxolone can be absorbed into the cell membrane and diffuse to activate the extrasynaptic GABA_{A} receptors, providing constant, or tonal, modulation of the GABA inhibitory signal that calms overexcited neurons.

Ganaxolone has anti-convulsant activity and is useful, e.g., in treating epilepsy and other central nervous system disorders.

Ganaxolone is insoluble in water. Its solubilities in 95% alcohol, propylene glycol and polyethylene glycol are 13 mg/mL, 3.5 mg/mL and 3.1 mg/mL, respectively.

Ganaxolone is primarily metabolized by the CYP3A family of liver enzymes, but interactions based on hepatic metabolism are limited to those caused by induction or inhibition of CYP3A4/5 by other drugs such as ketoconazole.

In vitro, the clearance of ganaxolone appears to be driven mainly by CYP3A4. In clinical studies in adults, administration of grapefruit increased the exposure of ganaxolone in healthy volunteers. Levels of ganaxolone were reduced in patients treated concomitantly with enzyme-inducing AEDs. These data further support the hypothesis of CYP3A4 being a major contributor to the clearance of ganaxolone in humans.

In adults, plasma concentrations of ganaxolone after oral administration are characterized by high variability. Single-dose PK parameters were strongly influenced by the rate and extent of ganaxolone absorption, and whether the subjects were in the fed or fasted state.

In the pediatric population, the level of CYP3A4 expression approaches that of adults by approximately 2 years of age (de Wildt et al, 2003), albeit with a high-degree of inter-individual variability. Therefore, patients greater than 2 years of age would be expected to have ganaxolone clearance rates similar to adults.

Ganaxolone has a relatively long half-life—approximately 20 hours in human plasma following oral administration (Nohria, V. and Giller, E., Neurotherapeutics, (2007) 4(1): 102-105). Furthermore, ganaxolone has a short Tₘₐₓ, which means that therapeutic blood levels are reached quickly. Thus initial bolus doses (loading doses) may not be required, which represents an advantage over other treatments. Ganaxolone is useful for treating seizures in adult and pediatric epileptic patients.

Ganaxolone affects GABA_{A} receptors by interacting with a recognition site that is distinct from other allosteric GABA_{A} receptor modulators such as benzodiazepines. Ganaxolone binds to intra- and extrasynaptic receptors, mediating both phasic and tonic modulation, respectively. The unique binding of Ganaxolone to these 2 receptors does not lead to the tolerance seen with benzodiazepines. In contrast to allopregnanolone, ganaxolone is orally bioavailable and cannot be back-converted in the body to intermediates such as progesterone, with classical steroid hormone activity, and as such, does not directly or indirectly via metabolic conversion activate the progesterone receptor.

Ganaxolone administered intravenously was also evaluated and shown to induce burst suppression-like electroencephalogram (EEG) patterns in otherwise normal rats and block seizure response in models that represent clinical status epilepticus (SE). Ganaxolone caused a sedative response but did not cause a full anaesthetic response.

In addition to anticonvulsant activity, ganaxolone has been shown to have anxiolytic properties as well as improve behaviours associated with autism. In a mouse model of posttraumatic stress disorder (PTSD), Ganaxolone treatment decreased aggression and social isolation-induced anxiety-like behaviour (Pinna and Rasmussen, 2014). In another study, ganaxolone treatment improved sociability in the BTBR mouse model of autism (Kazdoba et al, 2016). A clinical study of ganaxolone treatment of children and adolescents with fragile X syndrome (FXS), ganaxolone reduced anxiety and hyperactivity and improved attention in those with higher baseline anxiety (Ligsay et al, 2017).

Ganaxalone has been shown to exhibit potent antiseizure activity in numerous animal models and has been shown to be safe and effective in preliminary studies in children with refractory epilepsy (Nohria and Giller, 2007).

The anticonvulsant activity of ganaxalone was established in multiple in vivo models of seizure activity. The results from these studies show that ganaxalone blocks seizure propagation, elevates seizure threshold and can reverse status epilepticus with acute or delayed administration.

Safety pharmacology studies were conducted with ganaxalone.

Ganaxalone did not interact with the human ether-a-go-go related gene (hERG) receptor at a measured concentration of 70 nM (n=2). Ganaxalone had no effect on cardiovascular parameters in dogs following a single dose of up to 15 mg/kg (maximum concentration [Cmax] of 1000 ng/mL and area under the concentration time curve (AUCXO)(0-24) of 10000 ng·h/mL). In the 1-year dog toxicity study (Cmax>1500 ng/mL), transient sinus tachycardia (>190 beats per minute [bpm]) was observed after 3 months of dosing in 4 animals and was accompanied by decreased PR and QT interval but no treatment effect on QRS duration or Q-T interval corrected (QTc). No pulmonary effects were observed in female rats at doses up to 40 mg/kg.

There was a physiologically normal shortening of the PR and QT interval in response to the higher heart rate. There was no effect on QRS duration or QTc interval. No pulmonary effects were observed in female rats at doses up to 40 mg/kg.

Ganaxalone induces major cytochrome P450 (CYP) isoenzymes 1A1/2 and 2B1/2 in female rats but not males. Auto-induction has also been observed in the mouse and rat while no auto-induction has been observed in dogs.

Tissue distribution studies in mice and rats have demonstrated that [¹⁴C]-ganaxolone was rapidly distributed throughout the body into highly perfused organs, intestine, and adipose tissue, with brain ganaxolone concentrations approximately 5-fold higher than those in plasma.

Most excreted radioactivity in all species is via faeces (>70%) with the remaining excreted in urine.

The most common effect following treatment with ganaxalone in toxicology studies was dose-related sedation, an expected pharmacological effect of a positive modulator of GABA_{A} receptors. In both the oral and IV programmes, there was little evidence of target organ or systemic toxicity associated with either single- or multiple-dose treatment with ganaxalone. No functional or anatomic changes within haematopoietic tissue or any specific organ such as liver, kidney or gastrointestinal (GI) systems were seen in the repeat-dose studies. In rats, ganaxalone induced hepatic enzymes, with more pronounced effects in females, which were correlated to increased liver weights and dose related hepatocellular hypertrophy in a 6-month study.

In the chronic oral toxicity study in dogs, mean Cₘₐₓ levels of greater than 1500 ng/mL (10 and 15 mg/kg/day) were associated with increased weight and total plasma cholesterol levels.

When given IV to rats and dogs, the main dose limiting toxicity finding was sedation. The no observed adverse effect level (NOAEL) after IV dosing in rats for 14 days was established at 42 mg/kg/day for males and 30 mg/kg/day for females. The NOAEL in dog after administration of ganaxolone by IV bolus followed by continuous IV infusion for 28 days was 7.20 mg/kg/day, which corresponded to a steady-state concentration of approximately 330 ng/mL and 333 ng/mL. There were no findings in a local tolerance study in rabbits. Finally, in vitro ganaxalone did not cause haemolysis and was compatible with human plasma.

Ganaxalone was not teratogenic in rats or mice and did not significantly affect the development of offspring. ganaxalone had no effects on fertility and early embryonic development in rats. No potential for mutagenicity was detected. Treatment of neonatal rats with ganaxalone produced expected signs of sedation but did not affect development or demonstrate any post-mortem changes.

In the oral dosing programme, a therapeutic index from non-human NOAEL levels to the adult partial-onset seizure epilepsy and the pharmacokinetics study is approximately 2 to 3-fold in dogs (sedation).

Ganaxolone has been shown to stop generalized convulsive seizures in both animal models of epilepsy and status epilepticus.

In addition to reducing seizures, ganaxolone may benefit behavioral comorbidities as well as sleep in subjects with genetic epilepsies.

In one aspect of the present invention, ganaxolone is used in the treatment of rare pediatric seizure disorders such as protocadherin (PCDH)19-pediatric epilepsy, also known as PCDH19-related epilepsy, cyclin-dependent kinase-like 5 (CDKL5) deficiency disorder (CDD), and Lennox-Gastaut Syndrome (LGS), with additional potential utility in status epilepticus (SE) and neuropsychiatric disorders and behaviours such as fragile X syndrome (FXS), postpartum depression, premenstrual dysphoric disorder, and other mood disorders.

### Allopregnalone

Allopregnanolone (CAS Reg. No. 516-54-1, 3α,5α-tetrahydroprogesterone) is an endogenous progesterone derivative with anti-convulsant activity.

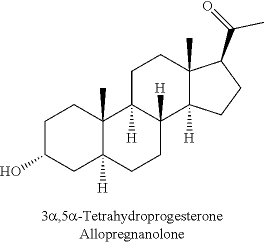

Allopregnanolone has a relatively short half-life, about 45 minutes in human plasma.

Allopregnanolone exhibits potent antiepileptic, anxiolytic, sedative and hypnotic activities in animals by virtue of its GABA_{A} receptor modulating activity.

In addition to its efficacy in treating seizures, allopregnanolone is being evaluated for use in treating neurodegenerative diseases including Alzheimer's disease, Parkinson's disease, Huntington's disease, and amyotrophic lateral sclerosis and for treating lysosomal storage disorders characterized by abnormalities in cholesterol synthesis, such as Niemann Pick A, B, and C, Gaucher disease, and Tay Sachs disease. (See U.S. Pat. No. 8,604,011, which is hereby incorporated by reference for its teachings regarding the use of allopregnanolone for treating neurological disorders.)

It has been hypothesized that disturbances in certain neurosteroid hormones, such as allopregnanolone, may be implicated in the molecular pathogenesis of PCDH19-related epilepsy (Tan et al, 2015 and Trivisano et al, 2017). Allopregnanolone is a neurosteroid that has known anticonvulsant and anxiolytic effects acting as a positive allosteric modulator of the GABA_{A} receptor. Gecz and colleagues have studied various aspects of PCDH19-related epilepsy molecular pathology (Tan et al, 2015). Expression analysis of PCDH19-related epilepsy skin fibroblasts suggests downregulation of certain sex-based genes in this disorder. The AKR1C genes are those that are most consistently altered. When skin cell preparations from girls with PCDH19 mutations and controls were stimulated with progesterone, the fibroblasts from the PCDH19-mutation patients were poorer metabolisers of progesterone into allopregnanolone. This suggests that compromised AKR1C mRNA, protein levels, and enzymatic activity may lead to allopregnanolone deficiency in patients with PCDH19-related epilepsy. Gecz and colleagues are currently studying additional preclinical models to assess allopregnanolone deficiency in PCDH19-related epilepsy (Tan et al, 2015).

The relationship between progesterone and its metabolite, allopregnanolone, and seizures has been extensively studied in women with catamenial epilepsy, a condition in which there are changes in seizure frequency associated with different phases of the menstrual cycle. At times during the menstrual cycle when progesterone is lower (e.g., perimenopause), the likelihood of seizures tends to increase (French 2005). Circulating allopregnanolone levels parallel those of progesterone. While the reproductive effects of progesterone are related to its interaction with intracellular progesterone receptors, the anticonvulsant effects of progesterone are not (Reddy and Rogawski 2009). The antiseizure activity of progesterone results from its conversion to the neurosteroid, allopregnanolone (Kokate et al, 1999). Allopregnanolone has been shown to protect against seizure activity in a number of animal models, due to its effects on GABA_{A} receptors (Reddy and Rogawski 2009). Ganaxolone, a synthetic analog of allopregnanolone devoid of progesterone-related effects, may be useful in the treatment of seizures associated with PCDH19-related epilepsy.

### Alphaxalone

Alphaxalone, also known as alfaxalone, (CAS Reg. No. 23930-19-0, 3α-hydroxy-5α-pregnan-11, 20-dione) is a neurosteroid with an anesthetic activity. It is used as a general anaesthetic in veterinary practice. Anaesthetics are frequently administered in combination with anti-convulsants for the treatment of refractory seizures. An injectable nanoparticle neurosteroid dosage form containing alphaxalone alone or in combination with either ganaxolone or allopregnanolone is within the scope of this disclosure.

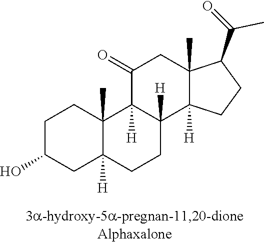

### Aphadolone

Alphadolone, also known as alfadolone, (CAS Reg. No. 14107-37-0, 3α, 21-dihydroxy-5α-pregnan-11, 20-dione) is a neurosteroid with anaesthetic properties. Its salt, alfadolone acetate is used as a veterinary anaesthetic in combination with alphaxalone.

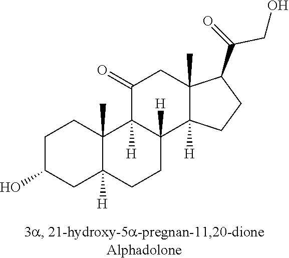

### Additional Neurosteroids

Newly published data provide further evidence that pregnenolone, a neurosteroid related to ganaxolone, may specifically aid in repair of the neuronal damage caused by CDKL5 deficiency disorder. The kinase CDKL5, which is deficient in patients with CDKL5 gene mutations, is required for IQ motif containing GTPase activating protein 1 (IQGAP1) to form a functional complex with its effectors, Rac1, and the microtubule plus end tracking protein, CLIP170. This complex is needed for targeted cell migration and polarity, both of which impact neuronal morphology. CDKL5 deficiency disorder disrupts the microtubule association of CLIP170, thus deranging their dynamics. CLIP170 is a cellular target of pregnenolone, a neurosteroid that is very similar in structure and function to ganaxolone. By blocking CLIP170 in its active conformation, pregnenolone can restore the microtubule association of CLIP170 in CDKL5 deficient cells and rescuing morphological defects in neurons devoid of CDKL5 (Barbiero I, Peroni D, Tramarin M, Chandola C, Rusconi L, Landsberger N, Kilstrup-Nielsen C. The neurosteroid pregnenolone reverts microtubule derangement induced by the loss of a functional CDKL5-IQGAP1 complex. Hum Mol Genet. 2017 Jun. 21. doi:10.1093/hmg/ddx237. [Epub ahead of print]). These findings provide novel insights into CDKL5 function and pave the way for target-specific therapeutic strategies such as ganaxolone for individuals affected with CDKL5-disorder.

Additional neurosteroids that may be used in the nanoparticle neurosteroid formulation of this disclosure and the methods disclosed herein include hydroxydione (CAS Reg. No. 303-01-5, (5β)-21-hydroxypregnane-3,20-dione), minaxolone (CAS Reg. No. 62571-87-3, 2β,3α,5α,11α)-11-(dimethylamino)-2-ethoxy-3-hydroxypregnan-20-one), pregnanolone (CAS Reg. No. 128-20-1, (3α,5β)-d-hydroxypreganan-20-one), renanolone (CAS Reg. No. 565-99-1, 3α-hydroxy-5β-pregnan-11,20-dione), or tetrahiydrocorticosterone (CAS Reg. No. 68-42-8, 3α, 5α-pregnan-20-dione).

Additional neurosteroids that may be used in the nanoparticle neurosteroid formulation of this disclosure and the methods disclosed herein include Co26749/WAY-141839, Co134444, Co177843, and Sage-217, Sage-324 and Sage-718. Co26749/WAY-141839, Co134444, Co177843, and Sage-217 have the following structures:

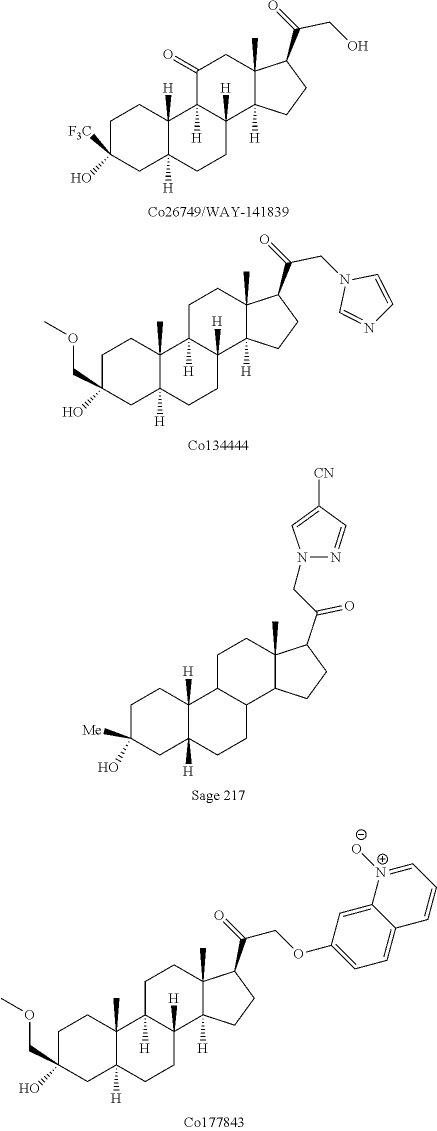

Additional neurosteroids that may be used in the nanoparticle neurosteroid formulation of this disclosure and the methods disclosed herein include compounds disclosed in U.S. Patent Publication No. 2016-0229887 (U.S. Ser. No. 14/913,920, filed Feb. 23, 2016), herein incorporated by reference in its entirety.

### Dosage

The pregnenolone neurosteroid in the methods of present invention can be administered in the amount of from about 1 mg/day to about 5000 mg/day in one, two, three, or four divided doses. In certain embodiments, doses of 1600 mg/day and 2000 mg/day maybe associated with somnolence, and a dose of 1800 mg/day defines the optimal combination of drug exposure, dosing convenience and tolerability.

When the pregnenolone neurosteroid is ganaxolone, a target and maximum dose of ganaxolone is about 1800 mg/day. In these embodiments, this dose provides the highest feasible exposure based on the non-linear kinetics of ganaxolone. Thus, when the pregnenolone neurosteroid is ganaxolone, the amount of ganaxolone administered in the methods of the invention is generally from about 200 mg/day to about 1800 mg/day, from about 300 mg/day to about 1800 mg/day, from about 400 mg/day to about 1800 mg/day, from about 450 mg/day to about 1800 mg/day, from about 675 mg/day to about 1800 mg/day, from about 900 mg/day to about 1800 mg/day, from about 1125 mg/day to about 1800 mg/day, from about 1350 mg/day to about 1800 mg/day, from about 1575 mg/day to about 1800 mg/day, or about 1800 mg/day, at a dose of from 1 mg/kg/day to about 63 mg/kg/day in one, two, three or four divided doses.

In certain embodiments, from about 900 mg to about 1800 mg, from about 950 mg to about 1800 mg, from about 1000 mg to about 1800 mg, from about 1100 mg to about 1800 mg, or from about 1200 mg of ganaxolone is administered per day, for two or more consecutive days. Ganaxolone may be administered orally or parenterally in one, two, three, or four doses, per day.

Whether a human receives ganaxolone twice or three times daily depends on the formulation. For patients dosing with oral immediate release capsules, ganaxolone is generally administered twice a day, each dose separated from the subsequent and/or previous dose by 8 to 12 hours. For patients taking oral suspension, ganaxolone is generally administered three times a day, each dose separated from the subsequent and/or previous dose by 4 to 8 hours.

When the pregnenolone neurosteroid is ganaxolone, the methods of the invention comprise administration of ganaxolone at a dose of from 1 mg/kg/day to about 63 mg/kg/day, provided that the total amount of administered ganaxolone does not exceed 1800 mg/day.

The pharmacokinetics of ganaxolone in formulations comprising immediate release 0.3-micron particles (e.g., the formulation of Example 2) are linear through approximately 1200 mg/day (given twice-a-day (“BID”)), with a modest increase in exposure at a dose of 1600 mg/day, and little or no further increase in exposure at a dose of 2000 mg/day. Therefore, to maintain as high a trough level as possible in all subjects, a dose of 1800 mg is generally targeted. A dose level higher than 1800 mg/day would not be medically advantageous because it would not lead to greater exposure and furthermore would require more than three times daily dosing which may hamper patient compliance.

In certain embodiments, ganaxolone is administered at a dose of more than 5 mg/kg/day, for example a dose of from about 6 mg/kg/day to about 63 mg/kg/day, provided that the total amount of administered ganaxolone does not exceed 1800 mg/day.

In certain embodiments, the dose of ganaxolone is adjusted in 15 mg/kg/day up to 63 mg/kg/day up to the maximum dose of 1800 mg per day during treatment.

In certain embodiments, the method of treatment comprises administering at least 33 mg/kg/day of ganaxolone in one, two, three, or four doses, with a maximum daily dose of about 1800 mg.

In certain embodiments, the human is from about of 0.6 and about 7 years old and is administered a dose of ganaxolone of from about 1.5 mg/kg BID (3 mg/kg/day) to 12 mg/kg (three times a day (“TID”) (36 mg/kg/day). In the embodiments, where the human receives 12 mg/kg TID dose regimen, and the trough concentrations of at least about 38.5±37.4 ng/mL is achieved.

In certain embodiments, ganaxolone is administered orally to 5-15 year old humans at doses of 6 mg/kg BID (12 mg/kg/day) to 12 mg/kg TID (36 mg/kg/day) in a β-cyclodextrin formulation with food, and ganaxolone's plasma concentrations of up to 22.1 ng/mL and 5.7 to 43.7 ng/mL are achieved at week 4 and week 8, respectively, of the administration.

In certain embodiments, ganaxolone is given orally in the same formulation to 1 to 13 year old epilepsy patients with food at doses of 1 to 12 mg/kg TID (3 to 36 mg/kg/day), and ganaxolone plasma concentrations of up to 5.78 ng/mL (1 mg/kg TID) to 10.3 to 16.1 ng/mL (12 mg/kg TID) are achieved.

In certain embodiments, ganaxolone is given orally to patients aged 4 to 41 months (0.33 to 3.42 years) at a dose of 3 to 18 mg/kg TID (9 to 54 mg/kg/day) in an oral suspension formulation, and ganaxolone Cₘₐₓ of about 123 ng/mL and a trough concentration of about 23 ng/mL is achieved.

In certain embodiments, mean ganaxolone Cₘᵢₙ. (trough) are from 55 ng/ml to about 100 ng/ml, and Cₘₐₓ levels are from about 240 ng/ml to 400 ng/ml (e.g., 262 ng/mL), based on three-times-a-day administration of 1000 mg ganaxolone in the 0.3 μm ganaxolone suspension (i.e., formulation of Example 1).

In certain embodiments, the methods result in mean Cₘᵢₙ (trough) and Cₘₐₓ levels are about 56.9 ng/ml and about 262 ng/mL, respectively, based on twice-a-day administration of 1000 mg ganaxolone in the 0.3 micron ganaxolone capsule formulation (i.e., formulation of Example 2).

In certain embodiments, administration of ganaxolone provides a Cₘᵢₙ/Cₘₐₓ ratio of greater than 3, 3.5, 4, 4.5, 5, or 6. This Cₘᵢₙ/Cₘₐₓ ratio may be provided after a single dose administration and/or after administration at steady-state. In certain embodiments, the Cₘᵢₙ/Cₘₐₓ ratio remains the same, regardless of the dose of ganaxolone administered.

In certain embodiments, the dose administered is determined from a pediatric pharmacokinetic model that allows a determination of the dose of ganaxolone in the various pediatric age ranges that will produce a Cₘₐₓ and AUC exposure similar to that achieved following an efficacious dose determined in the adult epilepsy population. The model could, e.g., be constructed with standard methods with consideration of the pharmacokinetic data in the present application.

In certain embodiments, the pregnenolone neurosteroid may be administered to the patient using a number of titration steps until a therapeutically effective dosage regimen is attained. For example, about six-eight titration steps may be used, depending on the size of the patient.

In certain embodiments, the method of treatment of the invention comprises establishing a baseline seizure frequency for the patient, initially administering a dose of ganaxolone to the patient in an amount from about 0.5 mg/kg/day to about 15 mg/kg/day; and progressively increasing the dose of ganaxolone over the course of 4 weeks to an amount from about 18 mg/kg/day to about 60 mg/kg/day, wherein the total dose of ganaxolone is up to about 1800 mg/day for patients whose body weight is greater than 30 kg. For patients whose body weight is 30 kg or less, the total dose of ganaxolone per day may be less (e.g., about 63 mg/day). In certain preferred embodiments, the initial dose of ganaxolone is about 4.5 mg/kg/day. In certain preferred embodiments, the ganaxolone dose is increased to about 36 mg/kg/day. In certain preferred embodiments, the ganaxolone dose is decreased to a prior level if the patient experiences dose-limiting adverse events.

In certain embodiments, for subjects weighing more than 30 kg, treatment is initiated at a dose of 900 mg/day in divided doses. The dose is then increased by approximately 20 to 50% (e.g., an increase from 900 mg/day to 1200 mg/day is a 33% increase) at intervals of not less than 3 days and not more than 2 weeks, provided that the current dose is reasonably tolerated, until desired efficacy is achieved or a maximally tolerated dose (MTD) level is reached. Subsequent dose adjustments may be made in increments of approximately 20 to 50% with a minimum of 3 days between dose changes, unless required for safety. The maximum allowable dose in these embodiments is 1800 mg/day.

In certain embodiments, for subjects weighing 30 kg, or less, treatment is initiated at 18 mg/kg/day and may be increased in about 20% to 50% increments at intervals of not less than 3 days and not more than 2 weeks, provided that the current dose is reasonably tolerated, until desired efficacy is achieved or a maximally tolerated dose (MTD) level is reached. Subsequent dose adjustments may be made in increments of ˜20% to 50% with a minimum of 3 days between dose changes, unless required for safety. The maximum allowable dose these embodiments is 63 mg/kg/day.

For humans weighing ≥28 kg (62 lbs), ganaxolone may be initiated at a dose of from about 300 mg/day to about 600 mg/day (e.g., 400 mg/day) in divided doses. The dose will be increased 450 mg/day every 7 days until 1800 mg/day is reached or a maximum tolerated dose.

For humans weighing <28 kg (62 lbs), ganaxolone may be initiated at a dose of from about 10 mg/kg/day to about 30 mg/kg/day (e.g., 18 mg/kg/day), increasing approximately 15 mg/kg/day every week until 63 mg/kg/day is reached.

In certain embodiments, ganaxolone is administered in increments of from 10 mg/day to 20 mg/day (e.g., 15 mg/kg/day) up to 63 mg/kg/day (maximum 1800 mg/day) as an oral suspension or in increments of from 225 mg/day to 900 mg/day (e.g., 450 mg/day) as an oral capsule. In some of these embodiments, ganaxolone may, e.g., be dosed as follows:
6 mg/kg three times daily (TID) (18 mg/kg/day) suspension/225 twice daily (BID) (450 mg/day) capsules-Days 1-7;
11 mg/kg TID (33 mg/kg/day) suspension/450 BID (900 mg/day) capsules—Days 8-14;
16 mg/kg TID (48 mg/kg/day) suspension/675 BID (1350 mg/day) capsules—Days 15-21;
21 mg/kg TID (63 mg/kg/day not to exceed 1800 mg/day) suspension/900 BID (1800 mg/day) capsules—Days 22-28.

In certain embodiments, ganaxolone is administered in oral suspension, and the following titration schedule is used:

| | | | | | | |
|---|---|---|---|---|---|---|
| | | | Dose | Total | % | Total |
| Titration | | Dose | mg/kg | mg | Dose | ml |
| Step # | mg/kg | (mg) | increase | change | Change | Suspension |

15 kg (33 lbs)

| | | | | | | |
|---|---|---|---|---|---|---|
| 1 | 18 | 270 | | | | 5.4 |
| 2 | 24 | 359 | 6 | 89 | 33% | 7.2 |
| 3 | 32 | 478 | 8 | 119 | 33% | 9.6 |
| 4 | 42 | 635 | 11 | 158 | 31% | 12.7 |
| 5 | 54 | 810 | 12 | 175 | 27% | 16.2 |
| 6 | 63 | 945 | 9 | 135 | 16% | 18.9 |

20 kg (44 lbs)

| | | | | | | |
|---|---|---|---|---|---|---|
| 1 | 18 | 360 | | | | 7.2 |
| 2 | 24 | 479 | 6 | 119 | 33% | 9.6 |
| 3 | 32 | 637 | 8 | 158 | 33% | 12.7 |
| 4 | 42 | 847 | 10 | 210 | 31% | 16.9 |
| 5 | 54 | 1080 | 12 | 233 | 27% | 21.6 |
| 6 | 63 | 1260 | 9 | 180 | 16% | 25.2 |

25 kg (55 lbs)

| | | | | | | |
|---|---|---|---|---|---|---|
| 1 | 18 | 450 | | | | 9.0 |
| 2 | 24 | 599 | 6 | 149 | 33% | 12.0 |
| 3 | 32 | 796 | 8 | 198 | 33% | 15.9 |
| 4 | 42 | 1059 | 10 | 263 | 31% | 21.2 |
| 5 | 54 | 1350 | 12 | 291 | 27% | 27.0 |
| 6 | 63 | 1575 | 9 | 225 | 16% | 31.5 |

30 kg (66 lbs)

| | | | | | | |
|---|---|---|---|---|---|---|
| 1 | 18 | 540 | | | | 10.8 |
| 2 | 24 | 718 | 6 | 178 | 33% | 14.4 |
| 3 | 32 | 955 | 8 | 237 | 33% | 19.1 |
| 4 | 42 | 1270 | 10 | 315 | 31% | 25.4 |
| 5 | 50 | 1500 | 8 | 230 | 18% | 30.0 |
| 6 | 55 | 1650 | 5 | 150 | 11% | 33.0 |
| 7 | 60 | 1800 | 5 | 150 | 9% | 36.0 |

| | 200 mg capsules | | | 225 mg capsules | | |
|---|---|---|---|---|---|---|
| | Total | No. | No. | Total | No. | No. |
| Titration | Daily | Caps | Caps | Daily | Caps | Caps |
| Step | Dose | AM | PM | Dose | AM | PM |
| 1 | 400 | 1 | 1 | 450 | 1 | 1 |
| 2 | 600 | 1 | 2 | 675 | 1 | 2 |
| 3 | 800 | 2 | 2 | 900 | 2 | 2 |
| 4 | 1000 | 2 | 3 | 1125 | 2 | 3 |
| 5 | 1200 | 3 | 3 | 1350 | 3 | 3 |
| 6 | 1400 | 3 | 4 | 1575 | 3 | 4 |
| 7 | 1600 | 4 | 4 | 1800 | 4 | 4 |
| 8 | 1800 | 4 | 5 | | | |

In certain embodiments, the trough concentrations associated with maximal efficacy are in the range of about 55 ng/mL, about 60 ng/ml or about 65 ng/ml (0.3 micron suspension; TID dosing) and a dose of 1800 mg/day (0.3 micron capsules, BID dosing) provides trough plasma concentrations in this range.

Methods of treatment disclosed herein encompass administration of neurosteroid (e.g., ganaxolone) with or without food. In certain embodiments, ganaxolone is administered with food.

### Treatment Duration

Treatment duration in accordance with the present invention may range from 1 day to more than 2 years. For example, treatment duration may be from 1 day to 80 years, from 1 day to 70 years, from 1 day to 60 years, from 1 day to 50 years, from 1 day to 45 years, from 2 days to 45 years, from 2 days to 40 years, from 5 days to 35 years, from 10 days to 30 years, from 10 day to 30 years, from 15 days to 30 years. In some embodiments, the treatment duration is for as long as the subject continues to derive a therapeutic benefit from administration of the neurosteroid. In some embodiments, the treatment duration is 14 days, 28 days, 30 days, 6 weeks, 8 weeks, 10 weeks, 12 weeks, 6 months, 1 year, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, or 10 years.

In certain embodiments, at the conclusion of the treatment period, or upon discontinuation of the treatment, the dose is gradually decreased over a period of 1 to 4 weeks, based on subject's age, weight, dose and duration of the treatment.

### Formulations

The formulations of the present invention comprise a pregnenolone neurosteroid (e.g., ganaxolone) and one or more pharmaceutically acceptable excipient(s). In certain embodiments, the formulations are free from cyclodextrins, including sulfoalkyl ether cyclodextrins and modified forms thereof.

In the preferred embodiments, the amount of the pregnenolone neurosteroid in the formulation is therapeutically effective to treat a symptom of a disorder selected from the group comprising or consisting from PCDH19-related epilepsy, CDKL5 epileptic encephalopathy, Dravet Syndrome, Lennox-Gastaut syndrome (LGS), Continuous Sleep Wave in Sleep (CSWS), Epileptic Status Epilepticus in Sleep (ESES), and other intractable and refractory genetic epilepsy conditions that share common seizure types and clinically resemble PCDH19-related epilepsy, CDKL5 Deficiency Disorder, Dravet Syndrome, LGS, CSWS, and ESES upon administration of the formulation for 1 week and/or 2 weeks and/or 3 weeks and/or 4 weeks and/or 6 weeks and/or 7 weeks, and/or 8 weeks, and/or 9 weeks and/or 10 weeks and/or 11 weeks and/or 12 weeks. The symptom may be selected from the group consisting of refractory epilepsy, developmental delay, intellectual disability, disturbed sleep, impaired gross motor function, behavioral dysregulation, and combinations of two or more of the foregoing. In some of these embodiments, the amount of the pregnenolone neurosteroid is effective to reduce seizure frequency in a human after administration at a dosage and duration described in the present specification.

In preferred embodiments of the present invention, the pregnenolone neurosteroids such as ganaxolone are incorporated into a pharmaceutically acceptable composition for oral administration. Such a formulation in certain preferred embodiments may be a liquid (e.g., an aqueous liquid (encompassing suspensions, solutions and the like). In other preferred embodiments, the oral formulation may be an oral solid dosage form (e.g., an oral capsule or tablet). In most preferred embodiments, the oral formulation is an oral suspension comprising the pregnenolone neurosteroid. Preferably, a unit dose of the oral formulation contains a therapeutically effective amount of the pregnenolone neurosteroid which can be orally administered to the (e.g., human) patient (e.g., an infant, child, adolescent or adult). In certain embodiments, the oral suspension is administered to the patient via the use of an oral syringe. For example, it is contemplated that the oral suspension is utilized for children who weigh less than 30 kg. On the other hand, the oral suspension may be administered to those patients who would have trouble swallowing a solid oral dosage form. Children larger than 30 kg may take a solid dosage form, e.g., ganaxolone capsules. The ganaxolone oral suspension may be administered through an oral dosing syringe, e.g., three times daily. The ganaxolone capsules may be administered, e.g., twice daily. The patients experience better absorption of the ganaxolone with meals (milk).

In certain preferred embodiments, the liquid formulation of the present invention may be a formulation as described and prepared in Applicant's prior U.S. Pat. No. 8,022,054, entitled “Liquid Ganaxolone Formulations and Methods for the Making and Use Thereof”, hereby incorporated by reference in its entirety. However, the oral liquid (e.g., suspension) formulation of pregnenolone neurosteroid may be prepared in accordance with other methods known to those skilled in the art.

As described in U.S. Pat. No. 8,022,054, the liquid formulation may be an aqueous dispersion of stabilized pregnenolone neurosteroid (e.g., ganaxolone) particles comprising ganaxolone, a hydrophilic polymer, a wetting agent, and an effective amount of a complexing agent that stabilizes particle growth after an initial particle growth and endpoint is reached, the complexing agent selected from the group of small organic molecules having a molecular weight less than 550 and containing a moiety selected from the group consisting of a phenol moiety, an aromatic ester moiety and an aromatic acid moiety, wherein the stabilized particles have a volume weighted median diameter (D50) of the particles from about 50 nm to about 500 nm, the complexing agent being present in an amount from about 0.05% to about 5%, w/w based on the weight of particles, the particles dispersed in an aqueous solution which further contains at least two preservatives in an amount sufficient to inhibit microbial growth. The hydrophilic polymer may be in an amount from about 3% to about 50%, w/w, based on the weight of the solid particles. The wetting agent may be an amount from about 0.01% to about 10%, w/w, based on the weight of the solid particles. The pregnenolone neurosteroid (e.g., ganaxolone) may be in an amount from about 10% to about 80% (and in certain embodiments form about 50% to about 80%) based on the weight of the stabilized particles. The stabilized particles may exhibit an increase in volume weighted median diameter (D50) of not more than about 150% when the particles are dispersed in simulated gastric fluid (SGF) or simulated intestinal fluid (SIF) at a concentration of 0.5 to 1 mg ganaxolone/mL and placed in a heated bath at 36° to 38° C. for 1 hour as compared to the D50 of the stabilized particles when the particles are dispersed in distilled water under the same conditions, wherein the volume weighted median diameter (D50) of the stabilized particles dispersed in SGF or SIF is less than about 750 nm. The stabilized particles may exhibit an increase in volume weighted median diameter (D50) of not more than about 150% when the formulation is dispersed in 15 mL of SGF or SIF at a concentration of 0.5 to 1 mg ganaxolone/mL as compared to the D50 of the stabilized particles when the particles are dispersed in distilled water under the same conditions, wherein the volume weighted median diameter (D50) of the stabilized particles dispersed in SGF or SIF is less than about 750 nm. The complexing agent may be a paraben, benzoic acid, phenol, sodium benzoate, methyl anthranilate, and the like. The hydrophilic polymer may be a cellulosic polymer, a vinyl polymer and mixtures thereof. The cellulosic polymer may be a cellulose ether, e.g., hydroxypropymethylcellulose. The vinyl polymer may be polyvinyl alcohol, e.g., vinyl pyrrolidone/vinyl acetate copolymer (S630). The wetting agent may be sodium lauryl sulfate, a pharmaceutically acceptable salt of docusate, and mixtures thereof. The aqueous dispersion may further comprise a sweetener, e.g., sucralose. The preservative is selected from the group consisting of potassium sorbate, methylparaben, propylparaben, benzoic acid, butylparaben, ethyl alcohol, benzyl alcohol, phenol, benzalkonium chloride, and mixtures of any of the foregoing.

In some embodiments, liquid pregnenolone neurosteroid (e.g., ganaxolone) formulations are provided comprising the ganaxolone particles described herein and at least one dispersing agent or suspending agent for oral administration to a subject. The ganaxolone formulation may be a powder and/or granules for suspension, and upon admixture with water, a substantially uniform suspension is obtained. As described herein, the aqueous dispersion can comprise amorphous and non-amorphous ganaxolone particles of consisting of multiple effective particle sizes such that ganaxolone particles having a smaller effective particle size are absorbed more quickly and ganaxolone particles having a larger effective particle size are absorbed more slowly. In certain embodiments the aqueous dispersion or suspension is an immediate release formulation. In another embodiment, an aqueous dispersion comprising amorphous ganaxolone particles is formulated such that about 50% of the ganaxolone particles are absorbed within about 3 hours after administration and about 90% of the ganaxolone particles are absorbed within about 10 hours after administration. In other embodiments, addition of a complexing agent to the aqueous dispersion results in a larger span of ganaxolone containing particles to extend the drug absorption phase such that 50-80% of the particles are absorbed in the first 3 hours and about 90% are absorbed by about 10 hours.

A suspension is “substantially uniform” when it is mostly homogenous, that is, when the suspension is composed of approximately the same concentration of pregnenolone neurosteroid (e.g., ganaxolone) at any point throughout the suspension. Preferred embodiments are those that provide concentrations essentially the same (within 15%) when measured at various points in a ganaxolone aqueous oral formulation after shaking. Especially preferred are aqueous suspensions and dispersions, which maintain homogeneity (up to 15% variation) when measured 2 hours after shaking. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

In some embodiments, the pregnenolone neurosteroid (e.g., ganaxolone) powders for aqueous dispersion described herein comprise stable ganaxolone particles having an effective particle size by weight of less than 500 nm formulated with ganaxolone particles having an effective particle size by weight of greater than 500 nm. In such embodiments, the formulations have a particle size distribution wherein about 10% to about 100% of the ganaxolone particles by weight are between about 75 nm and about 500 nm, about 0% to about 90% of the ganaxolone particles by weight are between about 150 nm and about 400 nm, and about 0% to about 30% of the ganaxolone particles by weight are greater than about 600 nm. The ganaxolone particles describe herein can be amorphous, semi-amorphous, crystalline, semi-crystalline, or mixture thereof.

In one embodiment, the aqueous suspensions or dispersions described herein comprise ganaxolone particles or ganaxolone complex at a concentration of about 20 mg/ml to about 150 mg/ml of suspension. In another embodiment, the aqueous oral dispersions described herein comprise ganaxolone particles or ganaxolone complex particles at a concentration of about 25 mg/ml to about 75 mg/ml of solution. In yet another embodiment, the aqueous oral dispersions described herein comprise ganaxolone particles or ganaxolone complex at a concentration of about 50 mg/ml of suspension. The aqueous dispersions described herein are especially beneficial for the administration of ganaxolone to infants (less than 2 years old), children under 10 years of age and any patient group that is unable to swallow or ingest solid oral dosage forms.

Liquid pregnenolone neurosteroid (e.g., ganaxolone) formulation dosage forms for oral administration can be aqueous suspensions selected from the group including, but not limited to, pharmaceutically acceptable aqueous oral dispersions, emulsions, solutions, and syrups. See, e.g., Singh et al., Encyclopedia of Pharmaceutical Technology, 2.sup.nd Ed., pp. 754-757 (2002). In addition to ganaxolone particles, the liquid dosage forms may comprise additives, such as: (a) disintegrating agents; (b) dispersing agents; (c) wetting agents; (d) at least one preservative, (e) viscosity enhancing agents, (f) at least one sweetening agent, (g) at least one flavoring agent, (h) a complexing agent. and (i) an ionic dispersion modulator. In some embodiments, the aqueous dispersions can further comprise a crystalline inhibitor.

Examples of disintegrating agents for use in the aqueous suspensions and dispersions include, but are not limited to, a starch, e.g., a natural starch such as corn starch or potato starch, a pregelatinized starch such as National 1551, or sodium starch glycolate such as PROMOGEL® (aloe vera extract, glycerin, alcohol) or EXPLOTAB® (sodium starch glycolate); a cellulose such as a wood product, microcrystalline cellulose, e.g., AVICEL® (microcrystalline cellulose (MCC)), AVICEL® PH10 (microcrystalline cellulose (MCC))1, AVICEL® PH102 (microcrystalline cellulose (MCC)), AVICEL® PH105 (microcrystalline cellulose (MCC)), ELCEMA® P100 (microcrystalline cellulose (MCC)), powdered cellulose, flour cellulose, microfine cellulose), EMOCEL® (microcrystalline cellulose, powdered cellulose), VIVACEL® (microcrystalline cellulose (MCC_), MING TIA® (microcrystalline cellulose (MCC)), and SOLKA FLOC® (powdered cellulose), methylcellulose, croscarmellose, or a cross-linked cellulose, such as cross-linked sodium carboxymethylcellulose (AC DI SOL® (croscarmellose sodium, internally cross-linked sodium carboxymethyl cellulose (NaCMC)), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as VEEGUM® (complex colloidal magnesium aluminum silicate), HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, TWEEN® (polyethylene glycol sorbitan monoacylate) 60 or 80, PEG, polyvinylpyrrolidone (PVP; commercially known as PLASDONE®), and the carbohydrate-based dispersing agents such as, for example, hydroxypropylcellulose and hydroxypropylcellulose ethers (e.g., HPC, HPC-SL, and HPC-L), hydroxypropylmethylcellulose and hydroxypropylmethylcellulose ethers (e.g. HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (PLASDONE®, e.g., S-630), 4-(1,1,3,3-tetramethylbutyl)-phenol polymer with ethylene oxide and formaldehyde (also known as tyloxapol), poloxamers (e.g., PLURONICS® (poly(ethylene oxide) and poly (propylene oxide) block copolymers) F68®, F88®, and F108®, which are block copolymers of ethylene oxide and propylene oxide); and poloxamines (e.g., Tetronic 9080, also known as Poloxamine 9080, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; TWEEN® (polyethylene glycol) 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and-PHARMACOAT® (hypromellose, hydroxypropyl methylcellulose) USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., PLURONICS F68®, F88®, and F108®, which are block copolymers of ethylene oxide and propylene oxide); or poloxamines (e.g., TETRONIC 908® (poloxamines, ethylenediamine tetrakis(propoxylate-block-ethoxylate) tetrol), also known as Poloxamine 908%).

Wetting agents (including surfactants) suitable for the aqueous suspensions and dispersions described herein are known in the art and include, but are not limited to, acetyl alcohol, glycerol monostearate, polyoxyethylene sorbitan fatty acid esters (e.g., the commercially available TWEEN® (polyethylene glycol) such as e.g., TWEEN20® and TWEEN80® (ICI Specialty Chemicals)), and polyethylene glycols (e.g., CARBOWAX 3350® and 1450® (polyethylene glycol), and CARBPOL 934 (cross-linked polyacrylic acid polymer), (Union Carbide), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben) and their salts, benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth. In one embodiment, the aqueous liquid dispersion can comprise methylparaben and propylparaben in a concentration ranging from about 0.01% to about 0.3% methylparaben by weight to the weight of the aqueous dispersion and 0.005% to 0.03% propylparaben by weight to the total aqueous dispersion weight. In yet another embodiment, the aqueous liquid dispersion can comprise methylparaben 0.05 to about 0.1 weight % and propylparaben from 0.01-0.02 weight % of the aqueous dispersion.

Suitable viscosity enhancing agents for the aqueous suspensions or dispersions described herein include, but are not limited to, methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity enhancing agent will depend upon the agent selected and the viscosity desired.

Examples of natural and artificial sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MAGNASWEET® (glycyrrhizic acid, ammonium salts), maltol, mannitol, maple, marshmallow, menthol, mint cream, mixed berry, neohesperidine DC, neotame, orange, pear, peach, peppermint, peppermint cream, PROSWEER® (fructose and glucose). Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.0001% to about 10.0% the weight of the aqueous dispersion. In another embodiment, the aqueous liquid dispersion can comprise a sweetening agent or flavoring agent in a concentration ranging from about 0.0005% to about 5.0% wt % of the aqueous dispersion. In yet another embodiment, the aqueous liquid dispersion can comprise a sweetening agent or flavoring agent in a concentration ranging from about 0.0001% to 0.1 wt %, from about 0.001% to about 0.01 weight %, or from 0.0005% to 0.004% of the aqueous dispersion.

In addition to the additives listed above, the liquid pregnenolone neurosteroid (e.g., ganaxolone) formulations can also comprise inert diluents commonly used in the art, such as water or other solvents, solubilizing agents, and emulsifiers.

In some embodiments, the pharmaceutical pregeneolone neurosteroid (e.g., ganaxolone) formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art include, but are not limited to, for example, U.S. Pat. Nos. 5,858,401, 6,667,048, and 6,960,563, each of which is specifically incorporated by reference.

Exemplary emulsifiers are ethyl alcohol, isopropyl alcohol, ethyl carbonate, ethyl acetate, benzyl alcohol, benzyl benzoate, propylene glycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In certain preferred embodiments, the liquid pharmaceutical formulation comprising ganaxolone, hydroxypropyl methylcellulose, polyvinyl alcohol, sodium lauryl sulfate, simethicone, methyl paraben, propyl paraben, sodium benzoate, citric acid, and sodium citrate at pH 3.8-4.2. The suspension may comprise ganaxolone at a concentration of 50 mg/ml. The formulation may further comprise a pharmaceutically acceptable sweetener (e.g., sucralose) and/or a pharmaceutically acceptable flavorant (e.g., cherry). The formulation may be enclosed, e.g., in a 120 mL, 180 mL, 240 mL, or 480 mL bottle.

In certain preferred embodiments, the oral solid formulation of the present invention may be a formulation as described and prepared in Applicant's prior U.S. Pat. No. 7,858,609, entitled “Solid Ganaxolone Formulations and Methods for the Making and Use Thereof”, hereby incorporated by reference in its entirety. However, the oral solid dosage formulation of pregnenolone neurosteroid (e.g., oral capsule or tablets) may be prepared in accordance with other methods known to those skilled in the art.

For example, as disclosed in U.S. Pat. No. 7,858,609, the oral solid formulation comprises stabilized particles comprising the pregenolone neurosteroid (e.g., ganaxolone), a hydrophilic polymer, a wetting agent, and an effective amount of a complexing agent that stabilizes particle growth after an initial particle growth and endpoint is reached, the complexing agent being a small organic molecule having a molecular weight less than 550 and containing a moiety selected from the group consisting of a phenol moiety, an aromatic ester moiety and an aromatic acid moiety, wherein the stabilized particles have a volume weighted median diameter (D50) of the particles is from about 50 nm to about 500 nm, the complexing agent being present in an amount from about 0.05% to about 5% w/w, based on the weight particles of the solid. The hydrophilic polymer may be in an amount from about 3% to about 50%, w/w, based on the weight of the solid particles. The wetting agent may be an amount from about 0.01% to about 10%, w/w, based on the weight of the solid particles. The pregnenolone neurosteroid (e.g., ganaxolone) may be in an amount from about 10% to about 80% (and in certain embodiments form about 50% to about 80%) based on the weight of the stabilized particles. The stabilized particles may exhibit an increase in volume weighted median diameter (D50) of not more than about 150% when the particles are dispersed in simulated gastric fluid (SGF) or simulated intestinal fluid (SIF) at a concentration of 0.5 to 1 mg ganaxolone/mL and placed in a heated bath at 36° to 38° C. for 1 hour as compared to the D50 of the stabilized particles when the particles are dispersed in distilled water under the same conditions, wherein the volume weighted median diameter (D50) of the stabilized particles dispersed in SGF or SIF is less than about 750 nm. The stabilized particles may exhibit an increase in volume weighted median diameter (D50) of not more than about 150% when the formulation is dispersed in 15 mL of SGF or SIF at a concentration of 0.5 to 1 mg ganaxolone/mL as compared to the D50 of the stabilized particles when the particles are dispersed in distilled water under the same conditions, wherein the volume weighted median diameter (D50) of the stabilized particles dispersed in SGF or SIF is less than about 750 nm. The solid stabilized particles may be combined with optional excipients and prepared for administration in the form of a powder, or they may be incorporated into a dosage form selected from the group consisting of a tablet or capsule. The complexing agent may be a paraben, benzoic acid, phenol, sodium benzoate, methyl anthranilate, and the like. The hydrophilic polymer may be a cellulosic polymer, a vinyl polymer and mixtures thereof. The cellulosic polymer may be a cellulose ether, e.g., hydroxypropymethylcellulose. The vinyl polymer may be polyvinyl alcohol, e.g., vinyl pyrrolidone/vinyl acetate copolymer (S630). The wetting agent may be sodium lauryl sulfate, a pharmaceutically acceptable salt of docusate, and mixtures thereof. When the particles are incorporated into a solid dosage form, the solid dosage form may further comprise at least one pharmaceutically acceptable excipient, e.g., an ionic dispersion modulator, a water soluble spacer, a disintegrant, a binder, a surfactant, a plasticizer, a lubricant, a diluent and any combinations or mixtures thereof. The water soluble spacer may be a saccharide or an ammonium salt, e.g., fructose, sucrose, glucose, lactose, mannitol. The surfactant may be, e.g., polysorbate. The plasticizer may be, e.g., polyethylene glycol. The disintegrant may be cross-linked sodium carboxymethylcellulose, crospovidone, mixtures thereof, and the like.

A capsule may be prepared, e.g., by placing the bulk blend pregnenolone neurosteroid (e.g., ganaxolone) formulation, described above, inside of a capsule. In some embodiments, the ganaxolone formulations (non-aqueous suspensions and solutions) are placed in a soft gelatin capsule. In other embodiments, the ganaxolone formulations are placed in standard gelatin capsules or non-gelatin capsules such as capsules comprising HPMC. In other embodiments, the ganaxolone formulations are placed in a sprinkle capsule, wherein the capsule may be swallowed whole or the capsule may be opened and the contents sprinkled on food prior to eating. In some embodiments of the present invention, the therapeutic dose is split into multiple (e.g., two, three, or four) capsules. In some embodiments, the entire dose of the ganaxolone formulation is delivered in a capsule form.

In certain embodiments, each capsule contains either 200 mg or 225 mg ganaxolone, and hydroxypropyl methylcellulose, sucrose, polyethylene glycol 3350, polyethylene glycol 400, sodium lauryl sulfate, sodium benzoate, citric acid anhydrous, sodium methyl paraben, microcrystalline cellulose, 30% Simethicone Emulsion, gelatin capsules, polysorbate 80, and sodium chloride. In some of the embodiments, the size of the capsule is 00.

Alternatively, the oral dosage forms of the present invention may be in the form of a controlled release dosage form, as described in U.S. Pat. No. 7,858,609.

The pregnenolone neurosteroid (e.g., ganaxolone) formulations suitable for use in the present invention may also be administered parenterally. In such embodiments, the formulations are suitable for intramuscular, subcutaneous, or intravenous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, Ganaxolone can be dissolved at concentrations of >1 mg/ml using water soluble beta cyclodextrins (e.g. beta-sulfobutyl-cyclodextrin and 2-hydroxypropylbetacyclodextrin). A particularly suitable cyclodextrin is a substituted-β-cyclodextrin is CAPTISOL® (cyckodextrin). Proper fluidity can be maintained, for example, by the use of a coating such as lecithin, by the maintenance of the required particle size in the case of dispersions, and by the use of surfactants. Ganaxolone formulations suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. Ganaxolone suspension formulations designed for extended release via subcutaneous or intramuscular injection can avoid first pass metabolism and lower dosages of ganaxolone will be necessary to maintain plasma levels of about 50 ng/ml. In such formulations, the particle size of the ganaxolone particles and the range of the particle sizes of the ganaxolone particles can be used to control the release of the drug by controlling the rate of dissolution in fat or muscle.

Particularly useful injectable formulations are disclosed in Applicant's U.S. Patent Publication No. 2017/0258812 (U.S. Ser. No. 15/294,135, filed Oct. 14, 2016), herein incorporated by reference in its entirety. Other useful injectable formulations of pregnenolone neurosteroids known to those skilled in the art can also be used.

### Combination Treatment

The disclosure includes embodiments in which the neurosteroid is the only active agent and embodiments in which the neurosteroid is administered in combination with one or more additional active agents. When used in combination with an additional active agent the neurosteroid and the additional active agent may be combined in the same formulation or may be administered separately. The neurosteroid may be administered while the additional active agent is being administered (concurrent administration) or may be administered before or after the additional active agent is administered (sequential administration).

The disclosure includes embodiments in which the additional active agent is an anti-convulsant. Anticonvulsants include GABA_{A} receptor modulators, sodium channel blocker, GAT-1 GABA transporter modulators, GABA transaminase modulators, voltage-gated calcium channel blockers, and peroxisome proliferator-activated alpha modulators.

The disclosure includes embodiments in which the patient is given an anesthetic or sedative in combination with a neurosteroid. The anesthetic or sedative may be administered at a concentration sufficient to cause the patient to lose consciousness, such as a concentration sufficient to medically induce coma or a concentration effective to induce general anesthesia. Or the anesthetic or sedative may be given at a lower dose effective for sedation, but not sufficient to induce a loss of consciousness.

Benzodiazepines are used both as anticonvulsants and anesthetics. Benzodiazepines useful as anaesthetics include diazepam, flunitrazepam, lorazepam, and midazolam.

In certain embodiments, neurosteroid is administered concominatly with a benzodiazepine (e.g., clobazam, diazepam, clonazepam, midazolam, clorazepic acid, Levetiracetam, felbamate, lamotrigine, a fatty acid derivative (e.g., valproic acid), a carboxamide derivative (rufinamide, carbamazepine, oxcarbazepine, etc.), an amino acid derivative (e.g., levocarnitine), a barbiturate (e.g., phenobarbital), or a combination of two or more of the foregoing agents.

The neurosteroid nanoparticle injectable formulation of this disclosure may be administered with another anticonvulsant agent. Anticonvulsants include a number of drug classes and overlap to a certain extent with the coma-inducing, anesthetic, and sedative drugs that may be used in combination with a neurosteroid. Anticonvulsants that may be used in combination with the neurosteroid nanoparticle injectable formulation of this disclosure include aldehydes, such as paraldehyde; aromatic allylic alcohols, such as stiripentol; barbiturates, including those listed above, as well as methylphenobarbital and barbexaclone; benzodiazepines include alprazolam, bretazenil, bromazepam, brotizolam, chloridazepoxide, cinolazepam, clonazepam, chorazepate, clopazam, clotiazepam, cloxazolam, delorazepam, diazepam, estazolam, etizolam, ethyl loflazepate, flunitrazepam, flurazepam, flutoprazepam, halazepam, ketazolam, loprazolam, lorazepam, lormetazepam, medazepam, midazolam, nimetazepam, nitrazepam, nordazepam, oxazepam, phenenazepam, pinazepam, prazepam, premazepam, pyrazolam, quazepam, temazepam, tatrazepam, and triazolam; bromides, such as potassium bromide; carboxamides, such carbamazepine, oxcarbazepine, and eslicarbazepine acetate; fatty acids, such as valproic acid, sodium valproate and divalproex sodium; fructose derivatives, such as topiramate; GABA analogs such as gabapentin and pregabalin, hydantoins, such as ethotoin, phenytoin, mephenytoin, and fosphenytoin; other neurosteroids, such as allopregnanolone, oxasolidinediones, such as paramethadione, trimethadione, and ethadione, propionates such as beclamide; pyrimidinediones such as primidone, pyrrolidines such as brivaracetam, levetiracetam, and seletracetam, succinimides, such as ethosuximide, pensuximide, and mesuximide; sulfonamides such as acetazoloamide, sultiame, methazolamide, and zonisamide; triazines such as lamotrigine, ureas such as pheneturide and phenacemide; NMDA antagonists, such as felbamate, and valproylamides such as valpromide and valnoctamide; and perampanel.

### Biomarker

Predictive biomarkers are used to identify patient populations that are more homogenous and have a higher propensity to respond to a therapy.

Allopregnanolone, a metabolite of progesterone, is a positive allosteric modulator (PAM) of the GABA_{A}-receptor with known anticonvulsive effects. A deficiency in this endogenous GABA_{A} modulator could result in a hyperexcitable neuronal network in the brain leading to an increased risk of seizures.

It is believed that most/all of those with PCDH19 mutations would exhibit this allopregnanolone deficiency supporting the hypothesis that treatment with ganaxolone may reduce seizure frequency and, possibly, improve additional symptoms of PCDH19.

Individuals affected by PCDH19-related epilepsy were found to exhibit an endogenous allopregnanolone deficiency when compared to age-matched controls (Tan C et al. 2015). The mechanism for this observation was attributed to a downregulation of the AKR1C2 and AKR1C3 genes which code for key enzymes responsible for steroidal metabolism resulting in allopregnanolone.

It has now been unexpectedly found that a way to identify high responders to neurosteroid treatment is through measurement of an endogenous neurosteroid (e.g., (allopregnanolone-sulfate; Allo-S) level(s) in patients. It is hypothesized that Allo-S is interrelated with allopregnanolone and may be the dominate analyte, between the two, in plasma. A low level of the endogenous neurosteroid may be used identify a patient population that potentially has a much higher response rate to ganaxolone treatment than those that have a high level of the endogenous neurosteroid.

Post-hoc review of baseline endogenous neurosteroid levels in the 11 PCDH19 subjects described in Example 11, yielded additional important observations. In these subjects, allopregnanolone sulfate (Allo-S) levels and 28-day seizure rates were assessed. A ganaxolone responder was specified, by post-hoc definition, as having at least a 25% reduction in 28-day seizure rate. In these 11 PCDH19 subjects, responders (n=6) and non-responders (n=5) had plasma Allo-S concentrations of 501±430 pg mL-1 and 9,829±6,638 pg mL-1, respectively, (mean±SD). There appeared to be a bimodal distribution of Allo-S plasma levels with one subset of subjects having a dramatically elevated level compared to the other (FIG. 10). At 6 months to baseline, the biomarker-positive group significantly improved (p=0.02, Wilcoxon) whereas the biomarker-negative (high Allo-S) group did not improve, but also did not significantly deteriorate (p=0.25, Wilcoxon) when comparing seizure frequency.

It was further discovered, when performing a retrospective separation of the PCDH19 cohort according to their Allo-S level, that the 7 subjects with Allo-S levels below 2,500 pg mL⁻¹ (G. Pinna Lab Method) had a 53.9% reduction in seizure rates while the 4 subjects with Allo-S levels above 2,500 pg mL⁻¹ had a 247% increase.

Thus, in certain embodiments of the present invention, allopregnanolone-sulfate (Allo-S) is used as a predictive biomarker for a response to ganaxolone, an analog of allopregnanolone. In these embodiments, Allo-S plasma level of 2,500 pg mL⁻¹ or less indicates that a subject is likely to respond and benefit from ganaxolone therapy; and a plasma level of Allo-S plasma level of above 2,500 pg mL⁻¹ indicates that a subject is unlikely to respond to ganaxolone therapy and that a different therapeutic agent should be used. Administering ganaxolone to subjects with Allo-S plasma level of 2,500 pg mL⁻¹ or less could restore a normal neuronal network in these subjects and decrease seizure frequency.

### DETAILED DESCRIPTION OF PREFERRED EMBODIMENTS

The following examples of formulations in accordance with the present invention are not to be construed as limiting the present invention in any manner and are only samples of the various formulations described herein.

During the development of ganaxolone formulations, a variety of formulations have been evaluated to establish a formulation that demonstrates adequate pharmacokinetic (“PK”) parameters and is suitable for development and commercialisation. Other formulations of ganaxolone used included ganaxolone mixed with sodium lauryl sulfate, with hydroxypropyl-beta-cyclodextrin (HP-β-CD) in solution, and with beta cyclodextrin (0-CD) administered as a variety of suspensions, as well as ganaxolone 0.5 micron particles in suspension and tablet formulations, and controlled-release capsule formulations, and an IV solution using sulfobutylether cyclodextrin (CAPTISOL® (cyclodextrin)) for solubilization of ganaxolone. The development effort led to oral suspension comprising 0.3 micron immediate release particles of ganaxolone, which is described in Example 1, and an oral capsule formulation comprising 0.3 micron immediate release particles of ganaxolone, which is described in Example 2. The pharmacokinetics of these formulations of ganaxolone in humans has been investigated in a number of single- and multiple-dose studies in adults. The results of these studies are summarized in Examples 6 and 7.

### Example 1

A 50 mg/mi ganaxolone suspension is prepared having the ingredients set forth in Table 1 below:

TABLE 1
Composition of 50 mg/ml Ganaxolone Suspension

| Ingredient | Grade | % w/w | mg/ml |
|---|---|---|---|
| Ganaxolone | GMP | 4.91 | 50.0 |
| Hypromellose (Pharmacoat | USP/EP | 5.0 | 50.91 |
| 603) | | | |
| Polyvinyl alcohol | USP/EP | 1.0 | 10.18 |
| Sodium lauryl sulfate | USP/EP | 0.1 | 1.02 |
| Methylparaben | NF/EP | 0.1 | 1.02 |
| Propylparaben | NF/EP | 0.02 | 0.20 |
| Sodium benzoate | USP/EP | 0.09 | 0.92 |
| Citric acid, anhydrous | USP/EP | 0.12 | 1.22 |
| Sodium citrate dihydrate | USP/EP | 0.0093 | 0.095 |
| Cherry artificial flavor | Pharmaceutical | 0.0025 | 0.025 |
| Firmenich No. 57679 A | | | |
| Sucralose | NF | 0.02 | 0.20 |
| 30% Simethicone emulsion, | USP | 0.0333 | 0.34 |
| (Dow Corning Q7-2587) | | | |
| Purified water | USP | q.s. 100.0 | q.s. 1.0 mL |

Table 2 shows the function of the excipients used in the 50 mg/ml ganaxolone suspension.

TABLE 2
Summary of Ingredient Function of
the 50 mg/ml Ganaxolone Suspension

| Ingredient | Function |
|---|---|
| Ganaxolone | Active Pharmaceutical |
| | Ingredient |
| Hypromellose (Pharmacoat 603), | Polymeric nanoparticle |
| USP/EP | steric stabilizer |
| Sodium Lauryl Sulfate, USP, EP, NF | Anionic nanoparticle |
| | electrostatic stabilizer |
| 30% Simethicone Emulsion (Dow | Anti-foaming agent |
| Corning Q7-2587) | |
| Methylparaben USP/NF | Nanoparticle stabilizer & |
| | antimicrobial preservative |
| Sodium Benzoate | Nanoparticle stabilizer & |
| | antimicrobial preservative |
| Citric Acid Anhydrous, USP/EP | pH adjustment |
| Propylparaben NF | Nanoparticle stabilizer & |
| | antimicrobial preservative |
| Sodium Citrate Dihydrate, USP/FCC | pH adjustment |
| Polyvinyl Alcohol 4-88; Emprove ® | stabilizer |
| exp PhEur, USP, JPE | |
| Sucralose Powder, NF (micronized) | Sweetener |
| Artificial Cherry Flavor (Firmenich | Flavor |
| 502068 C) | |
| Purified Water USP/EP | diluent |

The oral bioavailability of the 50 mg/ml ganaxolone suspension is dependent upon the rate and extent of nanoparticulate drug dissolution in the relevant physiological environment. The particle sizing method and specification is intended to ensure that ganaxolone drug product exhibits an absence of agglomeration following dispersal in simulated gastrointestinal fluids.

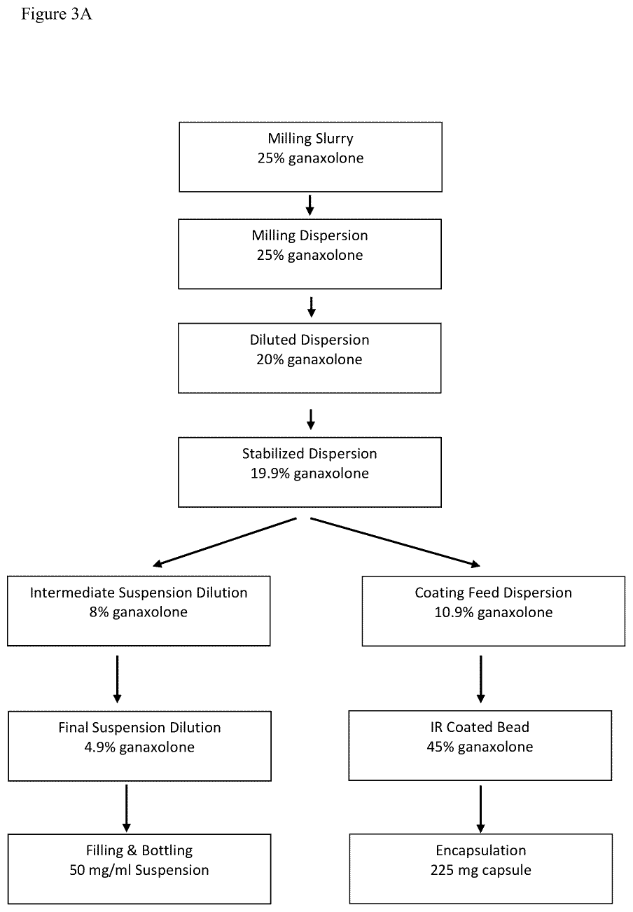

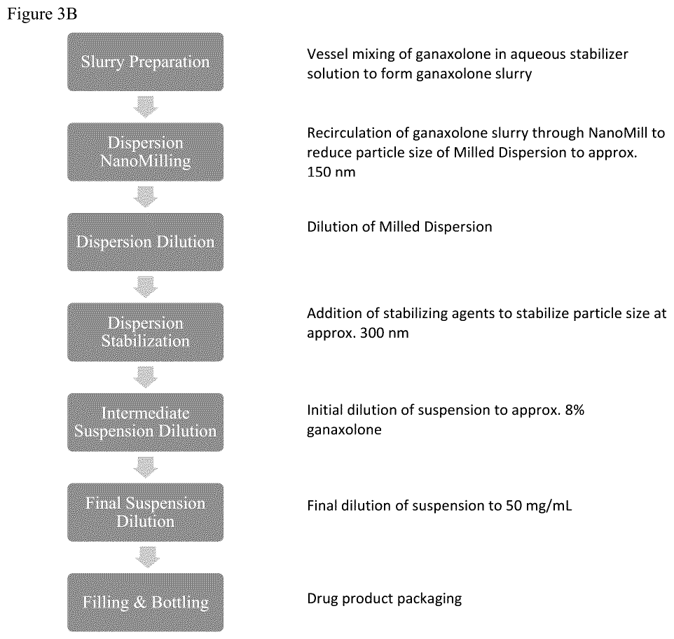

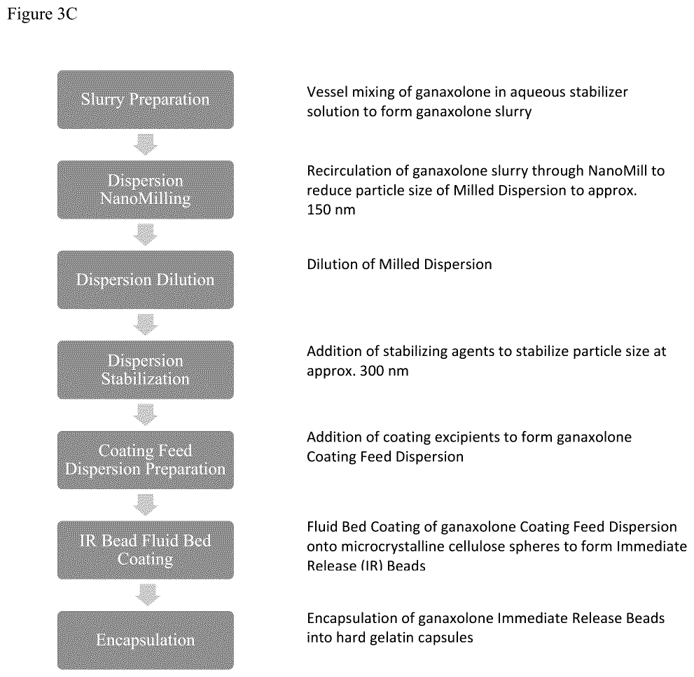

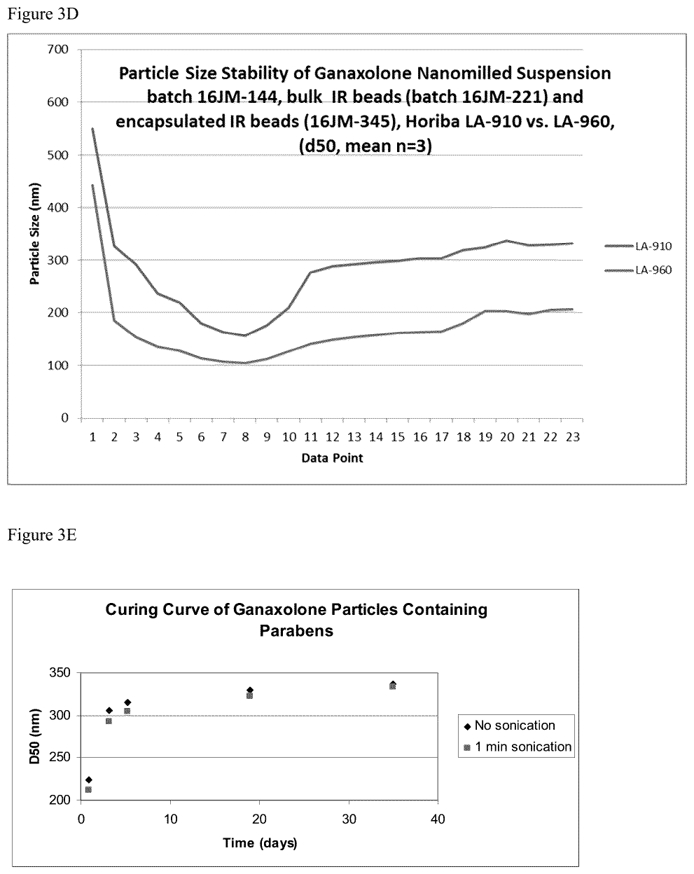

FIG. 3B provides a summary of the key steps in the suspension manufacturing process that apply to the 50 mg/ml ganaxolone suspension.

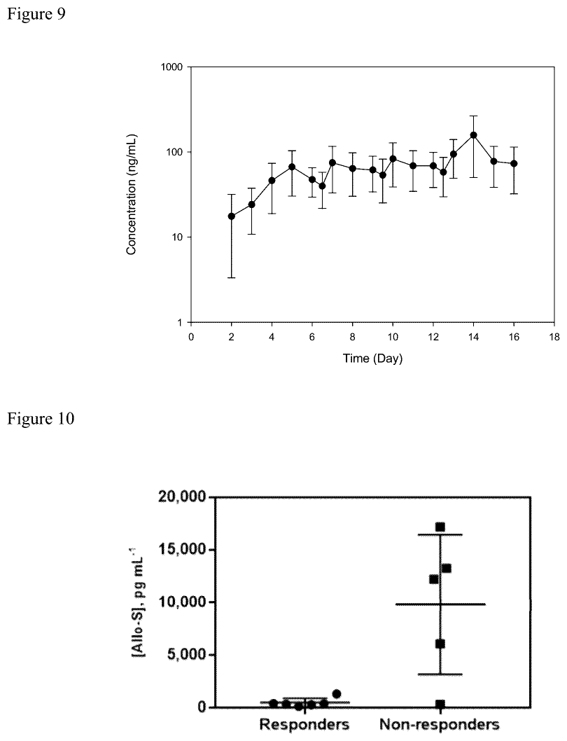

A dispersion nanomilling process is used to reduce the particle size of ganaxolone and obtain stable ganaxolone nanoparticles. The nanomilling process includes the use of yttria-stabilized zirconia (YTZ) milling media under high-energy agitation within the nanomill. In order to ensure a consistent slurry particle size prior to dispersion nanomilling, Marinus has developed a high-energy rotor/stator premilling process using a VakuMix DHO-1. Following nanomilling, the dispersion is diluted from 25% w/w ganaxolone to 20% w/w ganaxolone and filtered through a 20-micron filter, and stabilizing agents (methylparaben, sodium benzoate and citric acid anhydrous) are added to promote controlled growth during a 5-10-day curing period at room temperature to approximately 300 nm. FIG. 9 illustrates a typical particle size growth profile. The stabilized 300 nm nanoparticles exhibit good stability against particle growth in pediatric suspension drug product and encapsulated drug product formats. The stabilization process is controlled by accurate addition and dissolution of parabens, which are water soluble stabilization agents. The curing process is controlled by regulation of hold time and temperature of the stabilized dispersion prior to suspension dilution (in the case of 50 mg/ml ganaxolone suspension) or fluid bed bead coating (in the case of the 225 mg ganaxolone capsules described in Example 2).

Three dispersion batches prepared in the dispersion nanomilling scale-up study were diluted and stabilized with the addition of sodium methylparaben, sodium benzoate and citric acid anhydrous and cured for 7 days. After curing, the particle size was measured and is shown in Table 3.

TABLE 3
Stabilized Dispersion Particle Size after 7 Days Curing

| Batch | D(10) (nm) | D(50) (nm) | D(90) (nm) |
|---|---|---|---|
| Dispersion Batch 1 | 212 | 298 | 689 |
| Dispersion Batch 2 | 208 | 289 | 539 |
| Dispersion Batch 3 | 209 | 291 | 498 |

D = diameter

As shown, the D(50) particle size was stabilized within the specification of 250-450 nm.

### Example 2

Ganaxolone capsules (225 mg) are prepared having the ingredients set forth in Tables 4 and 5 below:

TABLE 4
Composition of 225 mg Ganaxolone Capsule IR Bead

| Ingredient | Grade | % w/w |
|---|---|---|
| Ganaxolone | GMP | 45.06 |
| Hypromellose (Pharmacoat 603) | USP/EP | 10.28 |
| Sodium lauryl sulfate | USP/EP/NF | 0.70 |
| Methylparaben Sodium | USP | 0.26 |
| Sodium benzoate | USP/EP | 0.20 |
| Citric acid, anhydrous | USP/EP | 0.39 |
| Sodium Chloride | USP/EP | 1.03 |
| 30% Simethicone emulsion, (Dow Corning | USP | 0.11 |
| Q7-2587) | | |
| Sucrose - extra fine granulated | EP/NF | 23.04 |
| Polyethylene Glycol 3350 | NF/EP | 1.08 |
| Polyethylene Glycol 400 | NF/EP | 0.54 |
| Polysorbate 80 | NF/EP, JP | 0.65 |
| Microcrystalline Cellulose Spheres, Grade: | NF/EP | 16.65 |
| CP-305 | | |
| Total | | 100.0 |

Table 5 summarizes the function of the excipients used in the 225 mg ganaxolone capsule formulation.

TABLE 5
Summary of Ingredient Function of
the 225 mg Ganaxolone Capsule

| Ingredient | Function |
|---|---|
| Ganaxolone | Active Pharmaceutical |
| | Ingredient |
| Hypromellose (Pharmacoat 603) | Polymeric nanoparticle |
| USP/EP | steric stabilizer |
| Sodium Lauryl Sulfate USP, EP, NF | Anionic nanoparticle |
| | electrostatic stabilizer |
| 30% Simethicone Emulsion USP (Dow | Anti-foaming agent |
| Corning Q7-2587) | |
| Sodium Methylparaben (Nipagin M | Nanoparticle stabilizer & |
| Sodium) | antimicrobial preservative |
| Sodium Benzoate USP/EP | Nanoparticle stabilizer & |
| | antimicrobial preservative |
| Citric Acid Anhydrous USP/EP | pH adjustment |
| Sucrose | Binder/filler |
| Sodium Chloride | Ionic strength modifier |
| Polyethylene Glycol 3350 | Plasticizer |
| Polyethylene Glycol 400 | Plasticizer |
| Polysorbate 80 | Nonionic surfactant, |
| | stabilizer |
| Microcrystalline Cellulose Spheres | IR bead core |
| (Celphere CP305) | |
| Hard Gelatin Capsule, Size 00 | Dosage form capsule |

IR = Immediate Release

FIG. 3C provides a summary of the key steps in the suspension manufacturing process that apply to the 225 mg ganaxolone capsules. The manufacturing process used for the preparation of these capsules utilizes the same drug product specifications and the same quantitative compositions, and the same nanomilling dispersion dilution and dispersion stabilization processes. Thus, the product of Example 2 utilizes a common stabilized dispersion intermediate with the product of Example 1. The methylparaben sodium may be substituted with methylparaben.

Table 5A summarizes results of thirty-six month formal stability data of ganaxolone immediate release (IR) 225 mg Capsule:

TABLE 5A
Thirty-Six Month Formal Stability Data of Ganaxolone Immediate Release (IR) 225 mg Capsule (25° C./60% RH)

| | | | 25° C./60% RH | | | | | | | |
|---|---|---|---|---|---|---|---|---|---|---|
| Test | Specifications | Initial | 1 month | 3 month | 6 month | 9 month | 12 month | 18 Month | 24 Month | 36 Month |
| Assay (% | 90-110% LC | 101.4 | 100.9 | 100.3 | 99.2 | 99.8 | 99.3 | 100.6 | 100.6 | 98.4 |
| Label Claim) | | | | | | | | | | |
| Dissolution | NLT Q = 80% | 95% | 94% | 90% | 89% | 92% | 94% | 86% | 91% | 87% |
| | at 45 | | | | | | | | | |
| | minutes | | | | | | | | | |
| Profile | Report | 74, 84, | 79, 79, | 80, 85, | 81, 79, | 78, 82, | 90, 63, | 69, 80, | 66, 62, | 57, 58, 42, |
| 15 min | Results | 85, 88, | 77, 82, | 81, 70, | 77, 63, | 88, 86, | 76, 83, | 71, 70, | 84, 55, | 74, 35, 64, |
| | | 87, 86 | 82, 92 | 83, 78 | 72, 73 | 82 | 70, 88 | 72, 77, | 64, 74 | 60, 60, 47, |
| | | | | | | | | 68, 89, | | 27, 79, 60 |
| | | | | | | | | 48, 79, | | |
| | | | | | | | | 70, 73 | | |
| Profile | Report | 93, 92, | 92, 94, | 86, 89, | 91, 87, | 88, 88, | 95, 88, | 80, 86, | 84, 83, | 75, 78, 81, |
| 30 min | Results | 92, 94, | 86, 88, | 88, 89, | 87, 80, | 90, 92, | 85, 91, | 77, 76, | 92, 83, | 83, 66, 85, |
| | | 96, 93 | 88, 97 | 85, 88 | 81, 86 | 94, 92 | 83, 93 | 78, 85, | 87, 89 | 79, 71, 86, |
| | | | | | | | | 83, 90, | | 60, 86, 85 |
| | | | | | | | | 76, 88, | | |
| | | | | | | | | 81, 87 | | |
| Profile | NLT 80% | 96, 94, | 94, 96, | 90, 90, | 93, 90, | 88, 90, | 95, 95, | 84, 88, | 89, 90, | 82, 88, 87, |
| 45 min | | 94, 95, | 92, 93, | 88, 91, | 89, 85, | 94, 93, | 95, 92, | 79, 83, | 92, 90, | 88, 83, 90, |
| | | 95, 95 | 93, 96 | 91, 90 | 85, 90 | 96, 94 | 91, 97 | 80, 86, | 91, 93 | 89, 79, 93, |
| | | | | | | | | 88, 91, | | 84, 90, 91 |
| | | | | | | | | 85, 90, | | |
| | | | | | | | | 88, 88 | | |
| Profile | Report | 94, 92, | 95, 95, | 92, 90, | 94, 91, | 88, 92, | 95, 94, | 85, 88, | 93, 93, | 91, 92, 88, |
| 60 min | Results | 95, 95, | 94, 93, | 89, 90, | 90, 85, | 94, 93, | 94, 94, | 80, 85, | 94, 96, | 89, 92, 91, |
| | | 95, 96 | 95, 95 | 91, 89 | 86, 91 | 95, 93 | 91, 97 | 84, 85, | 93, 93 | 82, 91, 88, |
| | | | | | | | | 90, 91, | | 90, 92 |
| | | | | | | | | 89, 91, | | |
| | | | | | | | | 89, 88 | | |
| Particle Size | 250-450 nm | 339 nm | 354 nm | 336 nm | 339 nm | 335 nm | 344 nm | 368 nm | 348 nm | 360 nm |
| (D50) nm | volume | | | | | | | | | |
| | weighted | | | | | | | | | |
| | median | | | | | | | | | |
| | diameter | | | | | | | | | |
| | (D50) | | | | | | | | | |

### Example 3

Example 3 concerns a Phase 2 Multicenter, Open-Label Proof-of-Concept Trial of ganaxolone (GNX) in cohorts of children having genetic epilepsies (PCDH19, CDKL5 LGS, and CSWS) (ClinicalTrials.gov Identifier: NCT02358538). There were 11 female children with PCDH19 epilepsy between 5-16 years old with a confirmed genetic mutation. There were 6 female and 1 male children with confirmed genetic mutations in the CDKL5 cohort. There were 10 children in the Lennox Gastaut Syndrome cohort. Two children with CSWS were enrolled into the study. The study was conducted with 12 weeks baseline, and up to 26 weeks of treatment followed by a 52 week open label treatment. The primary efficacy was the percentage change in seizure frequency per 28 days relative to baseline calculated using daily seizure diary. [Time Frame: 26 weeks]. Secondary Outcome Measures were: Clinician Global Impression of Change score as assessed by questionnaire. [Time Frame: 26 Weeks]; Patient Global Impression of Change score as assessed by questionnaire. [Time Frame: 26 Weeks]; Evaluation of safety and tolerability of open-label ganaxolone as adjunctive therapy for uncontrolled seizures in children with rare genetic epilepsies, based on adverse event log and other clinical safety assessments. [Time Frame: 26 weeks]; Responder rates [Time Frame: 26 weeks]; and Seizure free days [Time Frame: 26 weeks].

As noted in Table 6, across multiple placebo controlled studies of ganaxolone across multiple indications including epilepsy, few side effects are reported that occur at a rate higher than those reported in placebo-treated subjects. These side effects are generally mild and have always been reversible. Compared to other available therapies, ganaxolone has been shown to be generally safe and well-tolerated and a safe long term option for those children with good seizure control. Four of the 7 children enrolled into this study remain on ganaxolone. Adverse events in this study are similar to those reported for all placebo controlled studies completed to date as summarized in Table 6 below.

TABLE 6
Integrated Table of Adverse Events from Placebo Controlled
Studies of Ganaxolone (≥5% for Ganaxolone)

| AE | Ganaxolone (n = 750) | Placebo (n = 540) |
|---|---|---|
| Somnolence | 134 (18%) | 31 (6%) |
| Dizziness | 95 (13%) | 24 (4%) |
| Fatigue | 51 (7%) | 21 (4%) |
| Headache | 37 (5%) | 28 (5%) |

Following screening and baseline evaluations, consenting patients enrolled into a 26-week study during which investigators were allowed to flexibly dose ganaxolone up to a dose of 1,800 mg/day for patients whose body weight was >30 kg, or up to 63 mg/kg/day for patients whose body-weight was <30 kg. The primary efficacy measure is % change from baseline in the 28-day seizure frequency count. Safety and tolerability were within the secondary objectives of the study.

In this study, oral ganaxolone suspension or capsules were administered up to a total of 63 mg/kg/day (maximum 1800 mg/day) over 2-4 weeks. About six-eight titration steps are used, depending on the size of the patient. Children larger than 30 kg may take the ganaxolone capsules. The ganaxolone oral suspension was administered through an oral dosing syringe three times daily. The ganaxolone capsules were administered twice daily. The patients experience better absorption of the ganaxolone with meals (milk).

Table 7 provides the suggested titration schedule by weight for ganaxolone oral suspension.

TABLE 7
Suggested Titration Schedule by Weight
for Ganaxolone Oral Suspension

| | | | | | | |
|---|---|---|---|---|---|---|
| | | | Dose | Total | % | Total |
| Titration | | Dose | mg/kg | mg | Dose | ml |
| Step # | mg/kg | (mg) | increase | change | Change | Suspension |

15 kg (33 lbs)

| | | | | | | |
|---|---|---|---|---|---|---|
| 1 | 18 | 270 | | | | 5.4 |
| 2 | 24 | 359 | 6 | 89 | 33% | 7.2 |
| 3 | 32 | 478 | 8 | 119 | 33% | 9.6 |
| 4 | 42 | 635 | 11 | 158 | 31% | 12.7 |
| 5 | 54 | 810 | 12 | 175 | 27% | 16.2 |
| 6 | 63 | 945 | 9 | 135 | 16% | 18.9 |

20 kg (44 lbs)

| | | | | | | |
|---|---|---|---|---|---|---|
| 1 | 18 | 360 | | | | 7.2 |
| 2 | 24 | 479 | 6 | 119 | 33% | 9.6 |
| 3 | 32 | 637 | 8 | 158 | 33% | 12.7 |
| 4 | 42 | 847 | 10 | 210 | 31% | 16.9 |
| 5 | 54 | 1080 | 12 | 233 | 27% | 21.6 |
| 6 | 63 | 1260 | 9 | 180 | 16% | 25.2 |

25 kg (55 lbs)

| | | | | | | |
|---|---|---|---|---|---|---|
| 1 | 18 | 450 | | | | 9.0 |
| 2 | 24 | 599 | 6 | 149 | 33% | 12.0 |
| 3 | 32 | 796 | 8 | 198 | 33% | 15.9 |
| 4 | 42 | 1059 | 10 | 263 | 31% | 21.2 |
| 5 | 54 | 1350 | 12 | 291 | 27% | 27.0 |
| 6 | 63 | 1575 | 9 | 225 | 16% | 31.5 |

30 kg (66 lbs)

| | | | | | | |
|---|---|---|---|---|---|---|
| 1 | 18 | 540 | | | | 10.8 |
| 2 | 24 | 718 | 6 | 178 | 33% | 14.4 |
| 3 | 32 | 955 | 8 | 237 | 33% | 19.1 |
| 4 | 42 | 1270 | 10 | 315 | 31% | 25.4 |
| 5 | 50 | 1500 | 8 | 230 | 18% | 30.0 |
| 6 | 55 | 1650 | 5 | 150 | 11% | 33.0 |
| 7 | 60 | 1800 | 5 | 150 | 9% | 36.0 |

Table 8 provides the suggested titration schedule by weight for ganaxolone oral capsules.

TABLE 8
Tanaxolone capsule for subjects ≥30 kg

| | 200 mg capsules | | | 225 mg capsules | | |
|---|---|---|---|---|---|---|
| | Total | No. | No | Total | No | No |
| Titration | Daily | Cap | Caps | Daily | Caps | Caps |
| Step | Dose | AM | PM | Dose | AM | PM |
| 1 | 400 | 1 | 1 | 450 | 1 | 1 |
| 2 | 600 | 1 | 2 | 675 | 1 | 2 |
| 3 | 800 | 2 | 2 | 900 | 2 | 2 |
| 4 | 1000 | 2 | 3 | 1125 | 2 | 3 |
| 5 | 1200 | 3 | 3 | 1350 | 3 | 3 |
| 6 | 1400 | 3 | 4 | 1575 | 3 | 4 |
| 7 | 1600 | 4 | 4 | 1800 | 4 | 4 |
| 8 | 1800 | 4 | 5 | | | |

As with CDKL5 Deficiency Disorder patients, an anti-epileptic treatment effect signal of ganaxolone has emerged in the PCDH19 cohort in this Phase 2 open-label study of ganaxolone in children with rare genetic epilepsies with uncontrolled seizures despite multiple previous and concurrent AED regimens. The preliminary data from 11 PCDH19 patients showed 9 of 11 patients with some degree of seizure reduction, with 4 patients achieving greater than 50% seizure reduction that persisted for greater than 6 months. Two patients completed 78 weeks on ganaxolone and are now receiving ganaxolone under an investigator sponsored IND. Although not presented, the CGI-I rated by clinician and parent/caregiver showed improvement consistent with seizure control.

Preliminary data is presented in Table 9.

TABLE 9

| | 28-day seizure | 28-day seizure | 28-day seizure | 28-day seizure |
|---|---|---|---|---|
| | rate % change | rate % change | free days % change | free days % change |
| Subject | at 3 months | at 6 months | at 3 months | at 6 months |
| 1 | −100.00 | rash | 23 | — |
| 2 | −78.83 | sz returned | 39 | — |
| 3 | −74.17 | −73.47 | 880 | 890 |
| 4 | −52.30 | −53.85 | 4 | 5 |
| 5 | −33.29 | −33.22 | 8 | 7 |
| 6 | 31.36 | −25.68 | 18 | 27 |
| 7 | −7.37 | −5.26 | 18 | 14 |
| 8 | −4.38 | −2.56 | 49 | 32 |
| 9 | 106.67 | 140.00 | −7 | −3 |
| 10 | 353.25 | early term | −19 | — |
| 11 | 1020.50 | early term | −89 | — |

Narratives describing the clinical status of patients from investigators indicate that some children treated with ganaxolone appeared to have meaningful improvement in non-seizure related problems.

According to a doctor who has treated 5 subjects with CDKL5 Deficiency Disorder, all his subjects have benefited from treatment in some manner, such as decreased seizure frequency, decreased seizure severity and/or increased attention associated with a calmer demeanour.

Based upon known mechanism of action, preclinical and clinical data, and narrative reports from the investigators, ganaxolone has the potential to address seizure and non-seizure related problems including anxiety, poor social interaction, motor deficits and poor sleep, all of which are common and severely disabling in children with CDKL5 deficiency disorder.

Adverse events possibly associated with the ganaxolone treatment are provided in Table 10 below:

TABLE 10

| | PCDH19 |
|---|---|
| | N = 11 |
| Event | N (%) |
| Somnolence | 4 (36.4) |
| Headache | 3 (27.3) |
| Seizure | 3 (27.3) |
| Fatigue | 3 (27.3) |
| Pyrexia | 2 (18.2) |
| Abdominal pain | 2 (18.2) |
| Vomiting | 2 (18.2) |

Of the 4 completed CDKL5 patients, 3 out of 4 showed a >50% reduction in their seizures counts: 52%, 59%, and 88%, respectively, and 2 out of 4 showed a marked improvement in seizure free days (78%, 368%). The Connor's Global Index for Investigators (CGI-I) and Parents (CGI-P) showed improvement consistent with seizure control. One patient discontinued due to lack of seizure control; however, caregiver reliability was questioned by the investigator. The safety and tolerability profile seen in these patients was consistent with earlier studies.

The preliminary data from the first 6 CDKL5 patients showed improvement in seizure control that persists for up to 6 months in 3 of 6 patients. The seventh patient who was recently added to the study was experiencing substantial seizure reduction after the first 28 days of treatment. Four of the 7 patients also had an increase in the number of seizure-free days. Although not presented, the Clinical Global Impression Improvement Scale (CGI-I) rated by clinician and parent/caregiver showed improvement consistent with seizure control. All of the subjects benefited from treatment in some manner, such as decreased seizure frequency, decreased seizure severity and/or increased attention associated with a calmer demeanor. Similar reports of increased social interaction, reduced seizure severity and duration and increased attention have been reported for children with PCDH19 and Lennox Gastaut Syndrome, further affirming the need to capture these important endpoints in the next clinical study of ganaxolone in CDKL5 Deficiency Disorder. One child with PCDH19 mutation was severely autistic and non-verbal prior to ganaxolone treatment. After she began ganaxolone treatment, her social interaction and verbal language were markedly improved (as documented by video as a reference. Such videos may be an important aspect to documenting change in functional ability in these children during ganaxolone treatment).

Table 11 provides the steroid and neurosteroid levels of the top 3 high responders versus the 3 worst non-responders. High responders had >70% seizure reduction; non-responders had >100% increase in seizures. One high responder and one non-responder had baseline values only so the baseline values were used for both baseline and 26-week timepoints.

TABLE 11

| | High Responder | | Marked Non-responder | |
|---|---|---|---|---|
| | Mean pg/mL | | Mean pg/mL | |
| (Neuro)Steroid | Baseline | 26 weeks | Baseline | 26 weeks |
| Pregnenolone | 1064 | 966 | 1670 | 1968 |
| Pregnenolone-S | 77 | 847 | 571 | 5829 |
| 5-alphaDHP | 999 | 1158 | 93 | 4048 |
| Allopregnanolone | 56 | 72 | 67 | 183 |
| Allopregnanolone-S | 704 | 1780 | 11851 | 12676 |
| Pregnanolone | 23 | 14 | 0 | 15 |
| Pregnanolone-S | 407 | 94 | 0 | 0 |
| DHEA | 806 | 998 | 631 | 1608 |

These results indicate that those patients who go on to have extremely high response rates of up to 100% reduction in seizures have considerably lower background plasma neurosteroids except for pregnanolone and pregnanolone sulfate which may actually be competitive with allopregnanolone for GABA_{A} binding sites unlike the others. This particular pattern of high levels of plasma neurosteroids continues through the 26 weeks of treatment with ganaxolone. This means that patients with very high background levels of neurosteroids, particularly allopregnanolone and especially allopregnanolone sulfate can be predicted to respond poorly to allopregnanolone, ganaxolone or other pregnanolone-based therapies. This finding enables the use of pregnanolone-based therapies such as ganaxolone to be directed preferentially to those patients with low background neurosteroid levels, especially allopregnanolone and allopregnanolone sulfate as they could be the most likely to respond and to a high degree with respect to seizure reduction and overall control of epilepsy.

In subjects with focal onset seizure disorder, a post hoc analysis showed a statistically significant reduction in seizure frequency for those subjects on ganaxolone taking 3 or more concomitant anti-epileptic drugs (AEDs) compared to those receiving placebo (ClinicalTrials.gov Identifier: NCT02358538). Ganaxolone was associated with a 20% greater reduction in median seizure frequency than placebo, p=0.02 (Lappalainen J, Tsai J, Amerine W, Patroneva. A Multicenter, Double-Blind, Randomized, Placebo-Controlled Phase 3 Trial to Determine the Efficacy and Safety of Ganaxolone as Adjunctive Therapy for Adults with Drug-Resistant Focal-Onset Seizures Neurology 2017:88, 16 Supplement P5.237). Although numerically superior, there was no statistically significant effect of ganaxolone compared to placebo for those subjects taking fewer than 3 AEDs. These data indicate the efficacy of ganaxolone to treat the most refractory of patients with epilepsy who require the most intensive medication regimens. Patients with CDKL5 deficiency disorder are nearly universally refractory to all available AEDs despite treatment with multiple concomitant medications.

Based on the results obtained to date, ganaxolone has demonstrated a good long-term safety and tolerability profile in children with this severe and currently untreatable disorder. As noted in Table 12, the median percent reduction in seizures of 43% and 34% for children with CDKL5 and PCDH19 disorders respectively, indicates the potential for ganaxolone to be a substantial improvement over existing therapies for children with severe, refractory, pediatric genetic epileptic encephalopathy, particularly CDKL5 Deficiency Disorder.

TABLE 12

| cohort | Variable | Label | N | Mean | Std Dev | Median | Minimum | Maximum |
|---|---|---|---|---|---|---|---|---|
| CDKL5 | basertsz | Pre-baseline 28-day seizure rate | 7 | 1171.06 | 2111.09 | 115.71 | 33.67 | 5702.06 |
| | seize28_3 | Post-baseline 28-day seizure rate-3-month | 7 | 821.16 | 1890.36 | 57.87 | 34.88 | 5101.29 |
| | pctchg_3 | % change seizure rate-3 month | 7 | −32.67 | 45.99 | −49.99 | −82.49 | 36.77 |
| | seize28_6 | Post-baseline 28-day seizure rate-6 month | 7 | 1373.85 | 3392.34 | 67.29 | 34.88 | 9065.28 |
| | pctchg_6 | % change seizure rate-6 month | 7 | −16.81 | 73.28 | −47.80 | −87.54 | 99.88 |
| | seize28_12 | Post-baseline 28-day seizure rate-12 month | 3 | 114.39 | 79.94 | 76.54 | 60.41 | 206.23 |
| | pctchg_12 | % change seizure rate-12 month | 3 | −56.12 | 29.95 | −47.79 | −89.35 | −31.22 |
| PCDH19 | basertsz | Pre-baseline 28-day seizure rate | 11 | 38.43 | 34.36 | 19.12 | 4.67 | 112.00 |
| | seize28_3 | Post-baseline 28-day seizure rate-3-month | 11 | 84.51 | 206.19 | 15.81 | 0.00 | 704.00 |
| | pctchg_3 | % change seizure rate-3 month | 11 | 105.59 | 328.87 | −7.37 | −100.00 | 1020.50 |
| | seize28_6 | Post-baseline 28-day seizure rate-6 month | 11 | 84.41 | 206.27 | 15.81 | 0.00 | 704.00 |
| | pctchg_6 | % change seizure rate-6 month | 11 | 103.72 | 330.72 | −25.68 | −100.00 | 1020.50 |
| | seize28_12 | Post-baseline 28-day seizure rate -12 month | 7 | 24.25 | 17.69 | 19.44 | 3.92 | 49.67 |
| | pctchg_12 | % change seizure rate-12 month | 7 | 51.34 | 155.25 | −8.57 | −76.60 | 353.25 |

These preliminary data compare very favorably with the outcomes cited in Müller A et al (Müller A, Helbig I, Jansen C, Bast T, Guerrini R, Jähn J, Muhle H, Auvin S, Korenke G C, Philip S, Keimer R, Striano P, Wolf N I, Püst B, Thiels Ch, Fogarasi A, Waltz S, Kurlemann G, Kovacevic-Preradovic T, Ceulemans B, Schmitt B, Philippi H, Tarquinio D, Buerki S, von StOlpnagel C, Kluger G. Retrospective evaluation of low long-term efficacy of antiepileptic drugs and ketogenic diet in 39 patients with CDKL5-related epilepsy. Eur J Paediatr Neurol. 2016; January; 20(1):147-51. 1), with an overall responder rate of 43% (3/7 subjects), with 1 additional subject nearly achieving responder status at 3 months and 33% (2/6 subjects) at 6 months. This is compared to an overall response rate less than 10% for the majority of AEDs and steroids at 6 months. Five of the 7 subjects had improvement in seizure-free days, which, in several cases, was markedly improved.

### Conclusion

Based upon known mechanism of action, preclinical and clinical data, and narrative reports from the investigators, ganaxolone has the potential to address seizure and non-seizure related problems including anxiety, poor social interaction, motor deficits and poor sleep, all of which are common and severely disabling in children with CDKL5 deficiency disorder, PCDH19-related epilepsy, and other genetic epilepsies.

The study to date in PCDH19 patients has shown that: (i) the median change in 28-day seizure frequency from baseline in the ITT (intent-to-treat) population (primary endpoint) was a decrease of 26% (n=11, 4 patients had LOCF); (ii) the median change from baseline in seizure-free days in the ITT population (key secondary endpoint) was an increase of 14% (n=11); (iii) the Clinical Global Impression Scale rated by Investigators (CGI-I) and Caregivers (CGI-P) was consistent with seizure control; (iv) two subjects completed the 52 extension and continue to receive ganaxolone through an investigator-initiated IND.

A further cohort of subjects in the study had PCDH19 epilepsy; and this cohort has completed the study. PCDH19 paediatric epilepsy is a serious and rare epileptic syndrome that predominantly affects females. The condition, which is caused by an inherited mutation of the protocadherin 19 (PCDH19) gene, located on the X chromosome, is characterised by early-onset and highly variable cluster seizures, cognitive and sensory impairment, and behavioural disturbances. The PCDH19 gene encodes a protein, protocadherin 19, which is part of a family of molecules supporting the communication between cells in the CNS. As a result of mutation, protocadherin 19 may be malformed, reduced in its functions or not produced at all. The abnormal expression of protocadherin 19 is associated with highly variable and refractory seizures, cognitive impairment and behavioural or social disorders with autistic traits. Currently, there are no approved therapies for PCDH19 paediatric epilepsy.

In total, 11 female subjects between 4 and 15 years of age with a confirmed PCDH19 genetic mutation and uncontrolled seizures despite antiepileptic pharmacotherapy were enrolled. Ganaxolone was studied as an adjunctive treatment, administered as either PO liquid suspension or capsules according to the titration schedule in Tables 7 and 8, for 26 weeks after establishing up to 12 weeks of baseline seizure frequency. Primary and secondary endpoints were the same as for the CDKL5 deficiency disorder cohort.

### Example 4

The study of Example 3 was planned to investigate whether ganaxolone provides anticonvulsant efficacy for children with uncontrolled seizures in PCDH19 Epilepsy, CDD, LGS, and CSWS epilepsy in an open-label, proof-of-concept study (due to a competing trial, no subjects with Dravet Syndrome were enrolled). This example provides additional details, results and conclusions about the study of Example 3.

After establishing baseline seizure frequency, qualifying subjects entered the study and were treated with open-label ganaxolone oral suspension or ganaxolone capsules at doses up to a maximum of 1800 mg/day for up to 6 months. Maximum study participation was 94 weeks: a screening period up to 12 weeks to establish baseline seizure frequency, up to 26 weeks of treatment, a 52 week extension for subjects who benefited from ganaxolone treatment, and up to 4 weeks of down titration period. Inclusion criteria included a PCDH19 genetic mutation or a CDD genetic mutation, confirmed by genetic testing in a certified genetic laboratory and considered to be pathogenic or likely related to the epilepsy syndrome (subjects with Dravet Syndrome would have had to have had an SCN1A mutation confirmed by genetic testing in a certified genetic laboratory and considered to be pathogenic or likely related to the epilepsy syndrome). Subjects enrolled in the CSWS cohort must have had a clinical diagnosis of CSWS determined by a child neurologist with current or historical EEG during sleep consistent with this diagnosis (e.g., continuous [85% to 100%] mainly bisynchronous 1.5 to 2 Hz [and 3 to 4 Hz] spikes and waves during non-REM sleep). Refractive cases of LGS or CSWS that had prior response to steroid or ACTH could also have been enrolled. Further, seizure criteria of the subjects was that the subject had a) uncontrolled cluster seizures (3 or more seizures over the course of 12 hours) every 6 weeks or less during baseline, or bouts of status epilepticus on intermittent basis, or b) uncontrolled non-clustered seizures (focal dyscognitive, focal convulsive, atypical absences, hemiclonic seizures, spasms, or tonic-spasm seizures) with a frequency ≥4 seizures per 28-day period during baseline, or c) had ≥4 generalized convulsive (tonic-clonic, tonic, clonic, atonic seizures) per 28 day baseline period during baseline, or d) had subclinical CSWS syndrome with or without clinical events on EEG.

Ganaxolone was provided as either oral suspension or capsules and taken with food. Grapefruit and grapefruit juice were prohibited during the study.

Ganaxolone oral suspension was administered through an oral dosing syringe by parent or legal guardian 3 times daily (TID), following the morning, noon, and evening meal or snack. Each dose was separated by a minimum of 4 hours and a maximum of 8 hours. A missed dose of ganaxolone could have been taken up to 4 hours before the next scheduled dose; otherwise, the missed dose was not to be given.

Ganaxolone capsules were administered with a glass of water or other liquid 2 times daily (BID), following the morning and evening meal or snack. Ganaxolone was provided as either an oral suspension or capsules based on the subject's weight at study entry. Ganaxolone oral suspension was administered through an oral dosing syringe TID by a parent or guardian, following the morning, midday, and evening meal or snack. Each dose was separated by a minimum of 4 hours and a maximum of 8 hours. Ganaxolone capsules were administered BID, following the morning and evening meal or snack. Each dose was separated by a minimum of 8 hours and a maximum of 12 hours. A missed dose of medication could have been taken up to 8 hours before the next dose; otherwise it was not to be given. The capsules were to be swallowed whole and not opened, crushed, or chewed.

Ganaxolone suspension contained 50 mg ganaxolone/mL, hydroxypropyl methylcellulose, polyvinyl alcohol, sodium lauryl sulfate, simethicone, methyl paraben, propyl paraben, sodium benzoate, citric acid, and sodium citrate at pH 3.8-4.2 and was sweetened with sucralose and flavored with artificial cherry. The suspension had a milky appearance and was packaged in high density polyethylene (HDPE) bottles with a child resistant closure. Ganaxolone was supplied at a concentration of 50 mg/mL (ganaxolone equivalent) in 120 mL bottles, containing 110 mL ganaxolone.

Ganaxolone capsules were provided in size 00 white/opaque gelatin capsules packaged in HDPE bottles with a foil induction seal and child resistant closure. Each capsule contained either 200 mg or 225 mg ganaxolone, and hydroxypropyl methylcellulose, sucrose, polyethylene glycol 3350, polyethylene glycol 400, sodium lauryl sulfate, sodium benzoate, citric acid anhydrous, sodium methyl paraben, microcrystalline cellulose, 30% Simethicone Emulsion, gelatin capsules, polysorbate 80, and sodium chloride.

### For Subjects>30 kg

Ganaxolone treatment was initiated at a dose of 900 mg/day in two or three doses. The dose was increased by approximately 20 to 50% at intervals of not less than 3 days and not more than 2 weeks provided the current dose was reasonably tolerated, until desired efficacy was achieved or a maximally tolerated dose (MTD) level up to a maximum of 1800 mg/day was reached. Subsequent dose adjustments were made in increments of approximately 20% to 50% with a minimum of 3 days between dose changes, unless required for safety. Any and each dose escalation above 1500 mg/day required a clinic visit scheduled 4 to 6 days after the dose increased to assess safety and tolerability. The maximum allowable dose was 1800 mg/day.

### For Subjects≤30 kg

For subjects weighing 30 kg (66 lbs) or less, dosing started at 18 mg/kg/day in two or three divided doses. The dose was then increased in approximately 20% to 50% increments at intervals of not less than 3 days and not more than 2 weeks provided the current dose was reasonably tolerated, until desired efficacy was achieved or an MTD level was reached. Subsequent dose adjustments were made in increments of approximately 20% to 50% with a minimum of 3 days between dose changes, unless required for safety. Any and each dose escalation above 54 mg/kg/day required a clinic visit scheduled 4 to 6 days after the dose increased to assess safety and tolerability. The maximum allowable dose was 63 mg/kg/day (to a maximum of 1800 mg/day).

### Efficacy Assessments

The primary outcome measure was the percentage change in seizure frequency (both individual seizures and clusters) per 28 days relative to baseline.

Secondary efficacy outcome measures included evaluation of the percent change in seizure frequency (individual seizures only) per 28-day period from baseline; percent changes in cluster frequency per 28-day period from baseline; percent change in the number of seizures per cluster; percent change in seizure frequency (individual and seizures in clusters) per 28-day period from baseline per seizure subtype; longest period of time seizure or cluster free (%); change in the number of both individual seizure and cluster free days per 28-day period from baseline; change in the number of cluster free days per 28-day period from baseline; change in the number of individual seizure free days per 28-day period from baseline; proportion of subjects with ≥25%, 50% or 75% reduction in 28-day seizure frequency (individual seizures and seizures in clusters) compared with baseline; and the Clinical Global Impression of Improvement: Clinician (CGII-C) and Clinical Global Impression of Improvement:
Patient/Caregiver (CGII-P).

Post baseline 28-day total seizure frequency was calculated as the total number of individual seizures and clusters in the 26-week open-label treatment period divided by the number of days with available seizure/cluster data in the period, multiplied by 28. Baseline 28-day total seizure frequency was calculated as the total number of individual seizures and clusters in the baseline period divided by the number of days with available seizure/cluster count data in the period, multiplied by 28. The calculation for percent change from baseline in 28-day total seizure frequency was done as follows for each subject:

$$( \frac{[ \begin{matrix}( Post - baseline 28 - day seizure frequency ) - \\ ( Baseline 28 - day seizure frequency )\end{matrix} ]}{( Baseline 28 - day seizure frequency )} ) \times 100 \%$$

The baseline and post-baseline values and the arithmetic and percent changes from baseline in 28-day total seizure frequency were summarized by cohort separately using descriptive statistics in the MITT population and PP population if they differ.

Secondary efficacy analyses were as follows: Percent change in individual seizure frequency per 28-day period from baseline; Percent change in cluster frequency per 28-day period from baseline; Percent change in the average number of seizures per cluster from baseline; Percent change in total seizure frequency (individual seizures and clusters) per 28 day period from baseline per seizure subtype; Change in percentage of individual seizure and cluster-free days from baseline; Change in percentage of individual seizure-free days from baseline; Change in percentage of cluster-free days from baseline; Change in the longest period of time individual seizure and cluster-free (%) from baseline; Proportion of subjects with 25%, 50%, or 75% reduction in 28-day total seizure frequency (sum of individual seizures and clusters) compared with baseline; and Frequency and percentage of responses to CGII-C (Clinical Global Impression of Improvement: Clinician) and CGII-P (Clinical Global Impression of Improvement: Patient/Caregiver). All secondary efficacy variables were summarized using descriptive statistics.

A total of 30 subjects were enrolled in the study: one-half (15 subjects) completed the 26-week open-label treatment period and one-half (15 subjects) discontinued the study. In total, the main reasons for study discontinuation in the safety population were lack of efficacy (8 subjects [26.7%]), and AE or SAE (4 subjects [13.3%)]). All 30 (100.0%) subjects were in the safety and MITT population. Table 13 provides a disposition of the subjects over the 26-week open-label period.

TABLE 13
Disposition of Subjects (26-week Open-label Period, All Enrolled Subjects)

| | CDKL5 | | CSWS | | LGS | PCDH19 | | Total | |
|---|---|---|---|---|---|---|---|---|---|
| | N = 7 | | N = 2 | | N = 10 | N = 11 | | N = 30 | |
| Category | n (%) | | n (%) | | n (%) | n (%) | | n (%) | |
| Subjects Enrolled | 7 | (100.0) | 2 | (100.0) | 10 (100.0) | 11 | (100.0) | 30 | (100.0) |
| Safety Population^{1,2} | 7 | (100.0) | 2 | (100.0) | 10 (100.0) | 11 | (100.0) | 30 | (100.0) |
| MITT Population^{1,3} | 7 | (100.0) | 2 | (100.0) | 10 (100.0) | 11 | (100.0) | 30 | (100.0) |
| PP Population^{1,4} | 7 | (100.0) | 2 | (100.0) | 8 (80.0) | 10 | (90.9) | 27 | (90.0) |
| Subjects in Safety | 4 | (57.1) | 0 | | 5 (50.0) | 6 | (54.5) | 15 | (50.0) |
| Population completing 26- | | | | | | | | | |
| week open-label period⁵ | | | | | | | | | |
| Subjects in the Safety | 3 | (42.9) | 2 | (100.0) | 5 (50.0) | 5 | (45.5) | 15 | (50.0) |
| Population discontinued | | | | | | | | | |
| during the 26-week open- | | | | | | | | | |
| label period⁵ | | | | | | | | | |

Reasons for discontinuation

| AE or SAE | 0 | | 1 | (50.0) | 1 (10.0) | 2 | (18.2) | 4 | (13.3) |
|---|---|---|---|---|---|---|---|---|---|
| Lack of efficacy | 1 | (14.3) | 1 | (50.0) | 3 (30.0) | 3 | (26.7) | 8 | (26.7) |

| Laboratory abnormality | 0 | | 0 | 0 | 0 | 0 | |
|---|---|---|---|---|---|---|---|
| that was not an AE/SAE | | | | | | | |
| Lost to follow up | 0 | | 0 | 0 | 0 | 0 | |
| Noncompliance with protocol | 0 | | 0 | 1 (10.0) | 0 | 1 | (3.3) |
| Other | 1 | (14.3) | 0 | 0 | 0 | 1 | (3.3) |
| Pregnancy | 0 | | 0 | 0 | 0 | 0 | |
| Withdrew consent | 1 | (14.3) | 0 | 0 | 0 | 1 | (3.3) |

AE = adverse event; CDKL5 = cyclin-dependent kinase-like 5 Deficiency Disorder (CDD); CSWS = continuous spike wave in sleep; LGS = Lennox-Gastaut Syndrome; MITT = Modified Intent-to-Treat; PCDH19 = protocadherin 19; PP = Per Protocol; SAE = serious adverse event.
¹Percentages are based on all enrolled subjects.
²The Safety Population included all subjects entered into the study who received at least 1 dose of study drug.
³The MITT Population included all subjects entered into the study who received at least 1 dose of study drug and provided at least 1 day of post-baseline calendar data.
⁴The PP Population included subjects who received study drug for at least 6 weeks, at doses between 900 mg/day and 1800 mg/day and were without a major protocol violation.
⁵Percentages are based on the Safety Population

Demographics and other baseline characteristics for the MITT and PP Populations were similar to those for the safety population.

TABLE 14
Demographic and Other Baseline Characteristics (Safety Population)

| | | | | | |
|---|---|---|---|---|---|
| | CDKL5 | CSWS | LGS | PCDH19 | Total |
| Category | N = 7 | N = 2 | N = 10 | N = 11 | N = 30 |

Age (years)

| n | 7 | | 2 | | 10 | | 11 | | 30 | |
|---|---|---|---|---|---|---|---|---|---|---|
| Mean (SD) | 7.57 | (5.167) | 11.55 | (5.728) | 9.12 | (2.352) | 9.00 | (3.956) | 8.88 | (3.834) |

| | | | | | |
|---|---|---|---|---|---|
| Median | 7.70 | 11.55 | 9.40 | 8.30 | 8.70 |
| Min, Max | 2.6, 16.5 | 7.5, 15.6 | 4.5, 13.1 | 5.0, 16.4 | 2.6, 16.5 |

Gender, n (%)

| Male | 1 | (14.3) | 1 | (50.0) | 3 | (30.0) | 0 | | 5 | (16.7) |
|---|---|---|---|---|---|---|---|---|---|---|
| Female | 6 | (85.7) | 1 | (50.0) | 7 | (70.0) | 11 | (100.0) | 25 | (83.3) |

Ethnicity, n (%)

| Hispanic or Latino | 0 | | 0 | | 4 | (40.0) | 3 | (27.3) | 7 | (23.3) |
|---|---|---|---|---|---|---|---|---|---|---|
| Non-Hispanic or Latino | 7 | (100.0) | 2 | (100.0) | 6 | (60.0) | 8 | (72.7) | 23 | (76.7) |

Race, n (%)

| American Indian or | 0 | | 0 | | 0 | | 0 | | 0 | |
|---|---|---|---|---|---|---|---|---|---|---|
| Alaska Native | | | | | | | | | | |
| Asian | 0 | | 0 | | 0 | | 0 | | 0 | |
| Black or African | 0 | | 0 | | 2 | (20.0) | 0 | | 2 | (6.7) |
| American | | | | | | | | | | |
| Native Hawaiian or | 0 | | 1 | (50.0) | 0 | | 0 | | 1 | (3.3) |
| Other Pacific Islander | | | | | | | | | | |
| White | 7 | (100.0) | 1 | (50.0) | 4 | (40.0) | 10 | (90.9) | 22 | (73.3) |
| Other | 0 | | 0 | | 3 | (30.0) | 0 | | 3 | (10.0) |
| Multiple | 0 | | 0 | | 1 | (10.0) | 1 | (9.1) | 2 | (6.7) |
| Number of Concomitant | | | | | | | | | | |
| AEDs Taken Prior to | | | | | | | | | | |
| Treatment, n (%) | | | | | | | | | | |
| 0 | 5 | (71.4) | 2 | (100.0) | 8 | (80.0) | 8 | (72.7) | 23 | (76.7) |

| | | | | | | | | |
|---|---|---|---|---|---|---|---|---|
| 1 | 1 | (14.3) | 0 | 0 | 1 | (9.1) | 2 | (6.7) |

| | | | | | | | |
|---|---|---|---|---|---|---|---|
| 2 | 0 | 0 | 1 | (10.0) | 0 | 1 | (3.3) |
| 3 | 0 | 0 | 1 | (10.0) | 0 | 1 | (3.3) |

| 5 | 0 | | 0 | 0 | 1 | (9.1) | 1 | (3.3) |
|---|---|---|---|---|---|---|---|---|
| 6 | 1 | (14.3) | 0 | 0 | 1 | (9.1) | 2 | (6.7) |

AED = anti-epilepsy drug; CDKL5 = cyclin-dependent kinase-like 5 Deficiency Disorder (CDD); CSWS = continuous spike wave in sleep; LGS = Lennox-Gastaut Syndrome; PCDH19 = protocadherin 19.

### Primary Efficacy Analysis

The percent change in 28-day total seizure frequency for the sum of individual seizures and clusters in the 26-week open-label treatment period relative to the baseline is presented for the MITT population in Table 13. Through the first 3 months (at Day 91), the mean percent change from baseline was 31.23% (SD=41.44%), 122.10% (SD=321.12%), and 52.83% (SD=234.08%) for the for the CDD, LGS, and PCDH19 cohorts, respectively. The median percent change at Day 91 was 47.34%, 10.22%, and 25.98% for the CDD, LGS, and PCDH19 cohorts, respectively.

At Week 26, the mean percent change from baseline was 20.55% (SD=60.59%), 125.38% (SD=319.05%), and 46.36% (SD=235.66%) for the CDD, LGS, and PCDH19 cohorts, respectively. The median percent change from baseline to Week 26 was 37.70%, 9.19%, and 24.59% for the CDD, LGS, and PCDH19 cohorts, respectively.

In the PP Population, the mean percent change in 28-day total seizure frequency from baseline to Week 26 was 20.55%, 18.43%, and 60.99% for the CDD, LGS, and PCDH19 cohorts, respectively. The median percent change from baseline to Week 26 was 37.70%, 11.15%, and 22.11% for the CDD, LGS, and PCDH19 cohorts, respectively

TABLE 15
Summary of 28-day Seizure Frequency for Sum of Individual
Seizures and Clusters (MITT Population)

| | | | |
|---|---|---|---|
| | CDKL5 | LGS | PCDH19 |
| Interval Statistics | N = 7 | N = 10 | N = 11 |

Baseline

| | | | | | |
|---|---|---|---|---|---|
| Mean (SD) | 205.72 | (221.601) | 111.07 (131.835) | 21.27 | (31.605) |

| | | | |
|---|---|---|---|
| Median | 104.67 | 63.49 | 12.92 |
| Min, Max | 33.7, 669.3 | 9.3, 461.0 | 2.7, 112.0 |

Post-baseline Through Month 3 (Day 91)

| | | | | | |
|---|---|---|---|---|---|
| Mean (SD) | 129.78 | (184.270) | 263.91 (502.477) | 19.82 | (30.569) |

| | | | |
|---|---|---|---|
| Median | 55.69 | 75.91 | 7.78 |
| Min, Max | 39.2, 544.4 | 6.2, 1652.0 | 0.0, 106.8 |

Percent Change from Baseline (at Day 91)

| | | | | | |
|---|---|---|---|---|---|
| Mean (SD) | −31.23 | (41.438) | 122.10 (321.124) | 52.83 | (234.084) |

| | | | |
|---|---|---|---|
| Median | −47.34 | −10.22 | −25.98 |
| Min, Max | −80.9, 36.8 | −68.1, 904.3 | −100.0, 723.2 |

Post-baseline 26-week Open-label Period

| | | | | | |
|---|---|---|---|---|---|
| Mean (SD) | 137.70 | (196.024) | 263.66 (502.176) | 19.85 | (30.850) |

| | | | |
|---|---|---|---|
| Median | 67.29 | 65.08 | 5.42 |
| Min, Max | 37.3, 579.5 | 5.6, 1652.0 | 0.0, 106.8 |

Percent Change from Baseline (at Week 26)

| | | | | | |
|---|---|---|---|---|---|
| Mean (SD) | −20.55 | (60.588) | 125.38 (319.051) | 46.36 | (235.661) |

| | | | |
|---|---|---|---|
| Median | −37.70 | −9.19 | −24.59 |
| Min, Max | −85.3, 99.9 | −71.2, 904.3 | −100.0, 723.2 |

CDKL5 = cyclin-dependent kinase-like 5 Deficiency Disorder (CDD); LGS = Lennox-Gastaut Syndrome; MITT = Modified Intent-to-Treat; PCDH19 = protocadherin 19.
Note:
Frequency of seizures included all seizure subtypes presented as individual or cluster seizures. Within each interval, 28-day seizure frequency was calculated as the total number of seizures in the interval divided by the number of days with available seizure data in the interval, multiplied by 28. The baseline interval consisted of the 12 weeks prior to the first dose. Percent changes were calculated only for subjects with non-zero Baseline values.

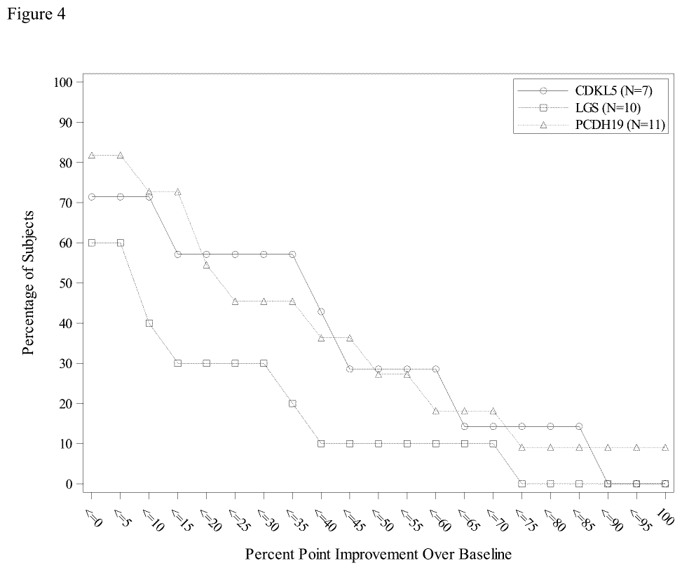

FIG. 4 presents the cumulative responder curve in terms of the 28-day seizure frequency for the sum of individual seizures and clusters.

A summary of the percent change in 28-day individual seizure frequency relative to baseline for the MITT population is presented in Table 16. The CDD and PCDH19 cohorts experienced fewer individual seizures at Day 91 compared to baseline (mean percent change from baseline of −30.33% [SD=39.83%] and −16.92% [SD=89.11%], respectively) while the LGS cohort experienced an increase in individual seizure frequency at Day 91 compared to baseline (mean percent change from baseline of 226.72% [SD=496.75%]). At Week 26, the trend remained the same with a mean percent change from baseline of −21.06 (SD=59.25%) for the CDD cohort, 229.90% (SD=494.16%) for the LGS cohort, and −23.95% (SD=88.22%) for the PCDH19 cohort.

TABLE 16
Summary of 28-day Individual Seizure Frequency (MITT Population)

| | | | |
|---|---|---|---|
| | CDKL5 | LGS | PCDH19 |
| Interval Statistics | N = 7 | N = 10 | N = 11 |

Baseline 28-day Individual Seizure Frequency

| n | 7 | | 10 | 11 | |
|---|---|---|---|---|---|
| Mean (SD) | 153.88 | (150.890) | 101.57 (126.196) | 16.61 | (32.662) |

| | | | |
|---|---|---|---|
| Median | 103.33 | 59.13 | 4.10 |
| Min, Max | 33.7, 485.9 | 0.0, 429.0 | 0.3, 112.0 |

Post-baseline Through Month 3 (Day 91)

| n | 7 | | 10 | 11 | |
|---|---|---|---|---|---|
| Mean (SD) | 103.69 | (131.580) | 256.48 (503.934) | 7.29 | (9.225) |

| | | | |
|---|---|---|---|
| Median | 53.82 | 64.71 | 2.49 |
| Min, Max | 29.6, 397.0 | 5.0, 1652.0 | 0.0, 28.9 |

Percent Change from Baseline (at Day 91)

| n | 7 | | 9 | 11 | |
|---|---|---|---|---|---|
| Mean (SD) | −30.33 | (39.827) | 226.72 (496.750) | −16.92 | (89.106) |

| | | | |
|---|---|---|---|
| Median | −46.64 | −9.56 | −38.48 |
| Min, Max | −80.9, 36.8 | −67.3, 1265.8 | −100.0, 203.7 |

Post-baseline 26-Week Open-label Period

| n | 7 | | 10 | 11 | |
|---|---|---|---|---|---|
| Mean (SD) | 103.70 | (118.977) | 257.07 (503.453) | 7.28 | (9.833) |

| | | | |
|---|---|---|---|
| Median | 67.29 | 55.93 | 2.26 |
| Min, Max | 23.5, 367.4 | 5.0, 1652.0 | 0.0, 29.9 |

Percent Change from Baseline (at Wek 26)

| n | 7 | | 9 | 11 | |
|---|---|---|---|---|---|
| Mean (SD) | −21.06 | (59.247) | 229.90 (494.164) | −23.95 | (88.222) |

| | | | |
|---|---|---|---|
| Median | −38.10 | 5.04 | −34.48 |
| Min, Max | −84.8, 99.9 | −72.8, 1265.8 | −100.0, 203.7 |

CDKL5 = Cyclin-dependent kinase-like 5 Deficiency Disorder (CDD); LGS = Lennox-Gastaut Syndrome; MITT = Modified Intent-to-Treat; PCDH19 = protocadherin 19.
Note:
Frequency of seizures included all seizure subtypes presented as individual seizures. Baseline 28-day seizure frequency was calculated as the total number of seizures in the baseline period (4 weeks to 12 weeks retrospective baseline) divided by the number of days with available seizure data in the period, multiplied by 28. Post-baseline 28-day seizure frequency was calculated as the total number of seizures in the 26-week open-label period divided by the number of days with available seizure data in the period, multiplied by 28.

With respect to the clinical global impression of improvement, at the end of Week 26 in the CDD cohort, 3 subjects (42.9%) were much improved and 0 subjects were very much improved; in the LGS cohort, 1 subject (14.3%) was much improved and 1 subject (14.3%) was very much improved; and in the PCDH19 cohort, 2 subjects (22.2%) were much improved and 2 subjects (22.2%) were very much improved.

Ganaxolone was generally safe and well-tolerated in subjects with epilepsy disorders. Overall, based on evaluation of treatment-emergent adverse events (“TEAEs”) in the safety population, treatment with ganaxolone was well tolerated across cohorts. In the CDD cohort, 6 subjects (85.7%) experienced a total of 45 TEAEs; in the CSWS cohort, 1 subject (50.0%) had 7 TEAEs; in the LGS cohort, 7 subjects (70.0%) had 24 TEAEs; and in the PCDH19 cohort, 11 subjects (100.0%) had 95 TEAEs.

A total of 83.3% of subjects overall experienced TEAEs: 23.3% mild, 46.7% moderate, 13.3% severe. Six subjects (20.0%) experienced SAEs, 16 subjects (53.3%) had treatment-related TEAEs, and 4 subjects (13.3%) had a TEAE that led to discontinuation of study drug. No deaths were reported in this study.

Preliminary findings regarding the correlation of baseline endogenous allopregnanolone levels and seizure frequency change (efficacy) are presented in FIG. 10 which is a plot of plasma allopregnanolone in each subject compared to the percentage change in seizure frequency with the administration of ganaxolone in accordance with this Example. In FIG. 10, each closed circle represents a unique subject in the trial. In FIG. 10, a percentage change in seizure frequency of −100% means complete freedom from seizure activity, i.e., that the subject had not experienced any seizures during the 26 week period of the study. That would represent the best possible result. Anywhere between 0 and −100% shows efficacy for the ganaxolone dosing regimen of this Example. As can be seen from the results set forth in FIG. 10, subjects who had a plasma allopregnanolone level of less than 200 μg/ml (and less than 100 μg/ml, and less than about 75 μg/ml, and in certain patients less than about 50 μg/ml) responded best to the ganaxolone dosing regimen of this Example.

### Example 5

### Single/Dose Fast Fed Study

When the 0.3 micron ganaxolone suspension of Example 1 was administered to healthy volunteers at 200 mg fasted and 400 mg in the fasted and high-fat state study. A fed/fasted effect of 2 and 3-fold was seen on AUC_{(0-∞)} and Cₘₐₓ, respectively. The 200 and 400 mg dose in the fasted state were dose proportional.

Summary of ganaxolone pharmacokinetic parameters following a single dose of 0.3 micron ganaxolone suspension in healthy volunteers in the fed and fasted state is provided in Table 17:

TABLE 17

| | Ganaxolone Dose and Condition | | | | | |
|---|---|---|---|---|---|---|
| | 200 mg Fasted | | 400 mg Fasted | | 400 mg Fed | |
| Parameter | (N = 6) | | (N = 6) | | (N = 6) | |
| AUC₍₀₋₂₄₎ (ng · hr/mL) | 184.3 | (104.52) | 298.1 | (144.89) | 924.9 | (394.17) |
| AUC_{(0-∞)} (ng · hr/mL) | 327.1 | (89.16) | 540.7 | (177.25) | 1169.5 | (432.82) |
| Cₘₐₓ (ng/mL) | 37.27 | (25.374) | 57.27 | (37.651) | 166.31 | (60.976) |
| Cₘₐₓ/Dose (ng/mL/mg) | 0.1864 | (0.12687) | 0.1432 | (0.09413) | 0.4158 | (0.15244) |
| Tₘₐₓ (hr) | 1.000 | (1.00, 1.50) | 1.00 | (0.50, 1.02) | 1.250 | (0.50, 2.00) |
| T_{1/2} (hr) | 22.32 | (24.203)^{a} | 21.45 | (18.897) | 29.27 | (0.842) |

AUC = area under the concentration time curve; Cₘₐₓ = maximum concentration; T_{1/2} = half-life; Tₘₐₓ = time of maximum concentration
^{a}N = 3

The 0.3 micron ganaxolone capsule of Example 2 was tested at single fed/fasted doses of 200, 400 and 600 mg as well as at multiple doses of 200, 400 and 600 mg BID (400 mg/day, 800 mg/day and 1200 mg/day) in healthy volunteers.

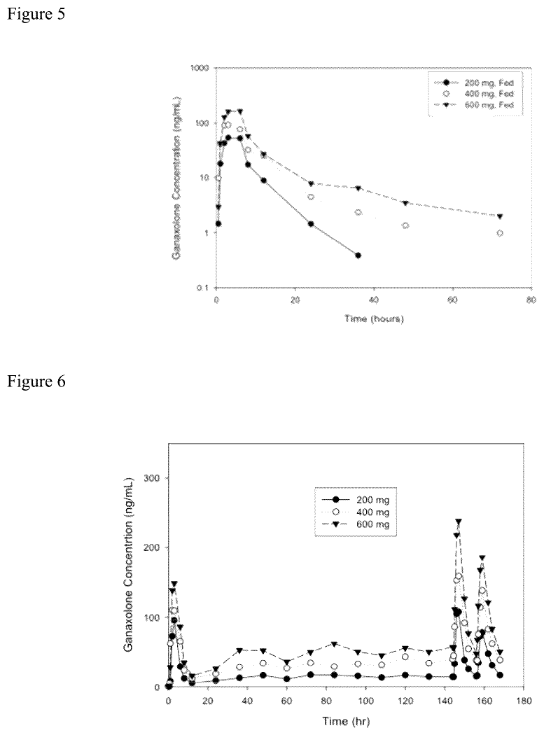

Mean ganaxolone plasma concentration profile following a single oral dose of ganaxolone 0.3 micron capsules of Example 2 in healthy volunteers after a high fat meal is depicted in FIG. 5.

Mean ganaxolone plasma concentration-time profiles following single and multiple BID oral doses of 0.3 micron ganaxolone capsules of Example 2 with a standard meal or snack in healthy volunteers is depicted in FIG. 6.

After PO administration, the 0.3 micron ganaxolone capsules demonstrated a rapid distribution phase followed by a longer elimination phase (FIG. 5).

Single doses in the fasted and high-fat fed state showed a fed/fasted geometric mean ratio (GMR) of 2.2, 3.2 and 4.9 for Cₘₐₓ and 1.8, 2.4 and 3.8 for AUC_{(0-∞)} for the 200, 400 and 600 mg doses, respectively. In the fed state, the AUC_{(0-∞)} and Cₘₐₓ values were close to dose proportional. In the fasted state, Cₘₐₓ and AUC_{(0-∞)} values were less than dose proportional across the 200 mg, 400 mg and 600 mg dose range, with Cₘₐₓ less proportional than AUC_{(0-∞)}. In the fed state AUC₍₀₋ₜ₎ values with 0.3 micron ganaxolone capsules at doses of 200, 400 and 600 mg were close to dose proportional (GMR of 108% and 130%, respectively) as were Cₘₐₓ values (GMR of 91% and 106%, respectively). In the fasted state, high CL of ganaxolone was observed. In the 0.3 micron capsule study, oral clearances CL/F values were not statistically different from doses of 200 to 600 mg and ranged from 586 to 433 L/h. Fasted and high fat fed PK parameters for the study are presented in FIG. 5 and FIG. 6, respectively.

Summary of ganaxolone pharmacokinetic parameters following a single oral dose of ganaxolone 0.3 micron capsules in healthy volunteers in the fasted state is provided in Table 18:

TABLE 18

| | 200 mg | | | 400 mg | | | 600 mg | | |
|---|---|---|---|---|---|---|---|---|---|
| | (N = 6) | | | (N = 6) | | | (N = 6) | | |
| Parameter | Mean | SD | CV % | Mean | SD | CV % | Mean | SD | CV % |
| Cₘₐₓ (ng/mL) | 27.9 | 14.5 | 52 | 35.7 | 18.1 | 51 | 41.0 | 21.5 | 53 |
| Tₘₐₓ^{a} (hr) | 2.00 | [1.00, 2.00] | 31 | 1.50 | [1.00, 2.00] | 37 | 2.00 | [1.00, 3.00] | 45 |
| AUC₍₀₋₂₄₎ | 164 | 45.0 | 27 | 282 | 147 | 52 | 292 | 209 | 71 |
| (ng · h/mL) | | | | | | | | | |
| AUC_{(0-∞)} | 229 | 111 | 48 | 404 | 230 | 57 | 498 | 326 | 65 |
| (ng · h/mL) | | | | | | | | | |
| CL/F (L/h) | 1040 | 430 | 41 | 1310 | 701 | 54 | 2220 | 2600 | 117 |
| T_{1/2}^{b} (hr) | 6.93 | 7.80 | 112 | 13.3 | 8.04 | 61 | 10.6 | 30.0 | 283 |

AUC = area under the concentration time curve;
CL/F = oral clearance;
Cₘₐₓ = maximum concentration;
CV % = percent coefficient of variation;
N = number of subjects;
SD = standard deviation;
T_{1/2} = half-life;
Tₘₐₓ = time of maximum concentration
^{a}Expressed as median and range.
^{b}Expressed as harmonic mean and pseudo-standard deviation based on jackknife variance.

Summary of ganaxolone pharmacokinetic parameters following a single oral dose of ganaxolone 0.3 micron capsules in healthy volunteers after a high-fat meal is provided in Table 19:

TABLE 19

| | 200 mg | | | 400 mg | | | 600 mg | | |
|---|---|---|---|---|---|---|---|---|---|
| | (N = 6) | | | (N = 6) | | | (N = 6) | | |
| Parameter | Mean | SD | CV % | Mean | SD | CV % | Mean | SD | CV % |
| Cₘₐₓ | 61.9 | 27.7 | 45 | 106 | 35.0 | 33 | 190 | 62.6 | 33 |
| (ng/mL) | | | | | | | | | |
| Tₘₐₓ^{a} (hr) | 3.00 | [2.00, 6.00] | 45 | 3.00 | [2.00, 6.00] | 51 | 4.50 | [3.00, 6.00] | 37 |
| AUC₍₀₋₂₄₎ | 419 | 202 | 48 | 815 | 396 | 49 | 1310 | 487 | 37 |
| (ng · h/mL) | | | | | | | | | |
| AUC_{(0-∞)} | 432 | 227 | 52 | 966 | 590 | 61 | 1610 | 617 | 38 |
| (ng · h/mL) | | | | | | | | | |
| CL/F (L/h) | 586 | 308 | 53 | 545 | 271 | 50 | 433 | 209 | 48 |
| T_{1/2}^{b} (hr) | 3.46 | 2.26 | 65 | 6.56 | 6.11 | 93 | 18.7 | 17.4 | 93 |

AUC = area under the concentration time curve;
CL/F = oral clearance;
Cₘₐₓ = maximum concentration;
CV % = percent coefficient of variation;
N = number of subjects;
SD = standard deviation;
T_{1/2} = half-life;
Tₘₐₓ = time of maximum concentration
^{a}Expressed as median and range.
^{b}Expressed as harmonic mean and pseudo-standard deviation based on jackknife variance.

The 0.3 micron ganaxolone capsule formulation was designed to maximise contact time in the stomach and small intestine to provide an increased effective T_{1/2} when given under repeated-dose conditions BID. With an acute dose, the capsules gave more variable PK compared to the 0.3-micron suspension presumably due to retention of particles in the stomach and small intestine resulting in more variable data 24 hours after dosing. The intra-subject variability in plasma concentrations at 24 to 72 hours post dose resulted in the elimination T_{1/2} values having the highest variability. The increase in T_{1/2} from the 200 mg to 600 mg doses of ganaxolone capsules did not appear to be a saturation effect, as the AUC values from 200 mg to 600 mg were close to dose proportional while the T_{1/2} values were 3.46 hours and 18.7 hours, respectively. The T_{1/2} increases with dose are likely attributed to the fact that at higher doses the elimination phase for this formulation was more discernible in subjects due to higher drug loading into lipophilic tissues.

GI site-specific absorption analysis on ganaxolone has not been conducted; however, almost no time of maximum concentration (Tₘₐₓ) values were in the range of anticipated delivery to the colon (7 to 10 hours), which suggests that the majority of ganaxolone absorption likely occurs in the small intestine.

Summary of ganaxolone pharmacokinetic parameters (mean [SD]) following single-doses of ganaxolone 0.3 micron formulations in healthy volunteers after a high fat meal is provided in Table 20

TABLE 20

| | | | | | |
|---|---|---|---|---|---|
| | | AUC_{(0-∞)} | Cₘₐₓ | Tₘₐₓ^{b} | T_{1/2} |
| Dose^{a} (mg) | N | (ng · hr/mL) | (ng/mL) | (hrs) | (hrs) |

(0.3-micron suspension)

| | | | | | | | | |
|---|---|---|---|---|---|---|---|---|
| 400 mg (50 mg/mL) | 6 | 1169 | (433) | 166 | (61) | 1 | (0.5-2) | 29.3 (0.84) |

(0.3-micron capsules)

| | | | | | | | | |
|---|---|---|---|---|---|---|---|---|
| 200 mg; 1 capsule | 6 | 432 | (227) | 61.9 | (27.7) | 3.0 | (2-6) | 3.5 (2.3) |
| 400 mg; 2 capsules | 6 | 966 | (590) | 106 | (35) | 3.0 | (2-6) | 6.6 (6.1) |
| 600 mg, 3 capsules | 6 | 1610 | (617) | 190 | (62.6) | 4.5 | (3-6) | 18.7 (17.4) |

AUC = area under the concentration time curve; Cₘₐₓ = maximum concentration; N = number of subjects; SD = standard deviation; T_{1/2} = half-life; Tₘₐₓ = time of maximum concentration
^{a}Concentration of dosing solution in parentheses.
^{b}Median (range)

### Example 6

### Multiple Dose PK Study

Multiple-dose studies of oral ganaxolone formulations were conducted in healthy volunteers. The 0.3 micron ganaxolone capsules were administered BID with a standard meal or snack for 7 days and at increasing doses. The PK data for these studies are presented in Table 21.

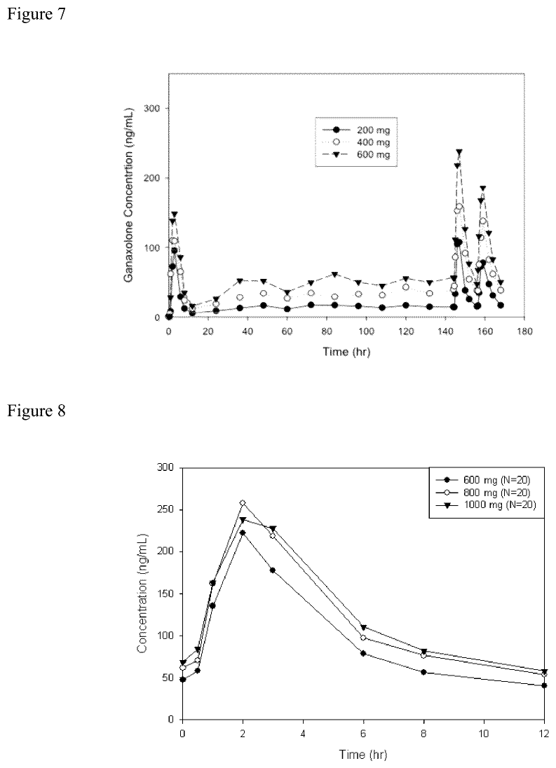

In the 7-day study with the 0.3 micron ganaxolone capsules at doses of 200, 400 and 600 mg BID, steady state was achieved within 48 hours when dosed with a standard meal or snack. At steady state, mean Cₘₐₓ and AUC₍₀₋₁₂₎ were close to dose proportional. Cₘₐₓ and AUC₍₀₋₁₂₎ were similar across doses when comparing dosing with a high-fat or standard meal/snack, with the 600 mg dose having a mean AUC₍₀₋₁₂₎ approximately 25% lower with a standard meal/snack than with a high-fat meal. Trough levels after 7 days of dosing at 200, 400 and 600 mg BID were 14.3 ng/mL, 39.4 and 56.4 ng/mL. Accumulation for AUC₍₀₋₁₂₎ was approximately 43 to 81%, yielding an effective T, with BID dosing of 7 to 10 hours. Time-dependent plasma concentration curves are shown in FIG. 7. Steady state PK of the 0.3 micron ganaxolone capsules did not demonstrate a significant diurnal effect.

Steady-state was reached within 3 days of administration of 600, 800 and 1000 mg BID ganaxolone to healthy subjects. The medium and high dose regimens were started after 3 days on the low dose and medium dose regimens, respectively. In general, ganaxolone was rapidly absorbed following PO administration and the mean Cₘₐₓ was attained within 2 hours after multiple dosing; median Tₘₐₓ was independent of dose level. Mean Cₘₐₓ was 224, 263 and 262 ng/mL for the 3 dose levels; it was statistically not dose proportional, with disproportionality driven mainly by the lack of increase in exposure from 800 mg to 1000 mg. Cₘᵢₙ, C_{avg} and AUC_{τ} showed similar trends in sub-proportionality. Proportionality was conserved from 600 mg to 800 mg. The time dependent plasma curves are shown in FIG. 8, and daily trough levels are shown in FIG. 9. The mean apparent total body CL and mean fluctuation at steady-state ranged from 609 to 770 L/hr and from 172% to 191%, respectively, over the dose range of 600 to 1000 mg ganaxolone BID. These data suggest that over the dose range of 600 to 1000 mg BID under fed conditions, exposure to ganaxolone increases though less than proportionally with increases in dose, with this disproportionality being more pronounced at the high end of the dose range.

TABLE 21
Summary of mean (SD) ganaxolone pharmacokinetic parameters following multiple
dosing of 0.3 micron ganaxolone capsules in healthy volunteers

| | | | | | |
|---|---|---|---|---|---|
| | | AUC_{(0-Last)} | Cₘₐₓ | C_{min ss} | Tₘₐₓ^{a} |
| Dose | Study Day | (ng · hr/mL) | (ng/mL) | (ng/mL) | (hrs) |

Study (BID for 7 days, N = 6) 0.3 micron capsules with a standard meal or snack

| 200 mg AM | Day 1 | 387 | (186) | 96.9 | (57.5) | NA | | 3.0 | (2-3) |
|---|---|---|---|---|---|---|---|---|---|
| Dose | | | | | | | | | |
| 200 mg AM | Day 7 | 555 | (230) | 110 | (42.7) | 14.3 | (6.5) | 2.5 | (2-3) |
| Dose | | | | | | | | | |
| 400 mg AM | Day 1 | 631 | (334) | 116 | (53.7) | NA | | 2.5 | (1-6) |
| dose | | | | | | | | | |
| 400 mg AM | Day 7 | 1030 | (449) | 169 | (77.7) | 39.4 | (20.7) | 3.0 | (2-6) |
| Dose | | | | | | | | | |
| 600 mg AM | Day 1 | 806 | (232) | 153 | (44.4) | NA | | 3.0 | (2-3) |
| Dose | | | | | | | | | |
| 600 mg AM | Day 7 | 1460 | (434) | 239 | (64.3) | 56.4 | (22.7) | 3.0 | (1-3) |
| Dose | | | | | | | | | |

Study (BID for 3 days, N = 22) 0.3 micron capsules with a standard meal or snack

| | | | | | | | | | |
|---|---|---|---|---|---|---|---|---|---|
| 1200 mg/day, | Day 6 | 1160 | (461) | 224 | (100) | 38.9 | (16.9) | 2.00 | (1-3) |
| BID | | | | | | | | | |
| 1600 mg/day, | Day 9 | 1450 | (504) | 263 | (99.2) | 52.0 | (27.3) | 2.00 | (2-3) |
| BID | | | | | | | | | |
| 2000 mg/day, | Day 12 | 1510 | (640) | 262 | (90.8) | 56.9 | (28.8) | 2.00 | (1-3) |
| BID | | | | | | | | | |

AUC = area under the concentration time curve; BID = 2 times per day; Cₘₐₓ = maximum concentration; C_{min ss} = minimum concentration at steady state; NA = Not applicable; SD = standard deviation. Tₘₐₓ = time of maximum concentration.
^{a}Median (range)
Values at Day 6.5, 9.5 and 12.5 are from evening samples collected 12 hrs after the last dose on PK sampling days.
Subjects received 600 mg ganaxolone BID on Days 4-6; 800 mg ganaxolone BID on Days 7-9; and 1000 mg ganaxolone BID on Days 10-12.

Values at Day 6.5, 9.5 and 12.5 are from evening samples collected 12 hrs after the last dose on PK sampling days.
Subjects received 600 mg ganaxolone BID on Days 4-6; 800 mg ganaxolone BID on Days 7-9; and 1000 mg ganaxolone BID on Days 10-12.

### Example 7

### Mass Balance

Mass balance has been assessed following a single PO dose of 300 mg ¹⁴C-GNX (with HP-β-CD) administered to healthy male volunteers. The total plasma radioactivity concentrations achieved were much higher than GNX plasma levels in clinical studies with non-labelled GNX. These results suggest the presence of metabolite(s) in the plasma. In addition, total radioactivity appeared to have a longer elimination half-life than intact GNX (230 hours vs ˜25 hours). Greater than 94% of total radioactivity was eliminated and collected in urine and faeces over 30 days, which indicated nearly complete recovery of all the ¹⁴C-GNX dosed. Approximately 80% of the total radioactivity was excreted in faeces and urine by Day 14. The majority of the recovered radioactivity was in the faeces (68.95%), with the remainder in the urine (25.34%). PK parameters (mean±SD) of ¹⁴C-GNX-derived total radioactivity in Healthy Male Volunteers (Study No. CA042 9402.01 [n=5]) are summarized in Table 22.

TABLE 22

| | |
|---|---|
| AUC_{(0-∞)} (μg-equiv · hr/mL) | 542.1 ± 90.4 |
| Cₘₐₓ (μg-equiv/mL) | 6.66 ± 0.91 |
| Tₘₐₓ (hrs)^{a} | 5.0 (1.5-5.0) |
| T_{1/2} (hrs) | 231.0 ± 43.0 |
| Cumulative Elimination (% Dose) | 94.30 ± 3.60 |

AUC = area under the concentration time curve; Cₘₐₓ = maximum concentration; PK = pharmacokinetics; Tₘₐₓ = time of maximum concentration; T_{1/2} = half-life
^{a}Median (range)

### Example 8

### Food Effect

Current and historical ganaxolone formulations have all demonstrated higher levels and exposure in the fed versus fasted state.

The magnitude of the fed/fasted effect with the current formulations was reduced by approximately 3-fold for Cₘₐₓ and 7- to 8-fold for AUC_{(0-∞)} when compared with the historical ganaxolone βCD Complex Suspension.

A fed/fasted study with 0.3-micron ganaxolone capsules in healthy volunteers at doses of 200, 400 and 600 mg showed an increase in the food effect with increasing doses (Table 31).

Geometric mean ratio (fed/fasted) of ganaxolone pharmacokinetic parameters following administration of 0.3-micron ganaxolone capsules to healthy volunteers are depicted in Table 23.

TABLE 23

| | GMR High-fat Fed/Fasted Ratio | |
|---|---|---|
| Dose (mg) | Cₘₐₓ | AUC_{(0-∞)} |
| 200 | 2.2 | 1.8 |
| 400 | 3.2 | 2.4 |
| 600 | 4.9 | 3.8 |

AUC = area under the concentration time curve; Cₘₐₓ = maximum concentration; GMR = geometric mean ratio.

The effect of different types of food on 0.3 ganaxolone capsules has been indirectly measured where the ratios of Cₘₐₓ and AUC_{(0-∞)} after a high-fat or standard meal were similar, as shown in Table. Another study, using a 400-mg BID dosing regimen at steady state with a standard meal versus a liquid meal (8 oz. ENSURE®, nutritional supplement), demonstrated a 1.2 fold and 1.3 ratio for Cₘₐₓ and AUC₍₀₋₁₂₎, respectively.

Mean Ratio (High-Fat/Standard Meal) of ganaxolone Pharmacokinetic Parameters Following Administration of 0.3-micron ganaxolone capsules to Healthy Volunteers are summarized in Table 24.

TABLE 24

| | High-Fat/Standard Meal Ratio | |
|---|---|---|
| Dose (mg) | Cₘₐₓ | AUC_{(0-∞)} |
| 200 mg | 0.63 | 1.01 |
| 400 mg | 0.91 | 0.94 |
| 600 mg | 1.24 | 1.17 |

AUC = area under the concentration time curve; Cₘₐₓ = maximum concentration

These results indicate that absorption of ganaxolone is enhanced in the presence of food with the 0.3-micron formulations, showing a reduced high fat to standard meal or fasted ratio as compared to previous formulations. The fed/fasted ratio also increases with increasing doses.

### Example 9

### Gender Effect

Repeated studies using healthy volunteers have not shown a gender effect for PK parameters with ganaxolone dosing. A representative example following dosing with ganaxolone β-CD suspension is shown in Table 25.

TABLE 25
Effect of gender on ganaxolone mean (SD) pharmacokinetic parameters following
a single oral dose with a high fat meal in healthy volunteers

| | Ganaxolone Dose (mg) and Gender | | | |
|---|---|---|---|---|
| | Males (n = 8) | | Females (n = 9) | |
| Parameter | 300 | 900 | 300 | 900 |
| AUC_{(0-∞)} (ng · hr/mL) | 848.7 ± 279.0 | 2387.3 ± 538.1 | 904.2 ± 220.3 | 2541.2 ± 760.5 |
| Cₘₐₓ (ng/mL) | 131.1 ± 42.8 | 310.1 ± 83.1 | 120.6 ± 28.8 | 282.3 ± 52.3 |
| Tₘₐₓ^{a} (hrs) | 2.8 (1.5-5.0) | 2.5 (1.0-5.0) | 2.0 (1.5-5.0) | 4.5 (2.0-5.0) |
| T_{1/2} (hrs) | 28.7 ± 10.7 | 35.0 ± 11.6 | 46.0 ± 21.9 | 36.0 ± 10.9 |

AUC = area under the concentration time curve; Cₘₐₓ = maximum concentration; n = number of subjects; SD = standard deviation; T_{1/2} = half-life; Tₘₐₓ = time of maximum concentration
^{a}Median (range)

### Example 10

### Biomarker

In addition, an important retrospective review of baseline endogenous neurosteroid levels in the studies described in Examples 3 and 4 revealed preliminary evidence of a strong predictive biomarkers (allopregnanolone-sulfate; Allo-S) and allopregnanolone (Allo) that may be used to identify a patient population that potentially has a much higher response rate to ganaxolone treatment than those that are biomarker-negative. It is hypothesized that this sulfated version of allopregnanolone is more readily found in circulation and may qualitatively represent allopregnanolone levels in the brain.

Methods: Individuals (n=11) with a confirmed PCDH19 mutation and minimum seizure burden were enrolled between May 2015 and November 2015 at six centers in the U.S. and Italy. Seizure frequency change (%) was assessed as the primary endpoint and a responder was defined as having a 25% or greater decrease in seizure rate. Plasma neurosteroid levels were quantified using a previously published GC/MS method (doi:10.1016/S0028-3908(99)00149-5). In two cases, baseline neurosteroid levels were not measured. In these cases, the values from 6 months were used as neurosteroid levels were observed not to change significantly over time.

Results: The median change in 28-day seizure frequency (all seizure types) from baseline for all-comers (n=11) was a decrease of 26%. In this group, average plasma allopregnanolone-sulfate (Allo-S) concentration was 4,741 pg mL⁻¹ (median=433 pg mL⁻¹). The responder analysis and correlation with Allo-S demonstrated two discrete populations. Responders (n=6) (≥25% decrease in seizure rate) and non-responders (n=5) had plasma Allo-S concentrations of 501±430 pg mL⁻¹ and 9,829±6,638 pg mL⁻¹, respectively (mean±SD, p=0.05, Mann-Whitney) (FIG. 10).

The biomarker-positive group significantly improved (p=0.02, Wilcoxon) whereas the biomarker-negative (high Allo-S) group did not improve, but also did not significantly deteriorate (p=0.25, Wilcoxon), when comparing seizure frequency at 6 months to baseline.

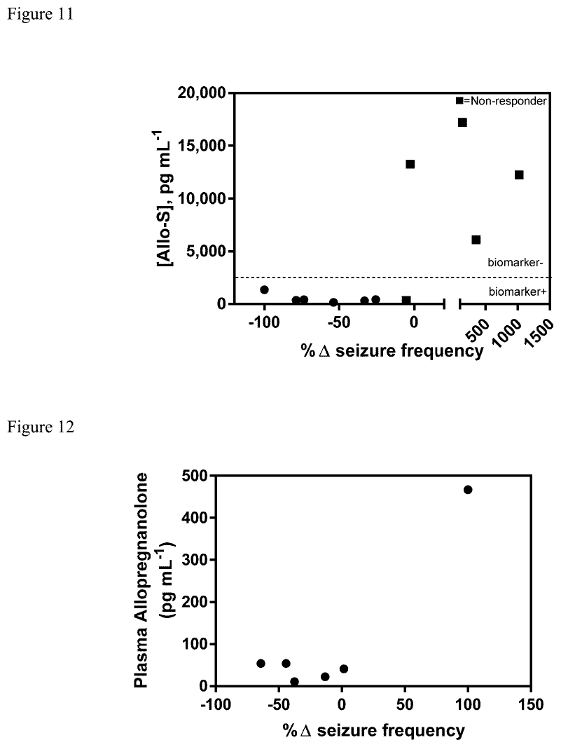

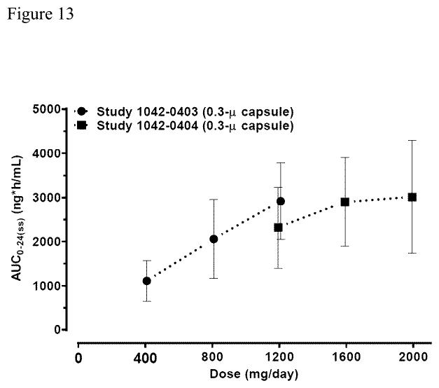

Retrospective analysis of biomarker-positive (n=7, Allo-S<2,500 pg mL⁻¹) versus biomarker-negative (n=4, Allo-S>2,500 pg mL⁻¹) subjects yielded median % change seizure rates of −53.9% and 247%, respectively (p=0.006, Mann-Whitney). (FIG. 11). Further, the biomarker-positive group significantly improved (p=0.02, Wilcoxon Signed Rank) whereas the biomarker-negative group did not significantly deteriorate (p=0.25, Wilcoxon Signed Rank) when comparing seizure frequency at 6 months to baseline. FIG. 11 % shows change seizure frequency (primary efficacy endpoint) stratified by biomarker+ and biomarker− subjects.

Allopregnanolone can be used as a biomarker in subjects with CDKL5. FIG. 12 shows % change in seizure frequency in responders in the CDKL5 cohort. Each closed circle represents a unique subject in the trial. “−100 change” means complete seizure freedom, patient not experiencing any seizures during that 26 week period. Anywhere between “0” and “−100%” is showing efficacy. The patient with increase—had a worsening of the seizures during the study. That patient had about 10× level of allopregnanolone as other patients that had positive (reduced seizure) effect.

These results indicate, e.g., that a plasma neurosteroid (allopregnanolone-sulfate (Allo-S) and/or allopregnanolone (Allo)) biomarker that may be used to predict seizure response when treated with ganaxolone, e.g., in PCDH19, CDD, and other epileptic encephalopathies.

### REFERENCES

American Academy of Neurology (Practice Guideline Summary: Sudden Unexpected Death in Epilepsy Incidence Rates and Risk Factors April, 2017
Archer H L, Evans J, Edwards S et al. CDKL5 mutations cause infantile spasms, early onset seizures, and severe mental retardation in female patients. J Med Genet 2006; 43: 729-734.
Bahi-Buisson N, Nectoux J, Rosas-Vargas H, Milh M, Boddaert N, Girard B, Cances C, Ville D, Afenjar A, Rio M, Hron D, N'guyen Morel M A, Arzimanoglou A, Philippe C, Jonveaux P, Chelly J, Bienvenu T. Key clinical features to identify girls with CDKL5 mutations. Brain. 2008a. 131:2647-2661
Bahi-Buisson N, Kaminska A, Boddaert N, Rio M, Afenjar A, Grard M, Giuliano F, Motte J, Hron D, Morel M A, Plouin P, Richelme C, des Portes V, Dulac O, Philippe C, Chiron C, Nabbout R, Bienvenu T. The three stages of epilepsy in patients with CDKL5 mutations. Epilepsia. 2008b 49:1027-1037.
Barbiero I, Peroni D, Tramarin M, Chandola C, Rusconi L, Landsberger N, Kilstrup-Nielsen C. The neurosteroid pregnenolone reverts microtubule derangement induced by the loss of a functional CDKL5-IQGAP1 complex. Hum Mol Genet. 2017 Jun. 21. doi: 10.1093/hmg/ddx237. [Epub ahead of print] ClinicalTrials.gov Identifier: NCT02358538
Elia M, Falco M, Ferri R, Spalletta A, Bottitta M, Calabrese G, Carotenuto M, Musumeci S A, Lo Giudice M, Fichera M. CDKL5 mutations in boys with severe encephalopathy and early-onset intractable epilepsy. Neurology. 2008; 71:997-999.
Fehr S, Wilson M, Downs J, Williams S, Murgia A, Santori S, Vecchi M, Ho G, Polli R, Psoni S, Bao X, de Klerk N, Leonard H, Christodoulou J. The CDKL5 Deficiency Disorder is an independent clinical entity associated with early-onset encephalopathy. Eur J Hum Genetics 2013; 21; 266-273.
Guerrini R, Parrini E. Epilepsy in Rett syndrome, and CDKL5- and FOXG1-gene-related encephalopathies. Epilepsia. 2012; 53:2067-78.
Hagebeuk E E O, Van den Bossche R A S, De Weerd A W. Respiratory and sleep disorders in female children with atypical Rett syndrome caused by mutations in the CDKL5 gene. Dev Med Child Neurol. 2012; 55:480-4
Kalscheuer V M, Tao J, Donnelly A, Hollway G, Schwinger E, Kbart S, Menzel C, Hoeltzenbein M, Tommerup N, Eyre H, Harbord M, Haan E, Sutherland G R, Ropers H H, Gcz J. Disruption of the serine/threonine kinase 9 gene causes severe X-linked infantile spasms and mental retardation. Am J Hum Genet. 2003; 72:1401-1411.
Breakthrough Therapy Designation Request Marinus Pharmaceuticals, Inc. Ganaxolone for the Treatment of CDKL5 23 Aug. 2017
Kilstrup-Nielsen C, Rusconi L, La Montanara P, Ciceri D, Bergo A, Bedogni F, Landsberger N. Review Article: What we know and would like to know about CDKL5 and its involvement in Epileptic Encephalopathy. Neural Plast. 2012; article ID: 728267.
Lappalainen J, Tsai J, Amerine W, Patroneva. A Multicenter, Double-Blind, Randomized, Placebo-Controlled Phase 3 Trial to Determine the Efficacy and Safety of Ganaxolone as Adjunctive Therapy for Adults with Drug-Resistant Focal-Onset Seizures Neurology 2017: 88, 16 Supplement P5.237.
Loulou Foundation [Internet].
Mangatt M, Wong K, Anderson B, Epstein A, Hodgetts S, Leonard H. Downs J. Prevalence and onset of comorbidities in the CDKL5 Deficiency Disorder differ from Rett syndrome. Orphanet Journal of Rare Diseases. 2016; 11:39
Mari F, Azimonti S, Bertani I, Bolognese F, Colombo E, Caselli R, Scala E, Longo I, Grosso S, Pescucci C, Ariani F, Hayek G, Balestri P, Bergo A, Badaracco G, Zappella M, Broccoli V, Renieri A, Kilstrup-Nielsen C, Landsberger N. CDKL5 belongs to the same molecular pathway of MeCP2 and it is responsible for the early-onset seizure variant of Rett syndrome. Hum Mol Genet. 2005; 14:1935-1946.
Mei D, Marini C, Novara F et al: Xp22.3 genomic deletions involving the CDKL5 gene in girls with early onset epileptic encephalopathy. Epilepsia. 2010; 51: 647-654.
Melani F, Mei D, Pisano T et al: CDKL5 gene-related epileptic encephalopathy: electroclinical findings in the first year of life. Dev Med Child Neurol. 2011; 53: 354-360.
Mori Y, Downs J 1, Wong K, Anderson B, Epstein A, Leonard H. Impacts of caring for a child with the CDKL5 Deficiency Disorder on parental wellbeing and family quality of life. Orphanet J Rare Dis. 2017 Jan. 19; 12(1):16. doi: 10.1186/s13023-016-0563-3.
Müller A, Helbig I, Jansen C, Bast T, Guerrini R, Jähn J, Muhle H, Auvin S, Korenke G C, Philip S, Keimer R, Striano P, Wolf N I, Püst B, Thiels Ch, Fogarasi A, Waltz S, Kurlemann G, Kovacevic-Preradovic T, Ceulemans B, Schmitt B, Philippi H, Tarquinio D, Buerki S, von Stülpnagel C, Kluger G. Retrospective evaluation of low long-term efficacy of antiepileptic drugs and ketogenic diet in 39 patients with CDKL5-related epilepsy. Eur J Paediatr Neurol. 2016; January; 20(1):147-51. 1
Nemos C, Lambert L, Giuliano F et al, Mutational spectrum of CDKL5 in early-onset encephalopathies: a study of a large collection of French patients and review of the literature. Clinical Genetics. 2009; 76 (4), 357-371.
Neul J L1, Kaufmann W E, Glaze D G, Christodoulou J, Clarke A J, Bahi-Buisson N, Leonard H, Bailey M E, Schanen N C, Zappella M, Renieri A, Huppke P, Percy A K; RettSearch Consortium. Rett syndrome: revised diagnostic criteria and nomenclature. Ann Neurol. 2010 December; 68(6):944-50.
Breakthrough Therapy Designation Request Marinus Pharmaceuticals, Inc. Ganaxolone for the Treatment of CDKL5 23 Aug. 2017
Sartori S, Di Rosa G, Polli R, Bettella E, Tricomi G, Tortorella G, Murgia A. A novel CDKL5 mutation in a 47,XXY boy with the early-onset seizure variant of Rett syndrome. Am J Med Genet A. 2009; February; 149A(2):232-6.
Scala E, Ariani F, Mari F, Caselli R, Pescucci C, Longo I, Meloni I, Giachino D, Bruttini M, Hayek G, Zappella M, Renieri A. CDKL5/STK9 is mutated in Rett syndrome variant with infantile spasms. J Med Genet 2005; 42:103-107.
Tao J, Van Esch H, Hagedorn-Greiwe M, Hoffmann K, Moser B, Raynaud M, Sperner J, Fryns J P, Schwinger E, Gcz J, Ropers H H, Kalscheuer V M. Mutations in the X-linked cyclin-dependent kinase-like 5 (CDKL5/STK9) gene are associated with severe neurodevelopmental retardation. Am J Hum Genet. 2004; 75:1149-1154.
Tsai, J, Ligsay, A, Van Dijck, A, Kooy, F, Hessl, D, Bickel, E, Patroneva, A. A Randomized Double-blind, Placebo-controlled, Cross-over Trial of Ganaxolone in Children and Adolescents with Fragile X Syndrome (S46. 005). Neurology, 2017; 88 (16 Supplement), S46-005.
Van Esch H, Jansen A, Bauters M, Froyen G, Fryns J P. Encephalopathy and bilateral cataract in a boy with an interstitial deletion of Xp22 comprising the CDKL5 and NHS genes. Am J Med Genet A. 2007 Feb. 15; 143(4):364-9.
Weaving L S, Christodoulou J, Williamson S L, Friend K L, McKenzie O L, Archer H, Evans J, Clarke A, Pelka G J, Tam P P, Watson C, Lahooti H, Ellaway C J, Bennetts B, Leonard H, Gcz J. Mutations of CDKL5 cause a severe neurodevelopmental disorder with infantile spasms and mental retardation. Am J Hum Genet. 2004; 75:1079-1093.

1. A method of treating a mammal having an epileptic disorder, comprising
determining whether a mammal has a low level of an endogeneous neurosteroid, and
if the mammal has the low level of the endogenous neurosteroid, chronically administering a pharmaceutically acceptable pregnenolone neurosteroid to the mammal.

2. The method of claim 1, wherein the mammal is a human.

3. The method of claim 2, wherein the epileptic disorder is selected from the group consisting of CDKL5 deficiency disorder, PCDH19-related epilepsy, Lennox Gastaut Syndrome, Rett syndrome, and Fragile X Syndrome.

4. The method of claim 1, wherein the endogenous neurosteroid is allopregnanolone-sulfate, and the low level of the endogenous steroid is a level of 2500 pg mL⁻¹ or less.

5. The method of claim 1, wherein the pregnenolone neurosteroid is a compound of Formula IA: 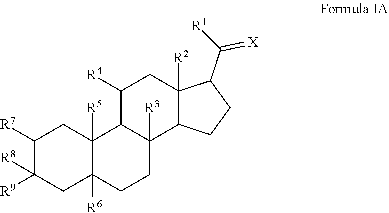
or a pharmaceutically acceptable salt thereof, wherein:
X is O, S, or NR¹⁰;
R¹ is hydrogen, hydroxyl, —CH₂A, optionally substituted alkyl, optionally substituted heteroalkyl, optionally substituted aryl, or optionally substituted arylalkyl;
A is hydroxyl, O, S, NR¹¹, optionally substituted nitrogen-containing five-membered heteroaryl, optionally substituted nitrogen-containing five-membered heteroaryl or optionally substituted nitrogen-containing bicyclic heteroaryl or bicyclic heterocyclyl,
R⁴ is hydrogen, hydroxyl, oxo, optionally substituted alkyl, or optionally substituted heteroalkyl,
R², R³, R⁵, R⁶, and R⁷ are each independently absent, hydrogen, hydroxyl, halogen, optionally substituted a C₁-C₆ alkyl, optionally substituted a C₁-C₆alkoxyl (e.g., methoxyl) or optionally substituted heteroalkyl;
R⁸ and R⁹ are each independently selected from a group consisting of hydrogen, a C₁-C₆ alkyl (e.g., methyl), a halogenated C₁-C₆ alkyl (e.g., trifluoromethyl) or C₁-C₆alkoxyl (e.g., methoxyl), or R⁸ and R⁹ form an oxo group;
R¹⁰ is hydrogen, hydroxyl, optionally substituted alkyl, optionally substituted heteroalkyl, optionally substituted aryl, or optionally substituted arylalkyl where each alkyl is a C₁-C₁₀alkyl, C₃-C₆cycloalkyl, (C₃-C₆cycloalkyl)C₁-C₄alkyl, and optionally contains a single bond replaced by a double or triple bond;
each heteroalkyl group is an alkyl group in which one or more methyl group is replaced by an independently chosen —O—, —S—, —N(R¹⁰)—, —S(═O)— or —S(═O)₂—, where R¹⁰ is hydrogen, alkyl, or alkyl in which one or more methylene group is replaced by —O—, —S—, —NH, or —N-alkyl;
R¹¹ is —H₂ or —HR¹²;
R¹² is C₁-C₆ alkyl or C₁-C₆ alkoxy.

6. The method of claim 2, wherein the pregnenolone neurosteroid is selected from the group consisting of allopregnanolone, pregnenolone, 5-alphaDHP (5-alphadihydroprogesterone), pregnanolone, dehydroepiandrosterone (DHEA), ganaxolone, 3α-Hydroxy-3β-methyl-21-(4-cyano-1H-pyrazol-1′-yl)-19-nor-5β-pregnan-20-one, pharmaceutically acceptable salts of any of the foregoing, and combinations of any of the foregoing.

7. The method of claim 6, wherein the pregnenolone neurosteroid is ganaxolone.

8. The method of claim 7, wherein ganaxolone is administered orally.

9. The method of claim 7, wherein ganaxolone is administered as an oral suspension up to a total of 63 mg/kg/day.

10. A method of treating an epileptic encephalopathy, comprising
identifying a human patient suffering from the epileptic encephalopathy,
determining if the human patient has a low level of an endogenous neurosteroid, and
if the human patient has a low level of an endogenous neurosteroid, administering the human patient a dosage regimen of a pharmaceutically acceptable pregnenolone neurosteroid in an amount effective to reduce the frequency of seizures in the human patient.

11. The method of claim 10, wherein the epileptic encephalopathy is CDKL5 deficiency disorder.

12. The method of claim 1, wherein the human patient has a CDKL5 genetic mutation.

13. The method of claim 10, wherein the epileptic encephalopathy is selected from the group consisting of CDKL5 deficiency disorder, PCDH19-related epilepsy, Lennox-Gastaut Syndrome, Ohtahara syndrome, early myoclonic epileptic encephalopathy, West syndrome, Dravet syndrome, Angelman Syndrome, and other diseases, e.g., X-linked myoclonic seizures, spasticity and intellectual disability syndrome, idiopathic infantile epileptic-dyskinetic encephalopathy, epilepsy and mental retardation limited to females, and severe infantile multifocal epilepsy.

14. The method of claim 10, wherein the pregnenolone neurosteroid is a compound of Formula IA: 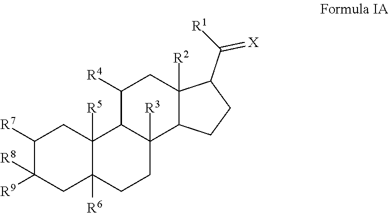
or a pharmaceutically acceptable salt thereof, wherein:
X is O, S, or NR¹⁰;
R¹ is hydrogen, hydroxyl, —CH₂A, optionally substituted alkyl, optionally substituted heteroalkyl, optionally substituted aryl, or optionally substituted arylalkyl;
A is hydroxyl, O, S, NR¹¹, optionally substituted nitrogen-containing five-membered heteroaryl, or optionally substituted nitrogen-containing bicyclic heteroaryl or bicyclic heterocyclyl,
R⁴ is hydrogen, hydroxyl, oxo, optionally substituted alkyl, or optionally substituted heteroalkyl,
R², R³, R⁵, R⁶, and R⁷ are each independently absent, hydrogen, hydroxyl, halogen, optionally substituted a C₁-C₆ alkyl, optionally substituted a C₁-C₆alkoxyl (e.g., methoxyl) or optionally substituted heteroalkyl;
R⁸ and R⁹ are each independently selected from a group consisting of hydrogen, a C₁-C₆ alkyl (e.g., methyl), a halogenated C₁-C₆ alkyl (e.g., trifluoromethyl) or C₁-C₆alkoxyl (e.g., methoxyl), or R⁸ and R⁹ form an oxo group;
R¹⁰ is hydrogen, hydroxyl, optionally substituted alkyl, optionally substituted heteroalkyl, optionally substituted aryl, or optionally substituted arylalkyl where each alkyl is a C₁-C₁₀alkyl, C₃-C₆cycloalkyl, (C₃-C₆cycloalkyl)C₁-C₄alkyl, and optionally contains a single bond replaced by a double or triple bond;
each heteroalkyl group is an alkyl group in which one or more methyl group is replaced by an independently chosen —O—, —S—, —N(R¹⁰)—, —S(═O)— or —S(═O)₂—, where R¹⁰ is hydrogen, alkyl, or alkyl in which one or more methylene group is replaced by —O—, —S—, —NH, or —N-alkyl;
R¹¹ is —H₂ or —HR¹²;
R¹² is C₁-C₆ alkyl or C₁-C₆ alkoxy.

15. The method of claim 10, wherein the pregnenolone neurosteroid is selected from the group consisting of pregnenolone, 5-alphaDHP (5-alphadihydroprogesterone), pregnanolone, dehydroepiandrosterone (DHEA), ganaxolone, 3α-Hydroxy-3β-methyl-21-(4-cyano-1H-pyrazol-1′-yl)-19-nor-5β-pregnan-20-one, pharmaceutically acceptable salts of any of the foregoing, and combinations of any of the foregoing.

16. The method of claim 15, wherein the pregnenolone neurosteroid is ganaxolone.

17. The method of claim 10, further comprising:
establishing a baseline seizure frequency,
initially administering a dose of ganaxolone to the patient in an amount from about 0.5 mg/kg/day to about 15 mg/kg/day; and
progressively increasing the dose of ganaxolone over the course of 4 weeks to an amount from about 18 mg/kg/day to about 60 mg/kg/day, wherein the total dose of ganaxolone is up to about 1800 mg/day.

18. The method of claim 10, wherein the endogenous neurosteroid is allopregnanolone-sulfate, and the low level of the endogenous neurosteroid is a level of 2500 pg mL⁻¹ or less.

19-20. (canceled)

21. A method for reducing seizures associated with CDKL5 deficiency disorder, comprising administering to a subject in need thereof ganaxolone in an amount of about one week followed by increasing the amount of ganaxolone about once per week for a duration of about three subsequent weeks to an amount of about 63 mg/kg/day, wherein ganaxolone is administered orally three times per day.
